(19) United States
(12) Patent Application Publication
(10) Pub. No.: US 2017/0313990 A1
(43) Pub. Date: Nov. 2, 2017

(54) A PROCESS FOR THE PRODUCTION OF ADENOVIRUS

(71) Applicant: PSIOXUS THERAPEUTICS LIMITED, Oxfordshire (GB)

(72) Inventors: Brian Robert CHAMPION, Abingdon, Oxfordshire (GB); Jeetendra BHATIA, Abingdon, Oxfordshire (GB); Ashvin PATEL, Abingdon, Oxfordshire (GB)

(73) Assignee: PSIOXUS THERAPEUTICS LIMITED, Oxfordshire (GB)

(21) Appl. No.: 15/506,194

(22) PCT Filed: Aug. 27, 2015
(86) PCT No.: PCT/EP2015/069706

§ 371 Date: Feb. 23, 2017

(30) Foreign Application Priority Data
Aug. 27, 2014 (GB) 1415154.2
Sep. 3, 2014 (GB) 1415581.6

(51) Int. Cl. C12N 7/00

(52) U.S. Cl. CPC C12N 7/00; C12N 2710/10021; C12N 2710/10051

(57) ABSTRACT

A Process for the Production of Adenovirus The present disclosure relates to a continuous process for the manufacture of an adenovirus wherein the process comprises the steps: A) continuously culturing, in a vessel, mammalian cells infected with the adenovirus in the presence of media suitable for supporting the cells such that the virus replicates, wherein the cells are capable of supporting viral replication, and B) isolating from the media the virus produced from step a) wherein the isolation of virus is not subsequent to a cell lysis step, wherein viable cells for virus infection and production are maintained in the vessel at a level suitable for replicating the virus for the period of continuous manufacture, wherein the process comprises at least one media change or addition and at least one cell change or addition. The disclosure also extends to viruses populations obtained or obtainable from the method.

The present disclosure relates to a method for the manufacture of certain adenoviruses, for example chimeric adenoviruses and/or replication competent adenoviruses, and/or group B viruses and the viral product obtained therefrom.

The present specification claims priority from GB1415154.2 filed 27 Aug. 2014 and GB1415581.6 filed 3 Sep. 2014 both of which are incorporated herein by reference.

### BACKGROUND

At the present time the pharmaceutical field is on the edge of realising the potential of viruses as therapeutics for human use. To date a virus derived from ONXY-15 (ONYX Pharmaceuticals and acquired by Shanghai Sunway Biotech) is approved for use in head and neck cancer in a limited number of countries. However, there are a number of viruses currently in the clinic, which should hopefully result in some of these being registered for use in humans.

A number of virus therapies are based on adenoviruses, for example EnAd (ColoAd1) is a chimeric oncolytic adenovirus (WO2005/118825) currently in clinical trials for the treatment of colorectal cancer.

These adenoviral based therapeutic agents need to be manufactured in quantities suitable for supporting both the clinical trials and demand after registration and under conditions that adhere to good manufacturing practice (GMP).

As part of the manufacturing process, the viruses are propagated in mammalian cells in vitro, for example in a cell suspension culture. The virus is recovered from these cells by cell lysis and subsequent purification. FIG. 1 is an extract from Kamen & Henry 2004 (J Gene Med. 6: pages 184-192) showing a schematic diagram of the processes involved manufacture of the GMP grade adenovirus. Notably, after viral replication, the cells are lysed.

Contaminating DNA and host cell protein (HCP) from the cells after lysis can be a significant problem and must be removed as far as possible from the final therapeutic adenoviral product. This is described in detail in the application WO2011/045381, which describes lysing the cells, fragmenting or precipitating the DNA within the cell suspension and clarifying the same, employing tangential flow. DNA digestion with DNAse is also shown as the third step in FIG. 1.

Developing a successful recombinant adenovirus process requires a detailed understanding of basic host cell line physiology and metabolism; the recombinant virus, and the interaction between the cell line and the virus. Essentially the process requires adaptation depending on the particular type of virus or viral vector.

In addition the cost of manufacturing recombinant virus suitable for clinical use is relatively high. Improved processes that increase the efficiency of manufacture, for example increase the yield of virus are required to ensure clinical demand can be met for recombinant viral products that gain regulatory approval and to reduce manufacturing costs.

Surprisingly the present inventors have established that certain adenoviruses, for example replication competent adenoviruses and chimeric oncolytic adenoviruses, and group B adenoviruses can be prepared by a continuous process that isolates the virus from the cell media and that avoids the necessity to lyse the cells and thus may significantly reduce the starting levels of DNA and HCP contamination in the viral product.

In addition the present inventors have established parameters that give them control over where the virus, for example group B virus product, is located in the culture at a given time point i.e. in the supernatant or associated with the cell pellet. This allows the processes to be adapted as required.

### SUMMARY OF THE INVENTION

Thus the present disclosure provides a continuous process for the manufacture of:

an adenovirus (for example a group B adenovirus) wherein the process comprises the steps:
a. continuously culturing, in a vessel, mammalian cells infected with the adenovirus in the presence of media suitable for supporting the cells such that the virus replicates, wherein the cells are capable of supporting viral replication, and
b. isolating from the media the virus produced from step a) wherein the isolation of virus is not subsequent to a cell lysis step,
   wherein viable cells for virus infection and production are maintained in the vessel at a level suitable for replicating the virus for the period of continuous manufacture.

Thus the present disclosure provides a continuous process for the manufacture of a virus selected from the group consisting of a replication competent adenovirus; a group B virus, an adenovirus which does not encode or does not express an adenovirus death protein, a replication capable or deficient chimeric oncolytic adenovirus having a genome comprising an E2B region, wherein said E2B region comprises a nucleic acid sequence derived from a first adenoviral serotype and a nucleic acid sequence derived from a second distinct adenoviral serotype; wherein said first and second serotypes are each selected from the adenoviral subgroups B, C, D, E, or F, and combinations of two or more of the same, wherein the process comprises the steps:
a. continuously culturing, in a vessel, mammalian cells infected with the adenovirus in the presence of media suitable for supporting the cells such that the virus replicates, wherein the cells are capable of supporting viral replication, and
b. isolating from the media the virus produced from step a) wherein the isolation of virus is not subsequent to a cell lysis step,
   wherein viable cells for virus infection and production are maintained in the vessel at a level suitable for replicating the virus for the period of continuous manufacture.
   Thus in one embodiment the present disclosure provides a continuous process for the manufacture of:
   a replication competent adenovirus; or
   a replication capable or deficient chimeric oncolytic adenovirus having a genome comprising an E2B region, wherein said E2B region comprises a nucleic acid sequence derived from a first adenoviral serotype and a nucleic acid sequence derived from a second distinct adenoviral serotype; wherein said first and second serotypes are each selected from the adenoviral subgroups B, C, D, E, or F, wherein the process comprises the steps:
a. continuously culturing, in a vessel, mammalian cells infected with the adenovirus in the presence of media suitable for supporting the cells such that the virus replicates, wherein the cells are capable of supporting viral replication, and
b. isolating from the media the virus produced from step a) wherein the isolation of virus is not subsequent to a cell lysis step,
   wherein viable cells for virus infection and production are maintained in the vessel at a level suitable for replicating the virus for the period of continuous manufacture.
In one independent aspect there is provided a continuous process for the manufacture of a type B adenovirus wherein the process comprises the steps:
a. continuously culturing, in a vessel, mammalian cells infected with the adenovirus in the presence of media suitable for supporting the cells such that the virus replicates, wherein the cells are capable of supporting viral replication, and
b. isolating from the media the virus produced from step a) wherein the isolation of virus is not subsequent to a cell lysis step,
   wherein viable cells for virus infection and production are maintained in the vessel at a level suitable for replicating the virus for the period of continuous manufacture.

In one embodiment the type B adenovirus is a chimeric oncolytic adenovirus having a genome comprising an E2B region, wherein said E2B region comprises a nucleic acid sequence derived from a first adenoviral serotype and a nucleic acid sequence derived from a second distinct adenoviral serotype; wherein said first and second serotypes are each selected from the adenoviral subgroups B, C, D, E, or F.

In one embodiment the oncolytic virus has a fibre, hexon and penton proteins from the same serotype, for example Ad11, in particular Ad11p, for example found at positions 30812-31789, 18254-21100 and 13682-15367 of the genomic sequence of the latter wherein the nucleotide positions are relative to genbank ID 217307399 (accession number: GC689208).

In one embodiment the adenovirus is enadenotucirev (also known as EnAd and formerly as EnAd). Enadenotucirev as employed herein refers the chimeric adenovirus of SEQ ID NO: 12. It is a replication competent oncolytic chimeric adenovirus which has enhanced therapeutic properties compared to wild type adenoviruses (see WO2005/118825). EnAd has a chimeric E2B region, which features DNA from Ad11p and Ad3, and deletions in E3/E4. The structural changes in enadenotucirev result in a genome that is approximately 3.5 kb smaller than Ad11p thereby providing additional “space” for the insertion of transgenes.

OvAd1 and OvAd2 are also chimeric adenoviruses similar to enadenotucirev, which also have additional “space” in the genome (see WO2008/080003). Thus in one embodiment the adenovirus is OvAd1 or OvAd2.

In one embodiment the group B adenovirus comprises a genome comprising formula (I):

5′ITR-B₁-B_{A}-B₂-B_{X}-B_{B}-B_{Y}-B₃-3′ITR (I)

wherein:
B₁ is a bond or comprises: E1A, E1B or E1A-E1B;

### B_{A} is E2B-L1-L2-L3-E2A-L4;

B₂ is a bond or comprises E3;
B_{X} is a bond or a DNA sequence comprising: a restriction site, one or more transgenes or both;
B_{B} comprises L5;
B_{Y} is a bond or a DNA sequence comprising: a restriction site, one or more transgenes or both;
B₃ is a bond or comprises E4;
wherein at least one of B_{X} and B_{Y} is not a bond, for example at least one of B_{X} and B_{Y} comprises a transgene, a restriction site or both, such as a transgene.
Thus in one embodiment there is provided a continuous process according to the present disclosure, wherein the type B adenovirus is replication competent.

A replication competent group B adenovirus comprising a sequence of formula (I):

5′ITR-B₁-B_{A}-B₂-B_{X}-B_{B}-B_{Y}-B₃-3′ITR

wherein:
B₁ is bond or comprises: E1A, E1B or E1A-E1B;
B_{A} comprises-E2B-L1-L2-L3-E2A-L4;
B₂ is a bond or comprises: E3;
B_{X} is a bond or a DNA sequence comprising: a restriction site, one or more transgenes or both;
B_{B} comprises L5;
B_{Y} comprises a transgene cassette comprising a transgene and a splice acceptor sequence; and
B₃ is a bond or comprises: E4,
   wherein the transgene cassette is under the control of an endogenous promoter selected from the group consisting of E4 and major late promoter and wherein the transgene cassette does not comprise a non-biasedly inserting transposon.

Viruses of formula (I) are disclosed in WO2015/059303 incorporated herein by reference.

In one embodiment the adenovirus is a sequence disclosed herein in the sequence listing.

In one embodiment the virus employed in the process of the present disclosure contains less than a full length adenovirus genome, for example contains 50%, 60%, 70%, 80% or more of an adenovirus genome or a sequence that hybridises to 50%, 60%, 70%, 80% or more of an adenovirus genome under stringent conditions.

In one embodiment the virus of the present disclosure has part or all of the E3 region deleted. Whilst not wishing to be bound by theory it is thought by the inventors that partial or complete deletion of this region may speed up the rate of viral replication, which in some instances may provide beneficial properties in vivo.

In one embodiment the virus employed in the present disclosure has part or all of the E4 region deleted. Whilst not wishing to be bound by theory it is thought by the inventors that the partial deletion of the E4 region may speed up the rate of viral replication, which in some instances may provide beneficial properties in vivo.

In one embodiment the virus employed in the present disclosure is partially deleted in the E4 region such that the virus retains its replication competency.

In one embodiment the virus employed in the present disclosure has part or all of the E3 region deleted and part or all of the E4 region deleted, as appropriate.

In one embodiment the adenovirus has a hexon and fibre from a group B adenovirus, for example Ad11 or EnAd.

In one embodiment there is provided is a continuous process for the manufacture of an adenovirus having a fibre and hexon of subgroup B (such as Ad11, in particular Ad11p also known as the Slobitski strain) for example wherein part or all of the E3 and/or part of all E4 region is deleted and said process comprises the steps:
a. continuously-culturing in a vessel mammalian cells infected with the adenovirus in the presence of media suitable for supporting the cells such that the virus replicates, wherein the cells are capable of supporting viral replication, and
b. isolating from the media the virus from step a) wherein the isolation of virus is not subsequent to a cell lysis step,
   wherein viable cells for virus infection and production are maintained in the culture at a level suitable for replicating the virus for the period of continuous manufacture.

In one embodiment a virus employed the present disclosure may comprise a transgene.

In one embodiment the adenovirus of the present disclosure, such as the type B adenovirus is a replication capable or deficient.

In one embodiment the adenovirus is replication competent or replication deficient, for example replication competent. Replication deficient adenoviruses are also referred to herein as viral vectors.

In one embodiment the chimeric virus is replication competent or replication deficient, for example replication competent.

In one embodiment the virus employed in the present disclosure does not express a functional adenovirus death protein or a functional fragment thereof, in particular does not express an adenovirus death protein or a fragment thereof.

In one embodiment the virus of the present disclosure does not comprise a DNA sequence encoding a functional adenovirus death protein or a functional fragment thereof, in particular does not comprise a sequence encoding an adenovirus death protein or fragment thereof.

In one embodiment the adenovirus employed in the present disclosure is not one which infects cells via the coxsackievirus and adenovirus receptor (CAR).

In one embodiment the adenovirus employed in the present disclosure is one which infects cells via CD46, for example group B adenoviruses.

In one embodiment the virus, for example replication competent virus is not a group C virus.

In one embodiment the virus, for example replication competent virus is not Ad5.

In one embodiment the continuous manufacture period is at least two virus cycles, for example 70 to 300 hours.

In one embodiment the process comprises at least two harvesting steps.

In one embodiment the process comprises continuous harvesting of virus, which is initiated at least 24 hours, such as 30 hours or 35 hours post infection and, for example continued until the end of the process.

In one embodiment, for example at the end of the process there is a single cell lysis step, in particular to recover virus retained in the cell.

In one embodiment there process comprises combining one or more fractions virus harvested.

In one embodiment the process comprises a step of adding fresh cells to the culture on one or more occasions, for example one, two, three, four or five additions, for example independently selected at one or more time points, such as 24, 30, 35, 40, 45, 48, 50, 55, 60, 65, 70, 72, 75, 80, 85, 90, 95 or 96 hours.

In one embodiment the process comprises at least one step where fresh media is added. In one embodiment the process comprises at least one media change or addition, for example at any time point between 12 and 96 hours after infection, such as 24, 30, 35, 40, 45, 48, 50, 55, 60, 65, 70, 72, 75, 80, 85, 90, 95 or 96 hours.

In one embodiment the process comprises a step of:

adding fresh cells to the culture on one or more occasions, for example one, two, three, four or five additions, for example independently selected at one or more time points, such as 24, 30, 35, 40, 45, 48, 50, 55, 60, 65, 70, 72, 75, 80, 85, 90, 95 or 96 hours, and
adding fresh media or changing the media example at any time point between 12 and 96 hours after infection, such as 24, 30, 35, 40, 45, 48, 50, 55, 60, 65, 70, 72, 75, 80, 85, 90, 95 or 96 hours.

In one embodiment the cells are infected with a starting concentration of virus of 1-9×10⁴ vp/ml or greater, such as 1-9×10⁵, 1-9×10⁶, 1-9×10⁷, 1-9×10⁸, 1-9×10⁹ vp/ml, in particular 1 to 5×10⁷ vp/ml, including about 1×10⁶, 4 to 5×10⁶, in particular as 1×10⁶ vp/ml.

In one embodiment the multiplicity of infection (MOI) is in the range 2 to 75, for example 5, 6, 7, 8, 9, 10, 11, 12, 12.5, 13, 14, 15, 16, 17, 18, 19, 20, 21, 22, 23, 24, 25, 26, 27, 28, 29, 30, 31, 31.25, 32, 33, 35, 36, 37, 38, 39, 40, 41, 42, 43, 44, 45, 46, 47, 48, 49, 50, 51, 52, 53, 54, 55 etc, such as 12.5, 31.25 or 50. In one embodiment the multiplicity of infection is in the range 10 to 15, such as 12.5 vp/cell.

In one embodiment the MOI is in the range 2 to 50, for example 5 to 20, such as 6, 7, 8, 9, 10, 11, 12, 13, 14 or 15, such as 12.5 vp/cell

In one embodiment the seed density is 1.9×10⁶ or 4×10⁶ and the multiplicity of infection is 50.

In one embodiment the seed density in the range 1-1.9×10⁶ such as 1×10⁶ vp/ml and the multiplicity of infection in the range 10-15, such as 12.5.

In one embodiment of the present disclosure the culture employed is perfusion culture.

In one embodiment of the present disclosure the cells employed are adherent.

In one embodiment of the present disclosure the cells employed are suitable for suspension culture.

In one embodiment the culture employed in the process of the present disclosure is a suspension culture, adherent culture, perfusion culture or combinations thereof, such as suspension culture.

The continuous manufacturing processes according to the present disclosure is advantageous in that it has one or more of the following benefits: increases the efficiency of manufacturing by allowing adequate quantities of virus to be prepared for clinical use, reduces time spent in manufacturing campaigns, which in turn reduces cost of goods, and also reduces the complexity of manufacturing in that it minimises the need to prepare multiple master viral seed stocks, which may also reduce costs.

Other advantages of the process described herein include the ability for the scale of the process to be reduced due to, for example due to the increased yields.

Furthermore the present inventors have established parameters that allow the cells to produce high levels of virus per cell. In this independent aspect the culture is, for example, characterised in that it has a low multiplicity of infection in combination with a low starting seed cell density.

Thus in one independent aspect there is provided a method of infecting cells suitable for replicating adenovirus wherein the starting seed density is 2×10⁶ vp/ml or less, for example 1.5×10⁶ vp/ml or less such as 1×10⁶ vp/ml and a multiplicity of infection of 15 or less, such as 14, 13, 12.5, 12, 11 or 10, such as 12.5.

Thus in one independent aspect there is provided a method of infecting cells suitable for replicating adenovirus wherein the starting seed density is 1.9×10⁶ and a multiplicity of infection of 50 ppc.

Yields as high as 200,000 virus particles per cell at certain time points, such as about 72 hours or more may be achieved employing conditions described herein. In one embodiment the virus produced per cell at over 100,000, for example after 48, 50, 55, 60, 65 or 70 hours.

In this embodiment where the culture employs a low seed density and a low multiplicity of infection the virus can readily be harvested.

In addition there present inventors have established that by altering the parameters of the process control can be provided over where the virus is located, for example in the cell, in the supernatant or a combination thereof. This forms a further aspect of the present disclosure.

In particular low multiplicity of infection with low seed density results in the virus product located predominantly in the cell, however towards the end of the process the virus is also found in the supernatant. It also gave a very high yield per cell, especially when the media was changed.

High multiplicity of infection and high seed density also results in the majority of virus particles in the supernatant towards the end of the process.

High multiplicity of infection in combination with a low seed density provides most the virus product in the supernatant towards the end of the process.

In contrast low multiplicity of infection with high seed density gave virus in the cell, in particular when the media was not changed.

Moderate multiplicities of infection in combination with moderate seed densities provide a virus product in the supernatant and the cell, in particular when there was no media change.

Furthermore high multiplicity of infection and high seed density appears to provide virus product primarily in the supernatant, with or without a change of media.

Thus surprisingly by controlling the parameters of the process the location of the virus product can be controlled. In one embodiment there is provided a method for controlling the partitioning of a recombinant virus (in particular an adenovirus described herein) between the supernatant and a host cell, which method comprises: a) providing a host cell culture (in particular a mammalian cell, such as one described herein, in particular as a suspension culture) b) selecting a seed density, multiplicity of infection and duration of infection (duration of culture) to provide virus product in the desired location selected from the supernatant and the host cell c) determining the yield of recombinant virus in the culture supernatant and the host cell at a relevant time point and d) comparing the yield in the supernatant and the cell determined in step (c) electing to keep the seed density and multiplicity of infection selected in step b) or changing the seed density, multiplicity of infection or both to alter the partitioning of the recombinant virus between the supernatant and the cell to suit to the primary recovery of the recombinant virus.

In one embodiment the process of controlling the parameters according to the present disclosure comprises steps performed in parallel for different conditions i.e. a process where the seed density, multiplicity of infection or both are changed from the parameters employed in a first process, thereby allowing comparison of two different processes.

In one embodiment 80% or more of the recombinant virus is in the supernatant, for example 85, 86, 87, 88, 89, 90, 91, 92, 93, 94, 95, 96, 97, 98, 99 or 100% is located in the supernatant.

In one embodiment 80% or more of the recombinant virus is in the cell for example 85, 86, 87, 88, 89, 90, 91, 92, 93, 94, 95, 96, 97, 98, 99 or 100% is located in the cell, for example when a low multiplicity of infection is employed and a low seed density is employed as defined herein, over a duration of infection of 48-65 hours. A media change or addition may also help to maximise the yield.

Thus in one independent aspect there is provided a process for the manufacture of an adenovirus (for example a virus described herein such as a group B adenovirus) wherein the process comprises the steps:

a. culturing, in a vessel, mammalian cells infected with the adenovirus in the presence of media suitable for supporting the cells such that the virus replicates, wherein the cells are capable of supporting viral replication, wherein the starting seed density of the virus is in the range 1 to 2×10⁶ vp/ml (such as 1×10⁶ vp/ml) and the multiplicity of infection is in the range 5 to 20, such as 10 to 15, in particular 12.5; and

b. performing a lysis step in the period 24 to 75 hours post virus infection to harvest the virus from the cells, for example where the lysis step is performed at 65 to 70 hours post infection, such as 66, 67, 68 or 69 hours post infection.

In one embodiment the process is a GMP manufacturing process.

### DETAILED DESCRIPTION OF THE DISCLOSURE

Virus of the present disclosure is generally employed herein to refer generically to replication capable, replication competent or replication deficient adenovirus including a chimeric oncolytic adenovirus unless the context indicates otherwise.

Replication capable as employed herein refers to a replication competent virus or virus which can selectively replicate in a cell. Viruses which selectively replicate in cancer cells are those which require a gene or protein which is upregulated in a cancer cell to replicate, such as a p53 gene.

Replication competent virus as employed herein refers to a virus that is capable of replication without the assistance of a complementary cell line encoding an essential viral protein, such as that encoded by the E1 region (also referred to as a packaging cell line) and virus capable of replicating without the assistance of a helper virus.

In one embodiment the virus is replication competent.

A replication deficient virus is a vector and requires the use of a packaging cell line or helper virus to be able to replicate.

Adenovirus as employed will generally refer to a replication competent adenovirus or replication deficient, for example a group B virus, in particular Ad11, such as Ad11p, unless the context indicates otherwise. In some instances it may be employed to refer to refer only to replication competent viruses and this will be clear from the context.

In one embodiment the adenovirus is replication competent.

Adenovirus vector will generally refer to a replication deficient adenovirus.

Subgroup B (group B or type B) as employed herein refers to viruses with at least the fibre and hexon from a group B adenovirus, for example the whole capsid from a group B virus, such as substantially the whole genome from a group B virus. In one embodiment a group B virus does not encode an adenovirus death protein, for example an ADP protein of 11.64 KDaltons, such as the protein with Uniprot number P24935.

All human adenovirus genomes examined to date have the same general organisation i.e., the genes encoding specific functions are located at the same position in the viral genome (referred to herein as structural elements). Each end of the viral genome has a short sequence known as the inverted terminal repeat (or ITR), which is required for viral replication. The viral genome contains five early transcription units (E1A, E1B, E2, E3, and E4), three delayed early units (IX, IVa2 and E2 late) and one late unit (major late) that is processed to generate five families of late mRNAs (L1-L5). Proteins encoded by the early genes are primarily involved in replication and modulation of the host cell response to infection, whereas the late genes encode viral structural proteins. Early genes are prefixed by the letter E and the late genes are prefixed by the letter L.

The genome of adenoviruses is tightly packed, that is, there is little non-coding sequence, and therefore it can be difficult to find a suitable location to insert transgenes. The present inventors have identified two DNA regions where transgenes are tolerated, in particular the sites identified are suitable for accommodating complicated transgenes, such as those encoding antibodies. That is, the transgene is expressed without adversely affecting the virus' viability, native properties such as oncolytic properties or replication, that is position B_{X} and/or position B_{Y} in viruses of formula (I).

In one embodiment the adenovirus is partially or completely deleted in the E3 region.

Part of the E3 region is deleted as employed herein means that at least part, for example in the range 1 to 99% of the E3 region is deleted, such as 2, 3, 4, 5, 6, 7, 8, 9, 10, 15, 20, 25, 30, 35, 40, 45, 50, 55, 60, 65, 70, 75, 80, 85, 90, 91, 92, 93, 94 95, 96, 97 or 98% deleted.

In one embodiment the adenovirus is partially deleted in the E4 region. Viruses can be maintained as replication competent when only part of the E4 region is deleted.

Part of the E4 region is deleted as employed herein means that at least part, for example in the range 1 to 99% of the E4 region is deleted, such as 2, 3, 4, 5, 6, 7, 8, 9, 10, 15, 20, 25, 30, 35, 40, 45, 50, 55, 60, 65, 70, 75, 80, 85, 90, 91, 92, 93, 94 95, 96, 97 or 98% deleted. In adenoviruses of the present disclosure sufficient E4 should be retained to allow replication.

Chimera (or chimeric virus) as employed herein will generally refer to an adenovirus comprising genomic DNA from at least two different serotypes, for example serotypes independently selected from groups B, C, D, E and F, such as replication capable, replication competent or replication deficient chimeric oncolytic virus. In one embodiment the chimeric oncolytic adenovirus is replication competent, for example EnAd, OvAd1 or OvAd2.

In one embodiment the chimeric virus is EnAd (SEQ ID NO: 12) or a derivative thereof, for example a derivate adapted to incorporate a transgene or transgenes, examples of which are discussed below.

In one embodiment the chimera is partially or completely deleted in the E3 region.

Part of the E3 region is deleted as employed herein means that at least part, for example in the range 1 to 99% of the E3 region is deleted, such as 2, 3, 4, 5, 6, 7, 8, 9, 10, 15, 20, 25, 30, 35, 40, 45, 50, 55, 60, 65, 70, 75, 80, 85, 90, 91, 92, 93, 94 95, 96, 97 or 98% deleted.

In one embodiment the chimera is partially or completely deleted in the E4 region.

Part of the E4 region is deleted as employed herein means that at least part, for example in the range 1 to 99% of the E4 region is deleted, such as 2, 3, 4, 5, 6, 7, 8, 9, 10, 15, 20, 25, 30, 35, 40, 45, 50, 55, 60, 65, 70, 75, 80, 85, 90, 91, 92, 93, 94 95, 96, 97 or 98% deleted. In adenoviruses of the present disclosure sufficient E4 should be retained to allow replication.

Transgene as employed herein refers to a gene that has been inserted into the genome sequence, which is a gene that is unnatural to the virus (exogenous) or not normally found in that particular location in the virus. Examples of transgenes are given below. Transgene as employed herein also includes a functional fragment of the gene that is a portion of the gene which when inserted is suitable to perform the function or most of the function of the full-length gene.

Transgene and coding sequence are used interchangeably herein in the context of inserts into the viral genome, unless the context indicates otherwise. Coding sequence as employed herein means, for example a DNA sequence encoding a functional RNA, peptide, polypeptide or protein. Typically the coding sequence is cDNA for the transgene that encodes the functional RNA, peptide, polypeptide or protein of interest. Functional RNA, peptides, polypeptide and proteins of interest are described below.

Clearly the virus genome contains coding sequences of DNA. Endogenous (naturally occurring genes) in the genomic sequence of the virus are not considered a transgene, within the context of the present specification unless then have been modified by recombinant techniques such as that they are in a non-natural location or in a non-natural environment or they have a non-natural function.

In one embodiment transgene as employed herein refers to a segment of DNA containing a gene or cDNA sequence that has been isolated from one organism and is introduced into a different organism i.e. the virus of the present disclosure. In one embodiment this non-native segment of DNA may retain the ability to produce functional RNA, peptide, polypeptide or protein.

Thus in one embodiment the transgene inserted encodes a human or humanised protein, polypeptide or peptide.

In one embodiment the transgene inserted encodes a non-human protein, polypeptide or peptide (such as a non-human mammalian protein, polypeptide or peptide) or RNA molecule, for example from a mouse, rat, rabbit, camel, llama or similar. Advantageously, the viruses of the present disclosure allow the transgenes to be transported inside the cancerous cell. Thus, responses generated by the human patient to a non-human sequence (such as a protein) can be minimised by this intra-cellular deliver

A DNA sequence may comprise more than one transgene, for example, 1, 2, 3 or 4 transgenes, such as 1 or 2.

A transgene cassette may comprise more than one transgene, for example, 1, 2, 3 or 4 transgenes, such as 1 or 2.

Transgene cassette as employed herein refers to a DNA sequence encoding one or more transgenes in the form of one or more coding sequences and one or more regulatory elements.

A transgene cassette may encode one or more monocistronic and/or polycistronic mRNA sequences.

In one embodiment the transgene or transgene cassette encodes a monocistronic or polycistronic mRNA, and for example the cassette is suitable for insertion into the adenovirus genome at a location under the control of an endogenous promoter or exogenous promoter or a combination thereof.

Monocistronic mRNA as employed herein refers to an mRNA molecule encoding a single functional RNA, peptide, polypeptide or protein.

In one embodiment the transgene cassette encodes monocistronic mRNA.

In one embodiment the transgene cassette in the context of a cassette encoding monocistronic mRNA means a segment of DNA optionally containing an exogenous promoter (which is a regulatory sequence that will determine where and when the transgene is active) or a splice site (which is a regulatory sequence determining when a mRNA molecule will be cleaved by the spliceosome) a coding sequence (i.e. the transgene), usually derived from the cDNA for the protein of interest, optionally containing a polyA signal sequence and a terminator sequence.

In one embodiment the transgene cassette may encode one or more polycistronic mRNA sequences.

Polycistronic mRNA as employed herein refers to an mRNA molecule encoding two or more functional RNA, peptides or proteins or a combination thereof. In one embodiment the transgene cassette encodes a polycistronic mRNA.

In one embodiment transgene cassette in the context of a cassette encoding polycistronic mRNA includes a segment of DNA optionally containing an exogenous promoter (which is a regulatory sequence that will determine where and when the transgene is active) or a splice site (which is a regulatory sequence determining when a mRNA molecule will be cleaved by the spliceosome) two or more coding sequences (i.e. the transgenes), usually derived from the cDNA for the protein or peptide of interest, for example wherein each coding sequence is separated by either an IRES or a 2A peptide. Following the last coding sequence to be transcribed, the cassette may optionally contain a polyA sequence and a terminator sequence.

In one embodiment the transgene cassette encodes a monocistronic mRNA followed by a polycistronic mRNA. In another embodiment the transgene cassette a polycistronic mRNA followed by a monocistronic mRNA.

In one embodiment B_{X} comprises a restriction site, for example 1, 2, 3 or 4 restriction sites, such as 1 or 2. In one embodiment B_{X} comprises at least one transgene, for example 1 or 2 transgenes. In one embodiment B_{X} comprises at least one transgene, for example 1 or 2 transgenes and one or more restriction sites, for example 2 or 3 restriction sites, in particular where the restrict sites sandwich a gene or the DNA sequence comprising the genes to allow it/them to be specifically excised from the genome and/or replaced. Alternatively, the restriction sites may sandwich each gene, for example when there are two transgenes three different restriction sites are required to ensure that the genes can be selectively excised and/or replaced. In one embodiment one or more, for example all the transgenes are in the form a transgene cassette. In one embodiment B_{X} comprises SEQ ID NO: 10. In one embodiment SEQ ID NO: 10 is interrupted, for example by a transgene. In embodiment SEQ ID NO: 10 is uninterrupted. In one embodiment B_{X} does not comprise a restriction site. In one embodiment B_{X} is a bond. In one embodiment B_{X} comprises or consists of one or more transgenes.

In one embodiment B_{Y} comprises a restriction site, for example 1, 2, 3 or 4 restriction sites, such as 1 or 2. In one embodiment B_{Y} comprises at least one transgene, for example 1 or 2 transgenes. In one embodiment B_{Y} comprises at least one transgene, for example 1 or 2 transgenes and one or more restriction sites, for example 2 or 3 restriction sites, in particular where the restrict sites sandwich a gene or the DNA sequence comprising the genes to allow it/them to be specifically excised from the genome and/or replaced. Alternatively the restriction sites may sandwich each gene, for example when there are two transgenes three different restriction sites are required to ensure that the genes can be selectively excised and/or replaced. In one embodiment one or more, for example all the transgenes are in the form a transgene cassette. In one embodiment BY comprises SEQ ID NO: 11. In one embodiment SEQ ID NO: 11 is interrupted, for example by a transgene. In embodiment SEQ ID NO: 11 is uninterrupted. In one embodiment B_{Y} does not comprise a restriction site. In one embodiment B_{Y} is a bond. In one embodiment B_{Y} comprises or consists of one or more transgenes.

In one embodiment B_{X} and B_{Y} each comprises a restriction site, for example 1, 2, 3 or 4 restriction sites, such as 1 or 2. In one embodiment B_{X} and B_{Y} each comprises at least one transgene, for example 1 or 2 transgenes. In one embodiment B_{X} and B_{Y} each comprises at least one transgene, for example 1 or 2 transgenes and one or more restriction sites, for example 2 or 3 restriction sites, in particular where the restriction sites sandwich a gene or the DNA sequence comprising the genes to allow it to be specifically excised from the genome and/or replaced. Alternatively the restriction sites may sandwich each gene, for example when there are two transgenes three different restriction sites are required to ensure that the genes can be selectively excised and/or replaced. In one embodiment one or more, for example all the transgenes are in the form a transgene cassette. In one embodiment B_{X} and B_{Y} comprises SEQ ID NO: 10 and SEQ ID NO: 11 respectively. In one embodiment B_{X} and B_{Y} do not comprise a restriction site. In one embodiment B_{X} is a bond and B_{Y} is not a bond. In one embodiment BY is a bond and BX is not a bond.

In one embodiment the transgene is located in B_{X}. In one embodiment the transgene or transgene cassette is located in B_{Y}. In one embodiment a transgene or transgene cassette is located in B_{X} and B_{Y}, for example the transgenes may be the same or different, in each location.

Advantageously, the transgene in the present virus constructs is/are inserted in a location that is removed from the early genes because this reduces the likelihood of affecting virus gene expression or speed of replication.

In one independent aspect there is provided a replication competent oncolytic adenovirus of serotype 11 or virus-derivative thereof wherein the fibre, hexon and capsid are serotype 11, wherein the virus genome comprises a DNA sequence encoding a therapeutic antibody or antibody-binding fragment, said DNA sequence under the control of a promoter endogenous to the adenovirus selected from consisting of E4 and the major late promoter, such that the transgene does not interfere with virus replication, for example wherein the DNA sequence encoding the therapeutic antibody or antibody-binding fragment is under the control of the E4 promoter or alternatively under the control of the major late promoter, in particular wherein the DNA sequence encoding an antibody or antibody-binding fragment in located after L5 in the virus genome sequence (i.e. towards the 3′ end of the virus sequence). Advantageously using an endogenous promoter maximises the amount of space available for inserting transgenes.

Advantageously, when under the control of these promoters the virus remains replication competent and is also able to express the antibody as a full length antibody or a suitable binding fragment or other protein. Thus the antibody or other protein of choice will be expressed by the cancer cell. Employing an endogenous promoter may be advantageous because it reduces the size of the transgene cassette that needs to be incorporated to express the antibody, fragment or other protein, i.e. the cassette can be smaller because no exogenous promoter needs to be included.

Employing an endogenous promoter in the virus may also be advantageous in a therapeutic context because the transgene is only expressed when the virus is replicating as opposed to a constitutive exogenous promoter which will continually transcribe the transgene and may lead to an inappropriate concentration of the antibody or fragment.

In one embodiment expression of the antibody or fragment is under the control of the major late promoter.

In one embodiment the expression of the antibody or fragment is under the control of the E4 promoter.

In one independent aspect there is provided a replication competent oncolytic adenovirus of serotype 11 or virus-derivative thereof wherein the fibre, hexon and capsid are serotype 11, wherein the virus genome comprises a DNA sequence encoding a therapeutic antibody or antibody-binding fragment located in a part of the virus genome which is expressed late in the virus replication cycle and such that the transgene does not interfere with virus replication, wherein said DNA sequence under the control of a promoter exogenous to the adenovirus, for example wherein the DNA sequence encoding the therapeutic antibody or antibody-binding fragment is under the control of the CMV promoter, in particular the DNA sequence encoding an antibody or antibody-binding fragment is located after L5 in the virus genome sequence (i.e. towards the end of the 3′ end of the virus sequence).

Employing an exogenous promoter may be advantageous because it can strongly and constitutively express the antibody or fragment, which may be particularly useful in some situations, for example where the patient has very pervasive cancer.

In one embodiment expression of the antibody or fragment is under the control of a CMV promoter.

In one embodiment the exogenous promoter is associated with this DNA sequence, for example is part of the expression cassette encoding the antibody or fragment.

In one embodiment the DNA sequence encoding the antibody or fragment is located after the L5 gene in the virus sequence. Advantageously, the present inventors have established that a variety of transgenes can be inserted into B_{X} and/or B_{Y} under the control of an exogenous or endogenous promoter, without adversely affecting the life cycle of the virus or the stability of the vector.

In one embodiment the transgene is part of a transgene cassette comprising at least one coding sequence (i.e. at least one transgene) and optionally one or more elements independently selected from:

i. a regulator of gene expression, such as an exogenous promoter or splice acceptor;

ii. an internal ribosome entry (IRES) DNA sequence;

iii. a DNA sequence encoding a high self-cleavage efficiency 2A peptide;

iv. a DNA sequence encoding a polyadenylation sequence, and

v. combinations of the same.

Thus in one embodiment the transgene cassette comprises i) or ii) or iii) or iv).

In one embodiment the transgene cassette comprises i) and ii), or i) and iii), or i) and iv), or ii) and iii), or ii) and iv), or iii) and iv).

In one embodiment the transgene cassette comprises i) and ii) and iii), or i) and ii) and iv), or i) and iii) and iv), or ii) and iii) and iv).

In one embodiment the transgene cassette comprises i) and ii) and iii) and iv).

In one embodiment the transgene or transgene cassette comprises a Kozak squence, which assists in the translation of mRNA, for example at the start of a protein coding sequence.

retains the function of the ITR when incorporated into an adenovirus in an appropriate location. In one embodiment the 5′ITR comprises or consists of the sequence from about 1 bp to 138 bp of SEQ ID NO: 12 or a sequence 90, 95, 96, 97, 98 or 99% identical thereto along the whole length, in particular the sequence consisting of from about 1 bp to 138 bp of SEQ ID NO: 12.

The 3′ITR as employed herein refers to part or all of an ITR from 3′ end of an adenovirus which retains the function of the ITR when incorporated into an adenovirus in an appropriate location. In one embodiment the 3′ITR comprises or consists of the sequence from about 32189 bp to 32326 bp of SEQ ID NO: 12 or a sequence 90, 95, 96, 97, 98 or 99% identical thereto along the whole length, in particular the sequence consisting of from about 32189 bp to 32326 bp of SEQ ID NO: 12.

B₁ as employed herein refers to the DNA sequence encoding: part or all of an E1A from an adenovirus, part or all of the E1B region of an adenovirus, and independently part or all of E1A and E1B region of an adenovirus.

When B₁ is a bond then E1A and E1B sequences will be omitted from the virus. In one embodiment B₁ is a bond and thus the virus is a vector.

In one embodiment B₁ further comprises a transgene. It is known in the art that the E1 region can accommodate a transgene which may be inserted in a disruptive way into the E1 region (i.e. in the “middle” of the sequence) or part or all of the E1 region may be deleted to provide more room to accommodate genetic material.

E1A as employed herein refers to the DNA sequence encoding part or all of an adenovirus E1A region. The latter here is referring to the polypeptide/protein E1A. It may be mutated such that the protein encoded by the E1A gene has conservative or non-conservative amino acid changes, such that it has: the same function as wild-type (i.e. the corresponding non-mutated protein); increased function in comparison to wild-type protein; decreased function, such as no function in comparison to wild-type protein; or has a new function in comparison to wild-type protein or a combination of the same as appropriate.

E1B as employed herein refers to the DNA sequence encoding part or all of an adenovirus E1B region (i.e. polypeptide or protein), it may be mutated such that the protein encoded by the E1B gene/region has conservative or non-conservative amino acid changes, such that it has: the same function as wild-type (i.e. the corresponding non-mutated protein); increased function in comparison to wild-type protein; decreased function, such as no function in comparison to wild-type protein; or has a new function in comparison to wild-type protein or a combination of the same as appropriate.

Thus B₁ can be modified or unmodified relative to a wild-type E1 region, such as a wild-type E1A and/or E1B. The skilled person can easily identify whether E1A and/or E1B are present or (part) deleted or mutated.

Wild-type as employed herein refers to a known adenovirus. A known adenovirus is one that has been identified and named, regardless of whether the sequence is available.

In one embodiment B₁ has the sequence from 139 bp to 3932 bp of SEQ ID NO: 12.

B_{A} as employed herein refers to the DNA sequence encoding the E2B-L1-L2-L3-E2A-L4 regions including any non-coding sequences, as appropriate. Generally this sequence will not comprise a transgene. In one embodiment the sequence is substantially similar or identical to a contiguous sequence from a known adenovirus, for example a serotype shown in Table 1, in particular a group B virus, for example Ad3, Ad7, Ad11, Ad14, Ad16, Ad21, Ad34, Ad35, Ad51 or a combination thereof, such as Ad3, Ad11 or a combination thereof. In one embodiment is E2B-L1-L2-L3-E2A-L4 refers to comprising these elements and other structural elements associated with the region, for example B_{A} will generally include the sequence encoding the protein IV2a, for example as follows: IV2A IV2a-E2B-L1-L2-L3-E2A-L4

In one embodiment the E2B region is chimeric. That is, comprises DNA sequences from two or more different adenoviral serotypes, for example from Ad3 and Ad11, such as Ad11p. In one embodiment the E2B region has the sequence from 5068 bp to 10355 bp of SEQ ID NO: 12 or a sequence 95%, 96%, 97%, 98% or 99% identical thereto over the whole length.

In one embodiment the E2B in component BA comprises the sequences shown in SEQ ID NO: 47 (which corresponds to SEQ ID NO: 3 disclosed in WO2005/118825).

In one embodiment B_{A} has the sequence from 3933 bp to 27184 bp of SEQ ID NO: 12.

E3 as employed herein refers to the DNA sequence encoding part or all of an adenovirus E3 region (i.e. protein/polypeptide), it may be mutated such that the protein encoded by the E3 gene has conservative or non-conservative amino acid changes, such that it has the same function as wild-type (the corresponding unmutated protein); increased function in comparison to wild-type protein; decreased function, such as no function in comparison to wild-type protein or has a new function in comparison to wild-type protein or a combination of the same, as appropriate.

In one embodiment the E3 region is form an adenovirus serotype given in Table 1 or a combination thereof, in particular a group B serotype, for example Ad3, Ad7, Ad11 (in particular Ad11p), Ad14, Ad16, Ad21, Ad34, Ad35, Ad51 or a combination thereof, such as Ad3, Ad11 (in particular Ad11p) or a combination thereof.

In one embodiment the E3 region is partially deleted, for example is 95%, 90%, 85%, 80%, 75%, 70%, 65%, 60%, 55%, 50%, 45%, 40%, 35%, 30%, 25%, 20%, 15%, 10%, 5% deleted.

In one embodiment B2 is a bond, wherein the DNA encoding the E3 region is absent.

In one embodiment the DNA encoding the E3 region can be replaced or interrupted by a transgene. As employed herein “E3 region replaced by a transgene as employed herein includes part or all of the E3 region is replaced with a transgene.

In one embodiment the B2 region comprises the sequence from 27185 bp to 28165 bp of SEQ ID NO: 12.

In one embodiment B2 consists of the sequence from 27185 bp to 28165 bp of SEQ ID NO: 12.

B_{X} as employed herein refers to the DNA sequence in the vicinity of the 5′ end of the L5 gene in B_{B}. In the vicinity of or proximal to the 5′ end of the L5 gene as employed herein refers to: adjacent (contiguous) to the 5′ end of the L5 gene or a non-coding region inherently associated herewith i.e. abutting or contiguous to the 5′ prime end of the L5 gene or a non-coding region inherently associated therewith. Alternatively, in the vicinity of or proximal to may refer to being close the L5 gene, such that there are no coding sequences between the BX region and the 5′ end of L5 gene.

Thus in one embodiment B_{X} is joined directly to a base of L5 which represents, for example the start of a coding sequence of the L5 gene.

Thus in one embodiment B_{X} is joined directly to a base of L5 which represents, for example the start of a non-coding sequence, or joined directly to a non-coding region naturally associated with L5. A non-coding region naturally associated L5 as employed herein refers to part of all of a non-coding regions which is part of the L5 gene or contiguous therewith but not part of another gene.

In one embodiment B_{X} comprises the sequence of SEQ ID NO: 10. This sequence is an artificial non-coding sequence wherein a DNA sequence, for example comprising a transgene (or transgene cassette), a restriction site or a combination thereof may be inserted therein. This sequence is advantageous because it acts as a buffer in that allows some flexibility on the exact location of the transgene whilst minimising the disruptive effects on virus stability and viability.

The insert(s) can occur anywhere within SEQ ID NO: 10 from the 5′ end, the 3′ end or at any point between bp 1 to 201, for example between base pairs 1/2, 2/3, 3/4, 4/5, 5/6, 6/7, 7/8, 8/9, 9/10, 10/11, 11/12, 12/13, 13/14, 14/15, 15/16, 16/17, 17/18, 18/19, 19/20, 20/21, 21/22, 22/23, 23/24, 24/25, 25/26, 26/27, 27/28, 28/29, 29/30, 30/31, 31/32, 32/33, 33/34, 34/35, 35/36, 36/37, 37/38, 38/39, 39/40, 40/41, 41/42, 42/43, 43/44, 44/45, 45/46, 46/47, 47/48, 48/49, 49/50, 50/51, 51/52, 52/53, 53/54, 54/55, 55/56, 56/57, 57/58, 58/59, 59/60, 60/61, 61/62, 62/63, 63/64, 64/65, 65/66, 66/67, 67/68, 68/69, 69/70, 70/71, 71/72, 72/73, 73/74, 74/75, 75/76, 76/77, 77/78, 78/79, 79/80, 80/81, 81/82, 82/83, 83/84, 84/85, 85/86, 86/87, 87/88, 88/89, 89/90, 90/91, 91/92, 92/93, 93/94, 94/95, 95/96, 96/97, 97/98, 98/99, 99/100, 100/101, 101/102, 102/103, 103/104, 104/105, 105/106, 106/107, 107/108, 108/109, 109/110, 110/111, 111/112, 112/113, 113/114, 114/115, 115/116, 116/117, 117/118, 118/119, 119/120, 120/121, 121/122, 122/123, 123/124, 124/125, 125/126, 126/127, 127/128, 128/129, 129/130, 130/131, 131/132, 132/133, 133/134, 134/135, 135/136, 136/137, 137/138, 138/139, 139/140, 140/141, 141/142, 142/143, 143/144, 144/145, 145/146, 146/147, 147/148, 148/149, 150/151, 151/152, 152/153, 153/154, 154/155, 155/156, 156/157, 157/158, 158/159, 159/160, 160/161, 161/162, 162/163, 163/164, 164/165, 165/166, 166/167, 167/168, 168/169, 169/170, 170/171, 171/172, 172/173, 173/174, 174/175, 175/176, 176/177, 177/178, 178/179, 179/180, 180/181, 181/182, 182/183, 183/184, 184/185, 185/186, 186/187, 187/188, 189/190, 190/191, 191/192, 192/193, 193/194, 194/195, 195/196, 196/197, 197/198, 198/199, 199/200 or 200/201.

In one embodiment B_{X} comprises SEQ ID NO: 10 with a DNA sequence inserted between bp 27 and bp 28 or a place corresponding to between positions 28192 bp and 28193 bp of SEQ ID NO: 12.

In one embodiment the insert is a restriction site insert. In one embodiment the restriction site insert comprises one or two restriction sites. In one embodiment the restriction site is a 19 bp restriction site insert comprising 2 restriction sites. In one embodiment the restriction site insert is a 9 bp restriction site insert comprising 1 restriction site. In one embodiment the restriction site insert comprises one or two restriction sites and at least one transgene, for example one or two transgenes. In one embodiment the restriction site is a 19 bp restriction site insert comprising 2 restriction sites and at least one transgene, for example one or two transgenes. In one embodiment the restriction site insert is a 9 bp restriction site insert comprising 1 restriction site and at least one transgene, for example one, two or three transgenes, such as one or two. In one embodiment two restriction sites sandwich one or more, such as two transgenes (for example in a transgene cassette). In one embodiment when B_{X} comprises two restrictions sites the said restriction sites are different from each other. In one embodiment said one or more restrictions sites in B_{X} are non-naturally occurring in the particular adenovirus genome into which they have been inserted. In one embodiment said one or more restrictions sites in B_{X} are different to other restrictions sites located elsewhere in the adenovirus genome, for example different to naturally occurring restrictions sites and/or restriction sites introduced into other parts of the genome, such as a restriction site introduced into B_{Y}. Thus in one embodiment the restriction site or sites allow the DNA in the section to be cut specifically.

Advantageously, use of “unique” restriction sites provides selectivity and control over the where the virus genome is cut, simply by using the appropriate restriction enzyme.

Cut specifically as employed herein refers to where use of an enzyme specific to the restriction sites cuts the virus only in the desired location, usually one location, although occasionally it may be a pair of locations. A pair of locations as employed herein refers to two restrictions sites in proximity of each other that are designed to be cut by the same enzyme (i.e. cannot be differentiated from each other).

In one embodiment the restriction site insert is SEQ ID NO: 55.

In one embodiment B_{X} has the sequence from 28166 bp to 28366 bp of SEQ ID NO: 12.

In one embodiment B_{X} is a bond.

B_{B} as employed herein refers to the DNA sequence encoding the L5 region. As employed herein the L5 region refers to the DNA sequence containing the gene encoding the fibre polypeptide/protein, as appropriate in the context. The fibre gene/region encodes the fibre protein which is a major capsid component of adenoviruses. The fibre functions in receptor recognition and contributes to the adenovirus' ability to selectively bind and infect cells.

In viruses of the present disclosure the fibre can be from any adenovirus serotype and adenoviruses which are chimeric as result of changing the fibre for one of a different serotype are known. In one embodiment the fibre is from a group B virus, in particular Ad11, such as Ad11p.

In one embodiment B_{B} has the sequence from 28367 bp to 29344 bp of SEQ ID NO: 12.

DNA sequence in relation to B_{Y} as employed herein refers to the DNA sequence in the vicinity of the 3′ end of the L5 gene of B_{B}. In the vicinity of or proximal to the 3′ end of the L5 gene as employed herein refers to: adjacent (contiguous) to the 3′ end of the L5 gene or a non-coding region inherently associated therewith i.e. abutting or contiguous to the 3′ prime end of the L5 gene or a non-coding region inherently associated therewith (i.e. all or part of an non-coding sequence endogenous to L5). Alternatively, in the vicinity of or proximal to may refer to being close the L5 gene, such that there are no coding sequences between the B_{Y} region and the 3′ end of the L5 gene.

Thus in one embodiment B_{Y} is joined directly to a base of L₅ which represents the “end” of a coding sequence.

Thus in one embodiment B_{Y} is joined directly to a base of L5 which represents the “end” of a non-coding sequence, or joined directly to a non-coding region naturally associated with L5.

Inherently and naturally are used interchangeably herein. In one embodiment B_{Y} comprises the sequence of SEQ ID NO: 11. This sequence is a non-coding sequence wherein a DNA sequence, for example comprising a transgene (or transgene cassette), a restriction site or a combination thereof may be inserted. This sequence is advantageous because it acts a buffer in that allows some flexibility on the exact location of the transgene whilst minimising the disruptive effects on virus stability and viability.

The insert(s) can occur anywhere within SEQ ID NO: 11 from the 5′ end, the 3′ end or at any point between bp 1 to 35, for example between base pairs 1/2, 2/3, 3/4, 4/5, 5/6, 6/7, 7/8, 8/9, 9/10, 10/11, 11/12, 12/13, 13/14, 14/15, 15/16, 16/17, 17/18, 18/19, 19/20, 20/21, 21/22, 22/23, 23/24, 24/25, 25/26, 26/27, 27/28, 28/29, 29/30, 30/31, 31/32, 32/33, 33/34, or 34/35.

In one embodiment B_{Y} comprises SEQ ID NO: 11 with a DNA sequence inserted between positions bp 12 and 13 or a place corresponding to 29356 bp and 29357 bp in SEQ ID NO: 12. In one embodiment the insert is a restriction site insert. In one embodiment the restriction site insert comprises one or two restriction sites. In one embodiment the restriction site is a 19 bp restriction site insert comprising 2 restriction sites. In one embodiment the restriction site insert is a 9 bp restriction site insert comprising 1 restriction site. In one embodiment the restriction site insert comprises one or two restriction sites and at least one transgene, for example one or two or three transgenes, such as one or two transgenes. In one embodiment the restriction site is a 19 bp restriction site insert comprising 2 restriction sites and at least one transgene, for example one or two transgenes. In one embodiment the restriction site insert is a 9 bp restriction site insert comprising 1 restriction site and at least one transgene, for example one or two transgenes. In one embodiment two restriction sites sandwich one or more, such as two transgenes (for example in a transgene cassette). In one embodiment when B_{Y} comprises two restrictions sites the said restriction sites are different from each other. In one embodiment said one or more restrictions sites in B_{Y} are non-naturally occurring (such as unique) in the particular adenovirus genome into which they have been inserted. In one embodiment said one or more restrictions sites in B_{Y} are different to other restrictions sites located elsewhere in the adenovirus genome, for example different to naturally occurring restrictions sites or restriction sites introduced into other parts of the genome, such as B_{X}. Thus in one embodiment the restriction site or sites allow the DNA in the section to be cut specifically.

In one embodiment the restriction site insert is SEQ ID NO: 54.

In one embodiment B_{Y} has the sequence from 29345 bp to 29379 bp of SEQ ID NO: 12.

In one embodiment B_{Y} is a bond.

In one embodiment the insert is after bp 12 in SEQ ID NO: 11.

In one embodiment the insert is at about position 29356 bp of SEQ ID NO: 12.

In one embodiment the insert is a transgene cassette comprising one or more transgenes, for example 1, 2 or 3, such as 1 or 2.

E4 as employed herein refers to the DNA sequence encoding part or all of an adenovirus E4 region (i.e. polypeptide/protein region), which may be mutated such that the protein encoded by the E4 gene has conservative or non-conservative amino acid changes, and has the same function as wild-type (the corresponding non-mutated protein); increased function in comparison to wild-type protein; decreased function, such as no function in comparison to wild-type protein or has a new function in comparison to wild-type protein or a combination of the same as appropriate.

In one embodiment the E4 region is partially deleted, for example is 95%, 90%, 85%, 80%, 75%, 70%, 65%, 60%, 55%, 50%, 45%, 40%, 35%, 30%, 25%, 20%, 15%, 10% or 5% deleted. In one embodiment the E4 region has the sequence from 32188 bp to 29380 bp of SEQ ID NO: 12.

In one embodiment B3 is a bond, i.e. wherein E4 is absent.

In one embodiment B3 has the sequence consisting of from 32188 bp to 29380 bp of SEQ ID NO: 12.

As employed herein number ranges are inclusive of the end points.

The skilled person will appreciate that the elements in the formulas herein, such as formula (I) are contiguous and may embody non-coding DNA sequences as well as the genes and coding DNA sequences (structural features) mentioned herein. In one or more embodiments the formulas of the present disclosure are attempting to describe a naturally occurring sequence in the adenovirus genome. In this context it will be clear to the skilled person that the formula is referring to the major elements characterising the relevant section of genome and is not intended to be an exhaustive description of the genomic stretch of DNA.

E1A, E1B, E3 and E4 as employed herein each independently refer to the wild-type and equivalents thereof, mutated or partially deleted forms of each region as described herein, in particular a wild-type sequence from a known adenovirus.

“Insert” as employed herein refers to a DNA sequence that is incorporated either at the 5′ end, the 3′ end or within a given DNA sequence reference segment such that it interrupts the reference sequence. The latter is a reference sequence employed as a reference point relative to which the insert is located. In the context of the present disclosure inserts generally occur within either SEQ ID NO: 10 or SEQ ID NO: 11. An insert can be either a restriction site insert, a transgene cassette or both. When the sequence is interrupted the virus will still comprise the original sequence, but generally it will be as two fragments sandwiching the insert.

In one embodiment the transgene or transgene cassette does not comprise a non-biased inserting transposon, such as a Tn7 transposon or part thereof. Tn7 transposon as employed herein refers to a non-biased insertion transposon as described in WO2008/080003.

A continuous process of manufacture as employed herein is a process for the manufacture of a virus as defined according to the present disclosure, in particular such that the virus produced by each cell is increased in comparison to non-continuous process, for example where the virus particles produced at at least one or at at least two time points in the process is 50,000 per cell or greater, for example a virus described herein, such as replication competent chimeric oncolytic adenovirus wherein the virus has two or more replication cycles.

A continuous process is the opposite of a discrete culture wherein the cells after infection are not supplemented with additional cells, and cells are, for example lysed to harvest the replicated virus, or cells are discarded after a single virus replication cycle and recovery of the virus therefrom. As part of the process of the present disclosure, virus-containing cell-free supernatant may be harvested multiple times or continuously for downstream purification of virus. In one embodiment the harvesting is not at the end of the period of cell culturing.

In one embodiment the virus particles produced per cell at one or more time points is at least 75,000, for example 80,000; 90,000; 100,000; 150,000; 175,000; 180,000 or 195,000.

The virus yields per cell in these ranges are achieved by selecting the relevant the parameters for the given virus. The parameters that important for achieving these yields are starting seed density, multiplicity of infection (MOI), changing the media and the duration of the process. Whilst not wishing to be bound by theory is believed that controlling these parameters allows control over the process in relation to yield and may also provide control over where the virus product is located i.e. in the supernatant, in the cell or both.

Low multiplicity of infection as employed herein refers to a multiplicity of infection of less than 20, such as 19, 18, 17, 16, 15, 14, 13, 12.5, 12, 11, 10, 9, 8, 7, 6 or 5.

A high multiplicity of infection as employed herein refers to a multiplicity of infection of 45 or higher, such as 46, 47, 48, 49, 50, 51, 52, 53, 54, 55 or higher such as 100.

Low seed density as employed herein refers to a seed density of 2×10⁶ or less, such as 1.9, 1.8, 1.7, 1.6, 1.5, 1.4, 1.3, 1.2, 1.1, 1.0, 0.9, 0.8, 0.7, 0.6 or 0.5×10⁶ or less.

High seed density as employed herein is 3.5×10⁶ or greater, for example 3.6, 3.7, 3.8, 3.9, 4.0, 4.1, 4.2, 4.3, 4.4, 4.5×10⁶ or greater.

In one embodiment of the process the mammalian cells are infected with a starting concentration of virus of 1-9×10⁴ vp/ml or greater, such as 1-9×10⁵, 1-9×10⁶, 1-9×10⁷, 1-9×10⁸, 1-9×10⁹, in particular 1-5×10⁶ vp/ml or 2.5-5×10⁸ vp/ml.

In one embodiment the mammalian cells are infected with a starting seed density of 1-4×10⁶ vp/ml, such as 1, 1.1, 1.2, 1.3, 1.4, 1.5, 1.6, 1.7, 1.8, 1.9, 2, 2.5, 3.0 or 4×10⁶ vp/ml.

In one embodiment of the process the mammalian cells are infected at a starting concentration of 1×10⁶ vp/ml at about 1 to 200 ppc, for example 40 to 120 ppc, such as 50 ppc.

Ppc as employed herein refers to the number of viral particles per cell. Ppc and multiplicity of infection are employed interchangeably herein.

In one embodiment the viruses, such as chimeric oncolytic adenovirus, during culture is at concentration in the range 20 to 150 particles per cell (ppc), such as 40 to 100 ppc, in particular 50 ppc. This concentration is a concentration during the culturing processes as opposed to the starting seed density. Lower values of virus concentrations, for example less than 100 ppc, such as 50 ppc, in particular 20 or less such as 15, 14, 13, 12.5, 12, 11 or 10 may be advantageous. Advantages may include one or more of the following properties increased cell viability compared to cultures with higher virus concentrations, particularly when cell viability is measured before harvesting, and increased levels of virus particle production per cell.

In one embodiment the number of cells in the culture is adjusted to correspond to the levels of virus in the culture, for example cells are added to the culture (such as cells in a stationary phase) or cell growth of the culture is adjusted to provide a cell number that maintain the multiplicity of infection (MOI) about constant.

Virus production also depends on cell density. In one embodiment the cell density is in the range 1 to 10 million cells/ml, including 2 to 10 million cells/ml.

“towards the end of the process” as employed herein refers to the last 40% of the time for which the process is running, for example from about 60 hours post infection, such as 65, 70, 71, 72, 73, 74, 75, 76, 77, 78, 79, 80, 81, 82, 83, 84, 85, 86, 87, 88, 89 or more.

“Viable cells for virus infection and production are maintained in the vessel at a level suitable for replicating the virus” as employed herein refers to maintaining viable cells at a level which is generating desirable, useful, normal yields of virus.

Viable cells are cells which are capable of being infected by virus and/or are capable of supporting replicating virus, in particular healthy cells capable of being infected by virus and/or are capable of supporting replicating virus.

Low cell viability can result in cell lysis which may expose the cell to enzymes or proteins, which with time may attack the virus. However, in a dynamic process such as cell culturing a percentage, such as a small percentage of cells may be lysed. This does not generally cause significant problems in practical terms.

A non-viable cells will be dead, lysed, inactive in respect of viral replication.

A leaky cell is one which has a membrane with increased permeability.

Interestingly cells which are added the culture during the manufacturing process appear to develop increased permeability relatively rapidly, for example within about 24 hours, i.e. where they are stained by a viability dye. Having said that this does not appear to be a disadvantage because high levels of virus particles including infectious viral particles are still produced.

Methods of testing cell viability are known to those skilled in the art and include taking a sample of cells and testing them with a viability stain that penetrates and stains cell which are non-viable and also penetrate leaky cells.

Cell viability assays may be based on one or more of the following techniques:
1. Cytolysis or membrane leakage assays: This category includes the lactate dehydrogenase assay, a stable enzyme common in all cells which can be readily detected when cell membranes are no longer intact. Examples include Propidium iodide, Trypan blue, and 7-Aminoactinomycin D.
2. Mitochondrial activity or caspase assays: Resazurin and Formazan (MTT/XTT) can assay for various stages in the apoptosis process that foreshadow cell death.
3. Functional assays: Assays of cell function will be highly specific to the types of cells being assayed. For example, motility is a widely used assay of sperm cell function. Fertility can be used to assay gamete survival, in general. Red blood cells have been assayed in terms of deformability, osmotic fragility, hemolysis, ATP level, and hemoglobin content.
4. Genomic and proteomic assays: Cells can be assayed for activation of stress pathways using DNA microarrays and protein chips.

A common list of tests employed to assay cell viability include: ATP test, Calcein AM, Clonogenic assay, Ethidium homodimer assay, Evans blue, Fluorescein diacetate hydrolysis/Propidium iodide staining (FDA/PI staining), Flow cytometry, Formazan-based assays (MTT/XTT), Green fluorescent protein, Lactate dehydrogenase (LDH), Methyl violet, Propidium iodide, DNA stain that can differentiate necrotic, apoptotic and normal cells, Resazurin, Trypan Blue, a living-cell exclusion dye (dye only crosses cell membranes of dead cells), and TUNEL assay.

Thus the viability of cells can be tested by cell staining with, for example Trypan blue (and light microscopy) or 7-amino-actinomycin D, vital dye emitting at 670 nm (or ViaProbe a commercial ready-to-use solution of 7AAD) and flow cytometry, employing a technique known to those skilled in the art. Where the stain penetrates into the cells the cells are considered not viable. Cells which do not take up dye are considered viable. An exemplary method may employ about 5 μL of 7AAD and about 5 μL of Annexin-V (a phospholipid-binding protein which binds to external phospholipid phosphatidylserine exposed during apoptosis) per approximate 1004 of cells suspension. This mixture may be incubated at ambient temperature for about 15 minutes in the absence of light. The analysis may then be performed employing flow cytometry. See for example MG Wing, AMP Montgomery, S. Songsivilai and JV Watson. An Improved Method for the Detection of Cell Surface Antigens in Samples of Low Viability using Flow Cytometry. J Immunol Methods 126: 21-27 1990.

In one embodiment oxygen uptake or oxygen transfers is used to evaluate the viability of the cells. This may be a more appropriate method of analysing the viability of cells because the leaky cells which are still capable of replicating virus may be stained with reagents such as Trypan blue etc. Thus this method may be used to differential dead/lysed cells vs leaky cells.

Surprisingly, the present inventors have found that, when employing some embodiment s of the process, the cells maintain high viability (such as 80 to 90% viability in this context is not stained with a viability dye) post-infection for over the periods described herein for continuous manufacture, in particular when there is no addition of fresh cells. Thus in one embodiment the harvesting and process may continue as long as sufficient cells remain viable.

In one embodiment with no media exchange and no addition or replacement of cells supporting virus replication the cell viability is around 50 to 100% during the process, for example 60 to 95% at the 96 hour time point (i.e. 96 hours post-infection) when infected with EnAd, such as 90% viability (i.e. 90% of cells had no staining with a viability dye).

In one embodiment cell viability is around 50 to 100% during the process, for example 60 to 90% at the 96 hour time point (i.e. 96 hours post-infection) when infected with NG76, such as 83% viability (i.e. 83% of cells had no staining with a viability dye), in particular when no fresh cells are added.

In one embodiment cell viability is around 50 to 100% during the process, for example 60 to 90% at the 96 hour time point (i.e. 96 hours post-infection) when infected with NG135, such as 85% viability (i.e. 85% of cells had no staining with a viability dye), in particular when no fresh cells are added.

In one embodiment cell viability is around 50 to 100% during the process, for example 80 to 90% at the 96 hour time point (i.e. 96 hours post-infection) when infected with Ad11. For example 85% viability (i.e. 85% of cells had no staining with a viability dye), in particular when no fresh cells are added.

In one embodiment the culture process comprises one or more cell additions or changes. Cell addition employed herein refers to replenishing some or all of the cells and optionally removing dead cells. Cell change as employed herein refers to removing at least some cells and adding fresh cells

In one embodiment during the period of continuous manufacture non-viable cells are replaced to maintain an ongoing/sustained period of virus replication and ongoing/sustained release of virus into the culture medium, for example virus replication is a continuous (i.e. non-interrupted) although virus replication levels may fluctuate during the process.

In one embodiment the viability of the newly added cells may be the important factor and thus if the cell viability (i.e. as tested by viability staining dye) across the whole cell culture is measured it may in fact be below 50%, provided there are sufficient cells to keep replicate adequate amounts of virus. However, the dye staining does not necessarily differentiate between non-viable cells and leaky cells.

Whilst not wishing to be bound by theory cells which may be leaky also appear to be viable to produce virus.

In some instances 70% or more of the cells employed in the process may be leaky, for example due to the stress exerted on them by the present process. However, this seems to have little impact on the performance of the cells in producing virus. Thus for the purpose of the present process the cells may remain sufficiently viable for producing virus even when a viability dye would stain them. In processes according to the present disclosure where new cells are added to the culture the cell staining with a viability stain may be very high, for example 70% of the cell population in experiments performed in shake flasks even after the addition of the fresh cells. Having said that bioreactor processes are particularly suitable for performing virus manufacturing in mammalian cells and the amount of staining of cells from a bioreactor process may be less.

In one embodiment a process according to the present disclosure is performed in a bioreactor.

An example of a suitable bioreactor system is the iCELLis bioreactor, which is a fully integrated high density bioreactor packed with for example microfibres. This system is particularly suitable for culturing adherent cells. Evenly-distributed media circulation is achieved by a built-in magnetic drive impeller, ensuring low shear stress and high cell viability. The cell culture medium flows through the fixed-bed from the bottom to the top. At the top, the medium falls as a thin film down the outer wall where it takes up O₂ to maintain high K_{L}a. in the bioreactor. This oxygenation, together with a gentle agitation and biomass immobilization, enables the compact iCELLis bioreactor to achieve and maintain high-cell densities, equaling the productivity of much larger stirred tank units. See further details at for example:

http://www.pall.com/main/biopharmaceuticals/product.page?lid=hw7uq21l

An ongoing or sustained period of virus replication is one where the virus has time for more than one replication cycle (i.e. has two or more replication cycles). A replication cycle is where a given virus enters a cell replicates and the virus and/or viral particles are released from the cell and then the virus or the progeny thereof go on to infect a cell or cells and proceed to replicate.

A process for the manufacture of a virus as employed herein is intended to refer to a process wherein the virus is replicated and thus the number of viral particles is increased. In particular the manufacturing is to provide sufficient numbers of viral particles to formulate a therapeutic product, for example in the range 1-9×10⁵ to 1-9×10²⁰ or more particles may be produced, such as in the range of 1-9×10⁸ to 1-9×10¹⁵ viral particles, in particular 1 to 9×10¹⁰ or 1-9×10¹⁵ viral particles may be produced from a 10 L batch.

In one embodiment the yield is about 1-2×10′ vp per litre.

In one embodiment the virus production per cell is 50,000 virus particles or greater, for example independently selected from the group comprising 55,000; 60,000; 65,000; 70,000; 75,000; 80,000; 90,000; 95,000; 100,000; 105,000; 110,000; 115,000; 120,000; 125,000; 130,000; 135,000; 140,000; 145,000; 150,000; 155,000; 160,000; 165,000; 170,000; 175,000; 176,000; 177,000; 178,000; 179,000; 180,000; 181,000; 182,000; 183,000; 184,000; 185,000; 186,000; 187,000; 188,000; 189,000; 190,000; 191,000; 192,000; 193,000; 194,000; 195,000; 196,000; 197,000; 198,000; 199,000; 200,000; 201,000; 202,000 virus particles per cell or greater, at one or more time points.

The period of continuous manufacture as employed herein is simply the duration of a given manufacturing campaign. Generally the processes of the invention will have a period of continuous manufacture, which is 48 hours or greater, for example 70, 80, 84, 86, 90, 96, 100, 108, 120, 124, 128, 132, 144, 156, 168, 180, 192, 204, 216, 228, 240 hours or more (wherein said values are +/−3 hours), for example in the range 144 hours to 240 hours, such as 156, 168, 180, 192, 204, 216, 228 or 240 hours.

In one embodiment the culturing period is in the range 70 to 300 hours, for example 144 to 240 hours, for example 144, 156, 168, 180, 192, 204, 216, 228 or 240 hours.

In one embodiment the continuous manufacturing process is characterised in that the cells are cultured for more than 100 hours.

In one embodiment the continuous manufacturing process of the present disclosure is characterised by a post infection culturing period of up to 100 hours and at least one media change or addition and at least one cell change or cell addition.

In on embodiment the continuous manufacture is in the range 35 to 96 hours post infection.

Advantageously, the viruses of the present disclosure do not appear to degrade, even when the culturing process is extended to 70 hours or more. The degradation of the virus can be checked by assaying the infectivity of the virus. The infectivity of the virus decreases as the viral particles degrade.

Maximum total virus production as employed herein means the total number of viral particles produced per cell and encompasses viral particles in the supernatant and the cell.

In one embodiment the maximum total virus production is achieved at about 40 to 60 hours post-infection and multiples thereof, for example 49, 98, 147 etc hours post-infection.

In one embodiment the maximum total virus production is achieved at about 70 to 90 hours post-infection and multiples thereof, for example 140-180 hours post infection.

In one embodiment maximum total virus yield is achieved at about 60 to 96 hours post infection.

Changing/replacing the media one or more times in the process appears to have a positive and significant impact on virus yield. In one embodiment the media is changed without removing cells. In one embodiment 5 to 100% of the media is changed at at least one time point. In one embodiment media and cells are removed, for example 10, 20, 30, 40, 50, 60, 70, 80% or more of cell suspension (of a cell suspension culture process) is removed and replaced with fresh media and cells.

In one embodiment media is added on one or more occasions during the process. This may be particularly advantageous when the host cells are in an exponential growth phase.

In one embodiment the culture process comprises one or more media changes. This may be beneficial for optimising cell growth, yield or similar. Where a medium change is employed, it may be necessary to recover virus particle from the media being changed. These particles can be combined with the main virus batch to ensure the yield of virus is optimised. Similar techniques may also be employed with the medium of a perfusion process to optimise virus recovery.

In one embodiment the culture process does not include a medium change step. This may be advantageous because no viral particles will be lost and therefore yield may be optimised.

In one embodiment the medium and/or cells are supplements or replenished periodically.

In one embodiment the cells are harvested during the process, for example at a discrete time point or time points or continuously.

Media suitable for culturing mammalian cells includes, but are not limited to, EX-CELL® media from Sigma-Aldrich, such as EX-CELL® 293 serum free medium for HEK293 cells, EX-CELL® ACF CHO media serum free media for CHO cells, EX-CELL® 302 serum free media for CHO cells, EX-CELL CD hydrolysate fusion media supplement, from Lonza RMPI (such as RMPI 1640 with HEPES and L-glutamine, RMPI 1640 with or without L-glutamine, and RMPI 1640 with UltraGlutamine), MEM, DMEM, and SFMII medium.

In one embodiment the media is serum free. This is advantageous because it facilitates registration of the manufacturing process with the regulatory authorities.

The addition of fresh media as employed herein refers to the addition of media where the nutrients and ingredients are at suitable level for supporting cell growth, i.e. not depleted.

Media change as employed herein refers to replacing some or all of the media employed in the process, for example removing some media and adding fresh media.

“Isolating from the media the virus produced from step a) wherein the isolation of virus is not subsequent to a cell lysis step” as employed herein refers to at least one step wherein virus obtained from the process is isolated from the supernatant. The process may also, if appropriate comprise a cell lysis step to recover virus from the cells, for example at the end of the process and/or to recover virus from cells that have been removed from the culture.

“Wherein the isolation is not subsequent to a cell lysis step” as employed herein is intended to refer to the fact the harvesting at at least one time point in manufacturing process does not comprise a specific lysis step. That is to say a step where the conditions are designed to lyse all or most of the cells in the culture, for example does not employ a chemical lysis step, an enzymatic lysis step, a lysis buffer step, a mechanical lysis step or a physical lysis step such as centrifuging or freeze-thawing.

Most as employed herein refers to a large majority, for example 80, 90, 91, 92, 93, 94, 95, 96, 97, 98 or 99%.

In one embodiment after the virus is released from the cells into the supernatant then harvesting is commenced and is performed continuously from that point onwards (for example by recovery from the circulating media) or is harvested at discrete time points, for example 40 to 50 hours, 60 to 70 hours, 80 to 100 hours, 120 to 150 hours, 160 to 200 hours, 200 to 250 or a combination of the above. A combination where a certain amount, but not all, of virus is being removed from the supernatant continuously and at certain time points the amount of virus being removed is increased or decreased.

Harvesting of virus should leave sufficient virus in the culture to continue viral replication. This, for example can be achieved my monitoring the MOI and keeping it within a range described herein.

In one embodiment all the virus is harvested at one time point at the end of the process.

In one embodiment all the virus is not harvested at one time point at the end of the process.

In one embodiment the process of the present disclosure comprises one or more filtration steps. Filtration can be selected as appropriate to retain cells and contaminants thereby allowing virus to pass through the pores of the filter. Alternatively the filtration may be selected to retain the virus and allow the contaminants through. In one embodiment a combination of filtration techniques are employed in the process of the present disclosure.

The virus may be removed from the supernatant by filtration, for example filters may be employed with pores sufficient to allow virus through but retain cells and other cell culture components (contaminants). In one embodiment the filter employed in the range 0.1 to 10 microns, for example 0.2 microns. In one embodiment graduated filtration is employed, for example a 10 micron filter is followed by a smaller filter for example in the range 1 to 5 microns, which is in turn followed by a smaller filter such as 0.2 microns or similar.

In one embodiment diafitration, such as tangential flow filtration, is employed. The size of the filter and conditions can be selected to selectively extract the virus. Examples of filters that may be suitable include TFF membrane 300K NMWC and equivalents thereof.

In one embodiment diafiltration systems having a cut-off of about 300 kDa may be appropriate because this generally allows retention of 90% or more of the virus particles whilst removing contamination.

The supernatant remaining after extraction of the part or all of the virus contained therein can, if appropriate, be further processed to extract more virus and increase the overall yield, can be recycled into the culture system optionally in combination with an amount of fresh media or components thereof, or discarded, or a combination thereof.

In one embodiment fractions (including one fraction) of virus isolated from the cell (as opposed to virus removed from the supernatant) have at least one purification step performed on them before addition to the virus isolated from the supernatant.

In one embodiment fractions (including one fraction) of virus isolated from the cell (as opposed to virus removed from the supernatant) do not have at least one purification step performed on them before addition to the virus isolated from the supernatant.

In one embodiment over 90% of the virus is present in the supernatant at the 48 hour time point and multiples thereof, for example, 91, 92, 93, 94, 95, 96, 97, 98, 99 or 100%, such as 95% or more, particularly 98% or more.

In one embodiment significant amounts of virus are in media post 38 hours. For example, over 50%, particularly over 70% of the virus is in the media post 38 hours and multiples thereof, for example 76, 114, 152, 190, 228 etc.

In one embodiment 50% or more, such as 55, 60, 65, 70, 75% or more of the virus is in the supernatant by about 65 hours.

In one embodiment 70% or more, such as 75, 76, 77, 78, 79, 80% or more of the virus is in the supernatant by about 72 hours.

In one embodiment 80% or more, such as 85, 86, 87, 88, 89, 80% or more of the virus is in the supernatant by about 96 hours.

In one embodiment there is less than 10% detectable virus in the CVL pellet at the 64 hour time point, i.e. post 64 hours, such as 9, 8, 7, 6, 5, 4, 3, 2, 1% detectable virus. CVL as employed herein means the crude viral lysate.

In one embodiment the virus after replication exits the cells about the same time for substantially all the cells, such that there are specific time points when virus in the supernatant peaks. Unless the context indicates otherwise virus replication cycle as employed herein refers to virus peaks in the process, the first viral peak is indicative of the end of the first virus replication cycle and the start of the second virus replication cycle and so on. This profile of process may be achieved by using cells cultured at the same time to ensure they are at the same or approximately the same stage of their life cycle at any given moment in time. Alternatively a range of different cells can be employed to flatten this profile and provide replicated virus on a continuous basis.

The virus levels in the media or supernatant can be monitored by HPLC or other technique.

Generally cells added to the culture will be the same type of cells being employed in the culture.

Cells added to the culture may have been pre-cultured to render them more suitable for addition to the main culture, for example to ensure that they are at a compatible stage of the cell life life-cycle to be mixed with the cells of the main culture.

Advantageously, if the viable cell levels are increased, as virus replication increases the absolute number of virus particles then cells are available for the virus to infect, thereby providing a very efficient process. In one embodiment other properties of the population are changed by changing or adding cells, for example membrane permeability may be increased.

In one embodiment the cells are maintained under conditions established to support and maintain log phase growth during infection and replication of virus. Thus in one embodiment at least a proportion of the cell population is in a logarithmic growth phase at any given time in the process, for example 10 to 80%, such as 20 to 50% of the cells are in a growth phase. Logarithmic growth phase as used herein means cells are proliferating exponentially under growth media conditions where available nutrients, substrates and inhibitory by products released by cells are not limiting factors.

Culturing cells may employ a perfusion culture, fed batch culture, batch culture, a steady state culture, a continuous culture or a combination of one or more of the same as technically appropriate, in particular a perfusion culture.

In one embodiment the process is a perfusion process, for example a continuous perfusion process.

“Derived from” as employed herein refers to, for example where a DNA fragment is taken from an adenovirus or corresponds to a sequence originally found in an adenovirus. This language is not intended to limit how the sequence was obtained, for example a sequence employed in a virus according to the present disclosure may be synthesised.

In one embodiment the derivative has 100% sequence identity over its full length to the original DNA sequence.

In one embodiment the derivative has 95, 96, 97, 98 or 99% identity or similarity to the original DNA sequence.

In one embodiment the derivative hybridises under stringent conditions to the original DNA sequence.

As used herein, “stringency” typically occurs in a range from about Tm (melting temperature)-50° C. (5° below the Tm of the probe) to about 20° C. to 25° C. below Tm. As will be understood by those of skill in the art, a stringent hybridization can be used to identify or detect identical polynucleotide sequences or to identify or detect similar or related polynucleotide sequences. As herein used, the term “stringent conditions” means hybridization will generally occur if there is at least 95%, such as at least 97% identity between the sequences.

As used herein, “hybridization” as used herein, shall include “any process by which a polynucleotide strand joins with a complementary strand through base pairing” (Coombs, J., Dictionary of Biotechnology, Stockton Press, New York, N.Y., 1994).

Advantageously, the present process may simplify downstream processing of the virus because of the lower starting concentration of contaminating DNA or proteins from the cells because a cell lysis step can be avoided. This may result in cost savings because reagents, equipment and time employed in downstream processing may be reduced. It may also result in greater purity with lower end concentrations of contaminating DNA and/or a lower concentration of large fragments of contaminating cellular DNA and proteins.

Furthermore, virus exposure to cell enzymes is minimised by avoiding cell lysis, which minimises the exposure of the virus to potential degradants, such as nucleases from the cell. This may result in higher virus stability and/or potency as measured, for example by infectivity.

Interestingly, after exiting the cells the virus of the present disclosure does not adhere to the cells and so can be readily recovered from the supernatant. This may be a phenomenon which is characteristic of the certain viruses described herein which facilitates the current process. In contrast, wild-type Ad5 is thought to adhere to cells. In fact, results have shown that substantially no wild-type Ad5, viral particles are present in the supernatant the time frames discussed herein.

Whilst not wishing to be bound by theory, in one embodiment the ability to exit the cell and not adhere thereto and/or a rapid viral replication cycle may be associated with the chimeric E2B region and/or deletion of part of the E3 and/or deletion of the E4 region.

Whilst not wishing to be bound by theory, in one embodiment the ability to exit the cell and not adhere thereto and/or a rapid viral replication cycle may be associated with the group B capsid, for example Ad11 capsid.

In one embodiment the lack of adherence to the cells may be related to the hexon and fibre of the oncolytic virus, for example in this respect virus capsids from group B adenoviruses, such as Ad11 may be particularly advantageous for replication competent viruses and chimeric viruses of the present disclosure.

Rapid virus replication cycle as employed herein refers to a cycle complete such that at least some of the virus is excreted into the supernatant in a period 50 hours or less after infection.

In one embodiment known standard systems are employed to the process the viruses prepared by the methods described herein.

In one embodiment, for example where the continuous process is in fact non-stop, i.e. run for very prolonged campaigns, such as week and months as opposed to days, then the culture vessel may be adapted to provide in-line tangential flow filtration of the culture media to avoid build-up of contaminants, such as DNA and cell debris. One possible arrangement is show in FIG. 2, wherein a tangential flow filtration system is provided adjoined to culture vessel. The interface in between the culture vessel and the tangential flow system is shown a flat interface in FIG. 2. However the interface may be any suitable shape, for example the tangential flow filtration may be arranged as jacket around the culture vessel.

The interface with the vessel is a selectively permeable membrane, referred to a tangential flow filter membrane. In one embodiment the membrane is 300K NMWC TFF.

The arrangement is FIG. 2 is only an example of a suitable arrangement, for example media post viral extraction may be recycled into the culture, pumps can be arranged differently.

Thus in one embodiment the tangential flow system associated with the culture vessel removes contamination from the culture. Thus in one embodiment the cell culture vessel comprises a tangential flow interface, for example maintained at a pressure differential to allow removal of cell debris and contaminants from the culture, thereby prolonging the period over which the culture can be maintained.

In one embodiment the tangential flow system associated with the culture vessel removes virus from the culture, and thus can be employed for harvesting virus.

The separation system may remove virus as a primary recovery of virus or secondary recover. The separation system may also have an exit to waste.

In one embodiment the separation system may be absent.

Vessel as employed herein refers to any container suitable for use to culture cells in, for example a culture bag, pot, bottle, cube, tube, bioreactor or similar. In one embodiment the vessel may comprise a gas permeable membrane.

In one embodiment the culture is a suspension culture. In another embodiment the culture is an adherent culture.

Mammalian cells are cell derived from a mammal. In one embodiment the mammalian cells are selected from the group comprising HEK, CHO, COS-7, HeLa, Vero, A549, PerC6 and GMK, in particular HEK293 or A549 cells. In one embodiment the cells added to the culture are quick growing cells, for example 293-F. In one embodiment the cells/cell line employed is not an encapsidating cell line, that is a packaging cell line that provide virus transgenes to facilitate viral replication.

Adherent cells cultivated on, for example polymer spheres (microbeads also referred to as microcarriers) or attached to hollow-fibres can be employed in suspension in stirred tank bioreactors.

At least HEK, CHO and A549 can be rendered adherent cell lines.

Culturing mammalian cells as employed herein refers to the process where cells are grown under controlled conditions ex vivo. Suitable conditions are known to those in the art and may include temperatures such as 37° C. The CO₂ levels may need to be controlled, for example kept at a level of less of 10%, such as 5%. Details of the same are given in the text Culture of Animal Cells: A Manual of Basic Techniques and Specialised Applications Edition Six R. Ian Freshney, Basic Cell Culture (Practical Approach) Second Edition Edited by J. M. Davis.

Usually the cells will be cultured to generate sufficient numbers before infection with the virus of the present disclosure. These methods are known to those skilled in the art or are readily available in published protocols or the literature.

Generally the cells will be cultured on a commercial scale, for example 5 L, 10 L, 15 L, 20 L, 25 L, 30 L, 35 L, 40 L, 45 L, 50 L, 100 L, 200 L, 300 L, 400 L, 500 L, 600 L, 700 L, 800 L, 900, 1000 L or similar. In one embodiment the scale of the continuous process according to the present disclosure is 150 L or less.

The viruses of the present disclosure, such as chimeric oncolytic viruses and/or group B replication competent adenoviruses, have different properties to those of adenoviruses used as vectors such as Ad5, this includes the fact that they can be recovered from the medium without the need for cell lysis over relatively short periods of culture. This is contrary to what was expected in the art because for a long time it was thought that the adenovirus death protein encoded in the E3 region of Ad5 (a group C virus) was required for the virus to exit the cell. Thus, whilst not wishing to be bound by theory, the group B viruses appear to have mechanisms to exit the cell.

Furthermore, the viruses of the present disclosure, such as chimeric oncolytic adenoviruses do not seem to associate or adhere the cells after exiting the same, which also facilitates recovery from the supernatant, in particular when the cell culturing conditions are optimised.

In one embodiment the process is performed at about a temperature below 40° C., for example about 35 to 39° C., such as 37° C.

In one embodiment the process performed with a carbon dioxide concentration less than 10% for example about 4-6% CO₂, such as 5% CO₂.

In one embodiment the process is performed at a pH in the range 6 to 8, such as pH 7.4.

In one embodiment the media containing the virus, such as the chimeric oncolytic viral particles is filtered to remove the cells and provide crude supernatant for further downstream processing.

In one embodiment a tangential flow filter is employed.

In one embodiment medium is filtered employing Millipore's Millistak+® POD system with cellulose based depth filters. Millistak+® depth filter medium is offered in a scalable, disposable format, the Pod Filter System. It is ideal for a wide variety of primary and secondary clarification applications, including cell cultures.

Millistak+® Pod filters are available in three distinct series of media grades in order to meet specific application needs. Millistak+® DE, CE and HC media deliver optimal performance through gradient density matrix as well as positive surface charge properties. In one embodiment the filtration is effected using tangential flow technology, for example employing the Cogent™ M system comprising a Pellicon Mini cassette membrane holder, pressure sensors, 10 litre recycle tank with mixer, retentate flow meter, weigh scale, feed pump, transfer pump, piping and valves. Control and operation of the system is manual with an exception of semi-automatic diafiltration/concentration. The operator has manual control of pump speeds, all valves and operational procedures. The virus can also, if desired, be formulated into the final buffer in this step.

In one embodiment the downstream processing comprises of a clarification assembly consisting of an 8 μm capsule filter (Sartopure PP2 8 μm) followed by a 3.0 μm/0.8 μm capsule filter (Sartoclean CA, 3.0 μm/0.8 μm, 0.2 m²) in series.

Thus in one embodiment in the filtration step, concentrated and conditioned adenovirus material is provided in a final or near final formulation.

In one embodiment the process comprises two or more filtration steps.

In one embodiment the downstream processing comprises Millistak+POD system 35 CE and 50 CE cassettes followed by an opticap XL 10 express 0.5/0.2 um membrane filter in series.

In one embodiment the process further comprises a purification step, selected from a CsCl gradient, chromatography step such as size exclusion chromatography, ion-exchange chromatography in particular anion-exchange chromatography, and a combination thereof.

Ion exchange chromatography binds DNA very strongly and typically is the place were any residual DNA is removed. The ion exchange resin/membrane binds both the virus and the DNA and during salt gradient elusion the virus normally elutes off the column first (low salt gradient) and the DNA is eluted at a much higher salt concentration since the interaction of the DNA with the resin is stronger than the virus.

In one embodiment the chromatography step or steps employ monolith technology, for example available from BIA Separations.

In one embodiment Sartobind Q (quaternary amine membrane purification process) is employed as a purification step.

In one embodiment Source Q RESIN is employed in a purification step.

In one embodiment Sartobind Q is employed followed by Source Q RESIN in downstream processing of the isolated virus.

In one embodiment Sartobind Q is employed followed by CIM® monolithic columns (CIM-QA; quaternary amine membrane purification process) in downstream processing of the isolated virus.

In one embodiment Source Q is employed in the purification step.

During chromatography stages of the manufacturing process (i.e. capture and polishing steps) several columns may be connected in series such that the first column can be over loaded beyond the binding capacity without loss of material since the product that breaks through from the first column is captured on the second column in the series. The principle of continuous multicolumn manufacturing process thus creates a (simulated) movement of the columns in the opposite direction to the product flow referred to as the countercurrent chromatography process.

Three columns or more may be required for the countercurrent chromatography process, such that the first column (which has been over loaded beyond the binding capacity) has sufficient time to be processed through the wash step, elution, regeneration and re-equilibration step and then brought back in line to capture product whilst another (product saturated) column in the series undergoes the wash to re-equilibration steps outlined above. The columns are cycled many times throughout each purification run, usually for the life time of the chromatography media.

In one embodiment after purification the virus prepared contains less than 80 ng/mL of contaminating DNA, for example between 60 ng/mL and 10 ng/mL.

In one embodiment substantially all the contaminating DNA fragments are 700 base pairs or less, for example 500 bp or less, such as 200 bp or less.

In one embodiment the residual host cell protein content in the purified virus product in 20 ng/mL or less, for example 15 ng/mL or less, in particular when measured by an ELISA assay.

In one embodiment the residual tween in the purified virus product is 0.1 mg/mL or less, such as 0.05 mg/mL or less.

Benzonase (Merck), 100 U/ml, is used to digest host cell DNA. Benzonase treatment is done for 30 min in +37° C. Benzonase is stopped with high salt incubation for 1 hour at RT. The use of benzonase to degrade cellular DNA may also be avoided or reduced if desired, which may be advantageous. In particular, removal of the benzonase and testing to show the absence of residual benzonase can be avoided. In one embodiment benzonase is not employed in the present process.

In one embodiment the residual benzonase content in the purified virus product is 1 ng/mL or less, such as 0.5 ng/mL or less.

In the context of the present application, medium and media may be used interchangeably.

Oncolytic viruses are those which preferentially infect cancer cells and hasten cell death, for example by lysis of same, or selectively replicate in the cancer cells.

Viruses which preferentially infect cancer cells are viruses which show a higher rate of infecting cancer cells when compared to normal healthy cells.

A chimeric adenovirus of the present disclosure can be evaluated for its preference for a specific tumor type by examination of its lytic potential in a panel of tumor cells, for example colon tumor cell lines include HT-29, DLD-1, LS174T, LS1034, SW403, HCT116, SW48, and Colo320DM. Any available colon tumor cell lines may be employed for such an evaluation.

Prostate cell lines include DU145 and PC-3 cells. Pancreatic cell lines include Panc-1 cells. Breast tumor cell lines include MDA231 cell line and ovarian cell lines include the OVCAR-3 cell line. Lung cancer cell lines include A549. Hemopoietic cell lines include, but are not limited to, the Raji and Daudi B-lymphoid cells, K562 erythroblastoid cells, U937 myeloid cells, and HSB2 T-lymphoid cells. Other available tumor cell lines may be equally useful.

Oncolytic viruses including those which are non-chimeric, for example Ad11, such as Ad11p can similarly be evaluated in these cell lines.

In one embodiment the chimeric oncolytic virus is apoptotic, that is hastens programmed cell death.

In one embodiment the chimeric oncolytic virus is cytolytic. The cytolytic activity of chimeric oncolytic adenoviruses of the disclosure can be determined in representative tumor cell lines and the data converted to a measurement of potency, for example with an adenovirus belonging to subgroup C, preferably Ad5, being used as a standard (i.e. given a potency of 1). A suitable method for determining cytolytic activity is an MTS assay (see Example 4, FIG. 2 of WO2005/118825 incorporated herein by reference).

In one embodiment the chimeric oncolytic adenovirus of the present disclosure causes cell necrosis.

In one embodiment the chimeric oncolytic virus has an enhanced therapeutic index for cancer cells.

Therapeutic index” or “therapeutic window” refers to a number indicating the oncolytic potential of a given adenovirus which may be determined by dividing the potency of the chimeric oncolytic adenovirus in a relevant cancer cell line by the potency of the same adenovirus in a normal (i.e. non-cancerous) cell line.

In one embodiment the chimeric oncolytic virus has an enhanced therapeutic index in one or more cancer cells selected from the group comprising colon cancer cells, breast cancer cells, head and neck cancers, pancreatic cancer cells, ovarian cancer cells, hemopoietic tumor cells, leukemic cells, glioma cells, prostate cancer cells, lung cancer cells, melanoma cells, sarcoma cells, liver cancer cells, renal cancer cells, bladder cancer cells and metastatic cancer cells.

A chimeric oncolytic adenovirus as employed herein refers to an adenovirus comprising an E2B region which has DNA sequence derived from at least two distinct adenovirus serotypes and wherein the virus is oncolytic.

There are currently about 56 adenovirus serotypes. Table 1 shows the division of adenovirus serotypes:

| Subgroup | Adenoviral Serotype |
|---|---|
| A | 12, 18, 31 |
| B | 3, 7, 11, 14, 16, 21, 34, 35, 50, 55 |
| C | 1, 2, 5, 6 |
| D | 8-10, 13, 15, 17, 19, 20, 22-30, 32, 33, |
| | 36-39, 42-51, 53, 54, 56 |
| E | 4 |
| F | 40, 41 |
| G | 52 |

The E2B region is a known region in adenoviruses and represents about 18% of the viral genome. It is thought to encode protein IVa2, DNA polymerase and terminal protein. In the Slobitski strain of Ad11 (referred to as Ad11p) these proteins are encoded at positions 5588-3964, 8435-5067 and 10342-8438 respectively in the genomic sequence and the E2B region runs from 10342-3950. The exact position of the E2B region may change in other serotypes but the function is conserved in all human adenovirus genomes examined to date as they all have the same general organisation.

In one embodiment the virus of the present disclosure, such as a chimeric oncolytic virus has a subgroup B hexon.

In one embodiment the virus of the disclosure, such as a chimeric oncolytic virus has an Ad11 hexon, such as an A11p hexon.

In one embodiment the virus of the disclosure, such as a chimeric oncolytic virus has a subgroup B fibre.

In one the virus of the disclosure, such as a chimeric oncolytic virus has an Ad11 fibre, such as an A11p fibre.

In one embodiment the virus of the disclosure, such as a chimeric oncolytic virus has fibre and hexon proteins from the same serotype, for example a subgroup B adenovirus, such as Ad11, in particular Ad11p.

In one embodiment the virus of the disclosure, such as a chimeric oncolytic virus has fibre, hexon and penton proteins from the same serotype, for example Ad11, in particular Ad11p, for example found at positions 30811-31788, 18254-21100 and 13682-15367 of the genomic sequence of the latter.

A virus of a distinct serotype to a first virus may be from the same subgroup or a different subgroup but will always be from a different serotype. In one embodiment the combinations are as follows in (first Ad serotype: second Ad serotype): AA, AB, AC, AD, AE, AF, AG, BB, BC, BD, BF, BG, CC, CD, CE, CF, CG, DD, DE, DF, DG, EE, EF, EG, FF, FG and GG.

In one embodiment the chimeric E2B region is derived from Ad3 and Ad11 (in particular Ad11p).

In one embodiment the E2B region is the sequence shown in SEQ ID NO: 47 herein.

In one embodiment the virus has a hexon and fibre from a group B adenovirus, for example Ad11 and in particular wherein the virus is ColoAd1.

In one embodiment there is provided isolated purified EnAd wherein the contaminating DNA content is less than 80 ng/mL.

EnAd is disclosed in WO2005/118825 and the full sequence for the virus is provided herein, namely SEQ ID No: 12.

Alternative chimeric oncolytic viruses include OvAd1 and OvAd2, which are SEQ ID NO: 2 and 3 respectively disclosed in WO 2008/080003 and incorporated herein by reference.

In one embodiment the virus of the disclosure, such as the chimeric oncolytic virus of the present disclosure comprises one or more restrictions site into which a transgene. In one embodiment the restriction site is in or is adjacent to a late gene, for example L5.

In one embodiment the virus of the disclosure, such as the chimeric oncolytic virus of the present disclosure comprises one or more transgenes, for example one or more transgenes encoding therapeutic peptide(s) or protein sequence(s).

In one embodiment the chimeric oncolytic virus encodes at least one transgene. Suitable transgenes include so called suicide genes such as p53; polynucleotide sequences encoding cytokines such as IL-2, IL-6, IL-7, IL-12, IL-15, IL-18, IL-21, GM-CSF or G-CSF, an interferon (e.g. type I interferon such as IFN-alpha or beta, type II interferon such as IFN-gamma), a TNF (e.g. TNF-alpha or TNF-beta), TGF-beta, CD22, CD27, CD30, CD40, CD120; a polynucleotide encoding a monoclonal antibody, for example trastuzamab, cetuximab, panitumumab, pertuzumab, epratuzumab, an anti-EGF antibody, an anti-VEGF antibody and anti-PDGF antibody, an anti-FGF antibody, checkpoint inhibitor antibodies including anti-CTLA4, anti-PD1 and anti-PDL1 antibodies, or target antigen-binding fragments thereof, or tumour associated antigens such as NY-ESO1, WT1, MAGE-A3 and others known in the art.

A range of different types of transgenes, and combinations thereof, are envisaged that encode molecules that themselves act to modulate tumour or immune responses and act therapeutically, or are agents that directly or indirectly inhibit, activate or enhance the activity of such molecules. Such molecules include protein ligands or active binding fragments of ligands, antibodies (full length or fragments, such as Fv, ScFv, Fab, F(ab)′2 or smaller specific binding fragments), or other target-specific binding proteins or peptides (e.g. as may be selected by techniques such as phage display etc), natural or synthetic binding receptors, ligands or fragments, specific molecules regulating the transcription or translation of genes encoding the targets (e.g. siRNA or shRNA molecules, transcription factors). Molecules may be in the form of fusion proteins with other peptide sequences to enhance their activity, stability, specificity etc (e.g. ligands may be fused with immunoglobulin Fc regions to form dimers and enhance stability, fused to antibodies or antibody fragments having specificity to antigen presenting cells such as dendritic cells (e.g. anti-DEC-205, anti-mannose receptor, anti-dectin). Transgenes may also encode reporter genes that can be used, for example, for detection of cells infected with the “insert-bearing adenovirus”, imaging of tumours or draining lymphatics and lymph nodes etc.

In one embodiment the cancer cell infected with the chimeric oncolytic virus is lysed releasing the contents of the cell which may include the protein encoded by a transgene.

In one embodiment 40 to 93% or more of the total virus replicated in the cells is recoverable from the media, for example 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 or 92% of the total virus is recoverable, such as 94, 95, 96, 97, 98, 99 or 100% of the total virus recoverable.

In one embodiment the process is a GMP manufacturing process, such as a cGMP manufacturing process.

In one embodiment the process further comprises the step formulating the virus in a buffer suitable for storage.

In one embodiment the formation is suitable for storage at room temperature.

In one embodiment the formation is suitable for storage at 4° C. or below.

In one embodiment the formation is frozen.

In one embodiment the present disclosure extends to virus or viral formulations obtained or obtainable from the present method.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined, even if they relate to different independent aspects of the disclosure because the basic technology underlying all the inventions herein is the same.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present invention is further described by way of illustration only in the following examples, which refer to the accompanying Figures.

### Paragraphs

1. A continuous process for the manufacture of:
   a replication competent adenovirus; or
   a replication capable or deficient chimeric oncolytic adenovirus having a genome comprising an E2B region, wherein said E2B region comprises a nucleic acid sequence derived from a first adenoviral serotype and a nucleic acid sequence derived from a second distinct adenoviral serotype; wherein said first and second serotypes are each selected from the adenoviral subgroups B, C, D, E, or F, wherein the process comprises the steps:
   A) continuously culturing, in a vessel, mammalian cells infected with the adenovirus in the presence of media suitable for supporting the cells such that the virus replicates, wherein the cells are capable of supporting viral replication, and
   B) isolating from the media the virus produced from step a) wherein the isolation of virus is not subsequent to a cell lysis step,
   wherein viable cells for virus infection and production are maintained in the vessel at a level suitable for replicating the virus for the period of continuous manufacture.
   OR
1. A continuous process for the manufacture of:
   a type B adenovirus wherein the process comprises the steps:
   C) continuously culturing, in a vessel, mammalian cells infected with the adenovirus in the presence of media suitable for supporting the cells such that the virus replicates, wherein the cells are capable of supporting viral replication, and
   D) isolating from the media the virus produced from step a) wherein the isolation of virus is not subsequent to a cell lysis step,
   wherein viable cells for virus infection and production are maintained in the vessel at a level suitable for replicating the virus for the period of continuous manufacture.
2. A process according to paragraph 1, wherein the virus has a hexon and fibre from a group B adenovirus, for example Ad11 and in particular wherein the virus is selected from the group ColoAd1.
3. A process according to paragraph 1 or 2, wherein the virus is replication competent.
4. A process according to any one of paragraph 1 to 3, wherein the continuous manufacturing period comprises at least two virus replication cycles.
5. A process according to paragraph 4, wherein each virus replication cycle is in the range 70 to 300 hours.
6. A process according to any one of paragraph 1 to 4, wherein viable cells for virus infection and production are maintained in the vessel at a level to suitable for replicating the virus by the addition further cells to augment the culture.
7. A process according to any one of paragraph 1 to 6, wherein the mammalian cells are selected from the group comprising HEK, CHO, HeLa, Vero, PerC6 and GMK, in particular HEK293.
8. A process according to any one of paragraph 1 to 7, wherein the culture is a scale of 5 L or more.
9. A process according to any one of paragraph 1 to 8, wherein virus during culture is at concentration in the range 40 to 150 ppc, such as 50 to 100 ppc.
10. A process according to any one of paragraph 1 to 9, wherein the cells are infected with a starting concentration of virus of 1-9×10⁴ vp/ml or greater, such as 1-9×10⁵, 1-9×10⁶, 1-9×10⁷, 1-9×10⁸, 1-9×10⁹, in particular 4 to 5×10⁶ vp/ml.
11. A process according to any one of paragraph 1 to 10, which provides a fraction of oncolytic virus wherein the process comprises a further step such that a second fraction or fractions of the oncolytic virus made by the same of a different process is/are combined with the first fraction.
12. A process according to any one of paragraph 1 to 11, wherein a perfusion culture is employed.
13. A process according to any one of paragraph 1 to 12, wherein a suspension culture is employed.
14. A process according to any one of paragraph 1 to 12, wherein an adhesion culture is employed.
15 A process according to any one of paragraph 1 to 14, wherein the process is a GMP manufacturing process.
16. A process according to any one of paragraph 1 to 15, wherein the filter is a tangential filter.
17. A process according to any one of paragraph 1 to 16, wherein the process further comprises a purification step, selected from a CsCl gradient, chromatography step such as ion-exchange chromatography in particular anion-exchange chromatography, and a combination thereof.
18. A process according to any one of paragraph 1 to 17, wherein 40 to 93% of the total virus is recoverable from the media.
19. A process for the manufacture of an adenovirus comprises the steps:
   a. culturing, in a vessel, mammalian cells infected with the adenovirus in the presence of media suitable for supporting the cells such that the virus replicates, wherein the cells are capable of supporting viral replication, wherein the starting seed density of the virus is in the range 1 to 2×10⁶ vp/ml (such as 1×10⁶ vp/ml) and the multiplicity of infection is in the range 5 to 20, such as 10 to 15, in particular 12.5 vp/cell; and
   b. performing a lysis step in the period 24 to 75 hours post virus infection to harvest the virus from the cells, for example where the lysis step is performed at 65 to 70 hours post infection, such as 66, 67, 68 or 69 hours post infection.
20. A process according to any one of paragraph 1 to 19, which further comprises formulating the virus in a buffer suitable for storage.
21. A virus or formulation obtained or obtainable from this process described herein, for example in any one of claims 1 to 20.

### Sequences

SEQ ID NO: 1 NG-77 virus genome sequence comprising the EnAd genome with a transgene cassette that encodes an anti-VEGF full length antibody inserted in the region B_{Y}. The transgene cassette contains a 5′ branched splice acceptor sequence (bSA), ab heavy chain sequence with 5′ leader, an IRES, an ab light chain sequence with 5′ leader and a 3′ poly(A) sequence.
SEQ ID NO: 2 NG-135 virus genome sequence comprising the EnAd genome with a transgene cassette that encodes an anti-VEGF full length antibody inserted in the region B_{Y}. The transgene cassette contains a 5′ short splice acceptor sequence (SSA), ab heavy chain sequence with 5′ leader, an IRES, ab light chain sequence with 5′ leader and 3′ poly(A) sequence.
SEQ ID NO: 3 A virus genome sequence comprising a transgene cassette that encodes an anti-VEGF full length antibody inserted in the region B_{Y}. The transgene cassette contains a SSA, ab heavy chain sequence with 5′ leader, a SSA, and ab light chain sequence with 5′ leader.
SEQ ID NO: 4 A virus genome sequence comprising a transgene cassette that encodes an anti-VEGF full length antibody inserted in the region B_{Y}. The transgene cassette contains a SSA, ab heavy chain sequence with 5′ leader, a SSA, ab light chain sequence with 5′ leader and 3′ poly(A) sequence.
SEQ ID NO: 5 NG-74 virus genome sequence comprising the EnAd genome with a transgene cassette that encodes an anti-VEGF ScFv inserted in the region B_{Y}. The transgene cassette contains a bSA, anti-VEGF ScFv sequence with 5′ leader and 3′ poly(A) sequence.
SEQ ID NO: 6 NG-78 virus genome sequence comprising the EnAd genome with a transgene cassette that encodes an anti-VEGF ScFv with a C-terminal His₆ tag, inserted in the region B_{Y}. The transgene cassette contains a bSA, anti-VEGF ScFv sequence with 5′ leader and 3′ 6×histidine sequence and a poly(A) sequence.
SEQ ID NO: 7 NG-76 virus genome sequence comprising the EnAd genome with a transgene cassette that encodes an anti-VEGF ScFv with a C-terminal His₆ tag, inserted in the region B_{Y}. The transgene cassette contains a CMV promoter, anti-VEGF ScFv sequence with 5′ leader and 3′ 6×histidine sequence and a poly(A) sequence.
SEQ ID NO: 8 NG-73 virus genome sequence comprising the EnAd genome with a transgene cassette that encodes an anti-VEGF ScFv inserted in the region B_{Y}. The transgene cassette contains a CMV promoter, anti-VEGF ScFv sequence with 5′ leader and 3′ poly(A) sequence.
SEQ ID NO: 9 NG-134 virus genome sequence comprising the EnAd genome with a transgene cassette encoding an anti-VEGF full length antibody inserted into the region B_{Y}. The transgene cassette contains a CMV promoter, ab heavy chain sequence with 5′ leader, an IRES, ab light chain sequence with 5′ leader and a 3′ poly(A) sequence.
SEQ ID NO: 10 B_{X} DNA sequence corresponding to and including bp 28166-28366 of the EnAd genome.
SEQ ID NO: 11 B_{Y} DNA sequence corresponding to and including bp 29345-29379 of the EnAd genome.
SEQ ID NO: 12 EnAd genome.
SEQ ID NO: 13 CMV exogenous promoter.
SEQ ID NO: 14 PGK exogenous promoter.
SEQ ID NO: 15 CBA exogenous promoter.
SEQ ID NO: 16 Short splice acceptor (SSA). Null sequence
SEQ ID NO: 17 splice acceptor (SA).
SEQ ID NO: 18 branched splice acceptor (bSA).
SEQ ID NO: 19 Internal Ribosome Entry sequence (IRES).
SEQ ID NO: 20 polyadenylation sequence.
SEQ ID NO: 21 Leader sequence (HuVH).
SEQ ID NO: 22 Leader sequence (HG3).
SEQ ID NO: 23 Histidine tag.
SEQ ID NO: 24 V5 tag.
SEQ ID NO: 25 P2A peptide.
SEQ ID NO: 26 F2A peptide.
SEQ ID NO: 27 E2A peptide.
SEQ ID NO: 28 T2A peptide.
SEQ ID NO: 29 anti-VEGF ab VH chain amino acid sequence.
SEQ ID NO: 30 anti-PD-L1 antibody VH chain amino acid sequence.
SEQ ID NO: 31 anti-VEGF ab VL chain amino acid sequence.
SEQ ID NO: 32 anti-PD-L1 antibody VL chain amino acid sequence.
SEQ ID NO: 33 human IgG1 constant heavy chain amino acid sequence.
SEQ ID NO: 34 human IgG1 modified constant heavy chain amino acid sequence.
SEQ ID NO: 35 human kappa constant light chain amino acid sequence.
SEQ ID NO: 36 anti-VEGF ScFv amino acid sequence.
SEQ ID NO: 37 anti-PD-L1 ScFv amino acid sequence.
SEQ ID NO: 38 Green fluorescent protein amino acid sequence.
SEQ ID NO: 39 Luciferase amino acid sequence.
SEQ ID NO: 40 Human Tumour necrosis factor alpha (TNFα) amino acid sequence.
SEQ ID NO: 41 Human Interferon gamma (IFNγ) amino acid sequence.
SEQ ID NO: 42 Human Interferon alpha (IFNα) amino acid sequence.
SEQ ID NO: 43 human cancer/testis antigen 1 (NY-ESO-1) amino acid sequence.
SEQ ID NO: 44 human MUC-1 amino acid sequence.
SEQ ID NO: 45 A Kozak sequence. gccaccatg (Null sequence)
SEQ ID NO: 46 NG-177 virus genome sequence comprising the EnAd genome with a transgene cassette. encoding an anti-PD-L1 full length antibody inserted into the region B_{Y}. The transgene cassette contains a CMV promoter, ab heavy chain sequence with 5′ leader, an IRES, ab light chain sequence with 5′ leader and a 3′ poly(A) sequence.
SEQ ID NO: 47 DNA sequence corresponding to E2B region of the EnAd genome (bp 10355-5068).
SEQ ID NO: 48 NG-167 virus genome sequence comprising the EnAd genome with a transgene cassette that encodes an anti-VEGF ScFv with a C-terminal His₆ tag, inserted in the region B_{Y}. The transgene cassette contains a 5′ SSA, anti-VEGF ScFv sequence with 5′ leader and a 3′ poly(A) sequence.
SEQ ID NO: 49 NG-95 virus genome sequence comprising a transgene cassette that encodes the cytokine, IFNγ, inserted in the region B_{Y}. The transgene cassette contains a 5′ CMV promoter, IFNγ cDNA sequence and 3′ poly(A) sequence.
SEQ ID NO: 50 NG-97 virus genome sequence comprising a transgene cassette that encodes the cytokine, IFNα, inserted in the region B_{Y}. The transgene cassette contains a 5′ CMV promoter, IFNα cDNA sequence and 3′ poly(A) sequence.
SEQ ID NO: 51 NG-92 virus genome sequence comprising the EnAd genome with a transgene cassette that encodes the cytokine, IFNγ, inserted in the region B_{Y}. The transgene cassette contains a 5′ bSA, IFNγ cDNA sequence and 3′ poly(A) sequence.
SEQ ID NO: 52 NG-96 virus genome sequence comprising the EnAd genome with a transgene cassette that encodes the cytokine, IFNα, inserted in the region B_{Y}. The transgene cassette contains a 5′ bSA, IFNα cDNA sequence and 3′ poly(A) sequence.
SEQ ID NO: 53 NG-139 virus genome sequence comprising the EnAd genome with a transgene cassette that encodes the cytokine, TNFα, inserted in the region B_{Y}. The transgene cassette contains a 5′ SSA, TNFα cDNA sequence and 3′ poly(A) sequence.
SEQ ID NO: 54 Restriction site insert (B_{Y}).
SEQ ID NO: 55 Restriction site insert (B_{Y}).
SEQ ID NO: 56 NG-220 virus genome sequence comprising the EnAd genome with a transgene cassette that encodes the tumour associated antigen, NY-ESO-1, inserted in the region B_{Y}. The transgene cassette contains a 5′ PGK promoter, NY-ESO-1 cDNA sequence and 3′ poly(A) sequence.
SEQ ID NO: 57 NG-217 virus genome sequence comprising the EnAd genome with a transgene cassette that encodes the tumour associated antigen, NY-ESO-1, inserted in the region B_{Y}. The transgene cassette contains a 5′ CMV promoter, NY-ESO-1 cDNA sequence and 3′ poly(A) sequence.
SEQ ID NO: 58 NG-242 virus genome sequence comprising the EnAd genome with a transgene cassette encoding an anti-CTLA-4 full length antibody inserted into the region B_{Y}. The transgene cassette contains a SSA, ab heavy chain sequence with 5′ leader, an IRES, ab light chain sequence with 5′ leader and a 3′ poly(A) sequence.
SEQ ID NO: 59 NG-165 virus genome sequence comprising the EnAd genome with a transgene cassette encoding an anti-VEGF full length antibody inserted into the region B_{Y}. The transgene cassette contains a SSA, ab heavy chain sequence with 5′ leader, a P2A peptide sequence, ab light chain sequence with 5′ leader and a 3′ poly(A) sequence.
SEQ ID NO: 60 NG-190 virus genome sequence comprising the EnAd genome with a transgene cassette encoding an anti-PD-L1 full length antibody inserted into the region B_{Y}. The transgene cassette contains a SSA, ab heavy chain sequence with 5′ leader, a P2A peptide sequence, ab light chain sequence with 5′ leader and a 3′ poly(A) sequence.
SEQ ID NO: 61 NG-221 virus genome sequence comprising the EnAd genome with a transgene cassette that encodes an anti-PD-L1 ScFv with a C-terminal His₆ tag, inserted in the region B_{Y}. The transgene cassette contains a 5′ SSA, anti-PD-L1 ScFv sequence with 5′ leader and 3′ 6×histidine sequence then poly(A) sequence.
SEQ ID NO: 62 NG-258 virus genome sequence comprising the EnAd genome with a transgene cassette encoding an anti-VEGF full length antibody inserted into the region B_{Y}. The transgene cassette contains a CMV promoter, ab heavy chain sequence with 5′ leader, a P2A peptide sequence, ab light chain sequence with 5′ leader and a 3′ poly(A) sequence.
SEQ ID NO: 63 NG-185 virus genome sequence comprising the EnAd genome with unique restriction sites inserted into the B_{X} and B_{Y} regions.
SEQ ID NO:64 pNG-33 (pColoAd2.4) DNA plasmid, comprising a bacterial origin of replication (p15A), an antibiotic resistance gene (KanR) and the EnAd genome sequence with inserted unique restriction sites in the B_{Y} region.
SEQ ID NO: 65 pNG-185 (pColoAd2.6) DNA plasmid, comprising a bacterial origin of replication (p15A), an antibiotic resistance gene (KanR) and the EnAd genome sequence with inserted unique restriction sites in the B_{X} and B_{Y} regions.
SEQ ID NO: 66 NG-sh01 virus genome sequence comprising a transgene cassette encoding an shRNA to GAPDH inserted into the region B_{Y}. The transgene cassette contains a U6 RNA polIII promoter and DNA encoding a shRNA.
SEQ ID NO: 67 Sodium Iodide symporter (NIS) amino acid sequence.
SEQ ID NO: 68 NG-280 virus genome sequence comprising a transgene cassette encoding the sodium iodide symporter (NIS) inserted into the region B_{Y}. The transgene cassette contains a 5′ SSA, NIS cDNA sequence and 3′ poly(A) sequence.
SEQ ID NO: 69 NG-272 virus genome sequence comprising the EnAd genome with a transgene cassette encoding an anti-VEGF ScFv and an anti-PD-L1 ScFv inserted into the region B_{Y}. The transgene cassette contains a SSA, anti-PD-L1 ScFv sequence with 5′ leader and 3′ 6×His tag, a P2A peptide sequence, anti-VEGF ScFv sequence with 5′ leader and 3′ V5-tag and a 3′ poly(A) sequence.
SEQ ID NO: 70 anti-CTLA-4 VH chain amino acid sequence.
SEQ ID NO: 71 anti-CTLA-4 VL chain amino acid sequence.
SEQ ID NO: 72 NG-257 virus genome sequence comprising the EnAd genome with a transgene cassette encoding an anti-VEGF ScFv inserted into the region B_{X}. The transgene cassette contains a bSA, anti-VEGF ScFv sequence with 5′ leader and 3′ 6×His tag then a 3′ poly(A) sequence.
SEQ ID NO: 73 NG-281 virus genome sequence comprising the EnAd genome with a transgene cassette encoding an anti-VEGF ScFv inserted into the region B_{X} and a second transgene cassette encoding an anti-PD-L1 ScFv inserted into the region B_{Y}. The transgene cassette contains a bSA, anti-VEGF ScFv sequence with 5′ leader and 3′ 6×His tag then a 3′ poly(A) sequence.

### BRIEF DESCRIPTION OF THE FIGURES

FIG. 1 shows a schematic representation of adenovirus production process

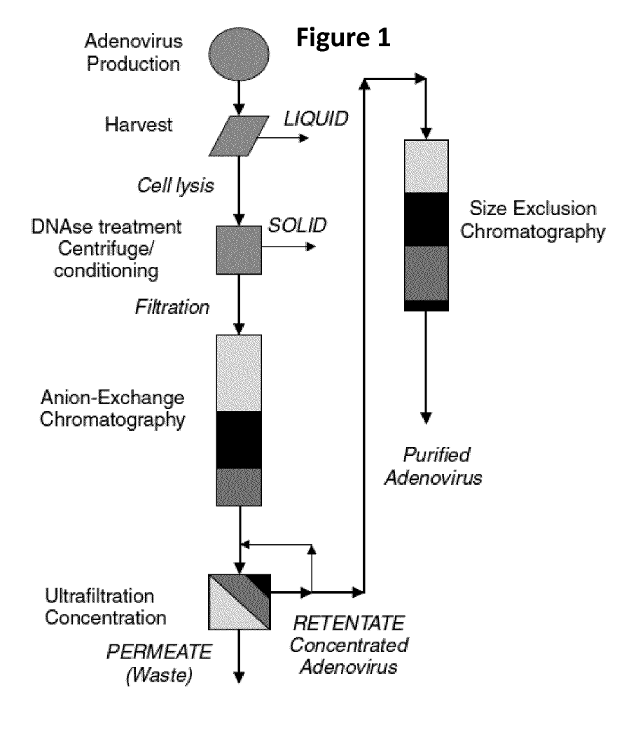

FIG. 2 shows a cell culturing arrangement for continuous virus manufacture

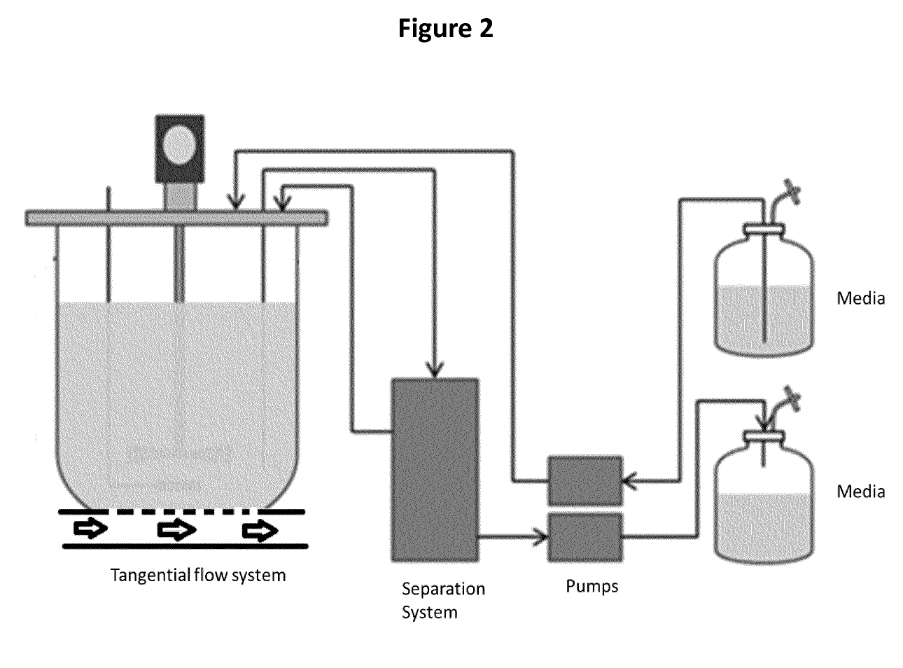

FIG. 3 shows a plot of seed density vs yield

FIG. 4 shows a plot of virus produced per cell at various time points post infection for shake flasks A & B with a Multiplicity of Infection of 12.5 and seed cell density of 1×10⁶ and media change

FIG. 5 shows a plot of virus produced per cell at various time points post infection for shake flasks C & D with a Multiplicity of Infection of 12.5 and seed cell density of 4×10⁶ and NO media change

FIG. 6 shows that media changes increase virus yield

FIG. 7 shows percentage viabilty of cells vs duration of infection

FIG. 8 shows percentage of cell viability at various times post infection for DOE (??) Fflask A & B with a multiplicity of infection of 12.5 and seed density of 1×10⁶ with media change

FIG. 9 shows percentage of virus in the supernatant for various time points post infection for DOE flasks A & B with a multiplicity of infection of 12.5 and a seed density of 1×10⁶ with media change

FIG. 10 shows percentage of virus in the supernatant for various time points post infection for DOE flasks C & D with a multiplicity of infection of 12.5 and a seed density of 4×10⁶ with no media change

FIG. 11 shows the virus product distribution of a 5 L bioreactor process with cells infected with EnAd at an MOI of 12.5 ppc and a cell density of 1×10⁶ cell/ml.

FIG. 12 shows the total virus particles produced per cell over time for a process employing a seed density of 1.9×10⁶ cells/ml and the culture was infected with EnAd at 50 ppc

FIG. 13 shows cell viability over time for a process employing 12.5 ppc and a seed density of 1×10⁶ vp/ml

FIG. 14 shows cell viability over time for a process employing 50 ppc and a seed density of 1.9×10⁶ vp/ml

FIG. 15 shows bioreactor data for two process one employing a seed density of 1×10⁶ vp/ml and 12.5 ppc and a second process employing 1.9×10⁶ vp/ml and 50 ppc.

FIG. 16 shows cell viability over time for a process employing a seed density of 2.2×10⁶ viable cells/mL and 50 ppc infection

FIG. 17 shows the cell distribution for a process employing a seed density of 2.2×10⁶ viable cells/mL and 50 ppc infection

FIG. 18 shows viability of control cultures employing a seed density of 2.2×10⁶ viable cells/mL and infected with 50 ppc but no cell suspension removal or addition of fresh cells and medium.

FIG. 19 shows virus distribution for control cultures employing a seed density of 2.2×10⁶ viable cells/mL and infected with 50 ppc but no cell suspension removal or addition of fresh cells and medium.

FIG. 20 shows cumulative virus distribution for a process employing a seed density of 2.2×10⁶ viable cells/mL and 50 ppc infection and a control process omitting no cell suspension removal and no addition of fresh cells and medium.

FIG. 21 shows cell viability over time for a process employing 1.0×10⁶ viable cells/mL and 12.5 ppc infection in a shake flask experiment.

FIG. 22 shows virus distribution over time for a process employing 1.0×10⁶ viable cells/mL and 12.5 ppc infection in a shake flask experiment.

FIG. 23 shows cell viability over time for a control process employing 1.0×10⁶ viable cells/mL and 12.5 ppc infection in a shake flask experiment where omitted was cell suspension removal and no addition of fresh cells and medium.

FIG. 24 shows virus distribution over time for a control process employing 1.0×10⁶ viable cells/mL and 12.5 ppc infection in a shake flask experiment where omitted was cell suspension removal and no addition of fresh cells and medium.

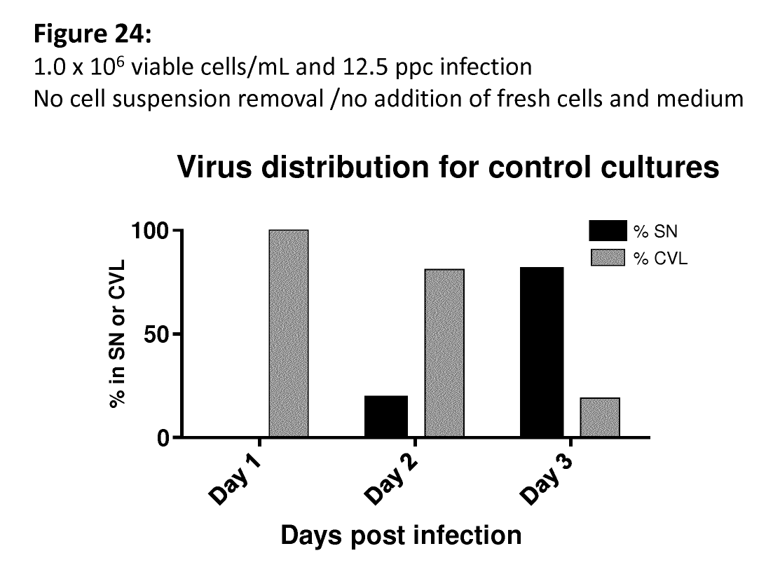

FIG. 25 shows cumulative total virus yields for a process employing 1.0×10⁶ viable cells/mL and 12.5 ppc infection in a shake flask experiment and a control process where omitted was cell suspension removal and no addition of fresh cells and medium.

FIG. 26 shows viability of cells in a process with 1.0×10⁶ viable cells/mL and 12.5 ppc infection wherein 95% suspension was removed at various time points & replaced with 47.5 mL uninfected cells in fresh medium cell density of 1.0×10⁶ viable cells/ml.

FIG. 27 shows virus distribution in a process with 1.0×10⁶ viable cells/mL and 12.5 ppc infection wherein 95% suspension was removed at various time points & replaced with 47.5 mL uninfected cells in fresh medium cell density of 1.0×10⁶ viable cells/ml.

FIG. 28 shows cumulative total virus in a process with 1.0×10⁶ viable cells/mL and 12.5 ppc infection wherein 95% suspension was removed at various time points & replaced with 47.5 mL uninfected cells in fresh medium cell density of 1.0×10⁶ viable cells/ml and a control process.

### EXAMPLES

### Example 1

Suspension culture and serum-free media adapted HEK293 cells are grown and expanded to a cell density of 1×10⁶ viable cells/mL at >90% viability in 30 mL EX-CELL serum-free medium in multiple 125 mL shake-flasks at 100 rpm. Some of the flasks are then infected with EnAd virus using a multiplicity of infection (MOI) of 50 virus particles per cell (ppc), while the remainder are left uninfected and are maintained at a cell density between 0.5 and 2×10⁶ viable cells/mL and viability >75% by passaging and used for adding back to the infected cell cultures as indicated below. At various time points post infection, 10 mL of the culture is removed from one of the shake flasks and replaced with 10 mL of uninfected HEK293 cells in fresh EX-CELL medium at 1×10⁶ viable cells/mL and >90% viability. The 10 ml of infected cell culture suspension is centrifuged, the supernatant is stored for analysis and the cell pellet is lysed in 1 mL fresh medium (3× freeze-thaw) which is then clarified by centrifugation prior to storing for analysis. The infected shake flask cultures are then re-cultured until the cell viability decreased to <75% and then cultures are terminated, centrifuging and processed to generate supernatant and cell lysate fractions (as above) which are stored for analysis. Throughout the experiment, small aliquots of suspension (50 μl) are removed from each flask for cell and viability counts using a Burker cell hemacytometer and trypan blue staining, as well as pH measurements.

Total and infectious EnAd particles in cell lysate and supernatant samples are determined by HPLC and immunostaining infection assays, respectively. The amount of host cell DNA in supernatant samples is determined by real time qPCR and the amount of host cell protein in supernatants is determined using a HEK293 Host Cell Protein ELISA kit using affinity purified goat anti-HEK antiserum. Total EnAd virus yields and levels of host cell DNA and protein were compared for the different processing time points.

### Example 2

An experimental protocol is employed as described in Example 1 but with 15 mL of the 30 mL suspension being removed at the various time points and replaced with 15 mL uninfected cells in fresh medium instead of 10 mL volumes.

### Example 3

An experimental protocol is employed as described in Example 1 but in this experiment the fresh uninfected cells added back to the infected culture flasks are resuspended in the virus-containing supernatant removed from that same flask (keeping back 2×500 μl aliquots for analysis) and the volume adjusted to 10 mL prior to adding back to the original flask.

### Example 4

An experimental protocol is employed as described in Example 3 but with 15 mL of the 30 mL suspension being removed at the different time points and replaced with uninfected cells resuspended in 15 mL of the virus-containing supernatant instead of 10 mL volumes.

### Example 5 to 8

A set of four further experiments employing the protocols detailed in examples 1-4, but rather than terminating the experiment after one round of suspension removal and replacement with fresh cells, the cultures are maintained on a repeating suspension removal and fresh cell replacement protocol which is continued until maintenance of sufficient viable cells could not be sustained (e.g. due to build-up of cell debris or other cellular products or lack of sufficient nutrients). The frequency of suspension removals and replacements were guided by daily cell counting and viability information together with visual observations.

### Example 9

A single vial of suspension HEK293 cells are thawed and expanded in shake flasks prior to expansion to a 3 L working volume in a 5 L stirred-tank (glass vessel) bioreactor. The bioreactor controller is set to parameters of 37° C., a pH setpoint of 7.4, dissolved oxygen (DO) of 50, an airflow rate of 100 mL/min, and the agitation at 100 rpm. After the bioreactor system is equilibrated, an initial volume of 1.5 L EX-CELL culture medium is seeded at a viable cell density of 5×10⁵ cells/mL and then expanded to a working volume of 3 L maintaining >90% viability and targeting a density of 1.8 to 2.2×10⁶ cells/mL for infection with EnAd virus. Perfusion and media exchange is initiated at the 3 L stage via hollow fibre tangential flow filtration (TFF). The TFF cartridge had a 0.2 um pore size, 0.5 mm lumen diameter, and a surface area of 790 cm². The TFF assembly was pre-sterilized by autoclaving, and then attached to the bioreactor through the use of a sterile tubing welder. Using a peristaltic pump, the cell culture is recirculated through the TFF system. No backpressure was placed onto the retentate line, while a second peristaltic pump is placed onto the permeate line to provide a measured flow rate out of the system. The perfusion flow rate was set to 1 vessel volume per day (i.e. 3 L/24 h). Once the target cell density was reached, the culture was infected with EnAd at an MOI of 50 ppc.

In parallel with the bioreactor culture, shake flask cultures of HEK293 cells are established and maintained at a cell density of 0.5-2×10⁶ cells/mL and >90% viability by regular passaging and these cells are used as source of uninfected cells to add into the bioreactor as described.

Collection of the perfusion permeate was started 48 hours post infection and perfusion permeate samples are taken at regular time points, with non-infected cells from the second suspension flasks added back to the infected cells in the bioreactor, maintaining the cell density at 2×10⁶ cells/mL and cell viability >75% viability. Total and infectious EnAd particles in cell permeate samples were determined by HPLC and immunostaining infection assays, respectively. The amount of host cell DNA in supernatant samples was determined by real time qPCR and the amount of host cell protein in was determined using a HEK293 Host Cell Protein ELISA kit using affinity purified goat anti-HEK antiserum. Total EnAd virus yields and levels of host cell DNA and protein at various time points are compared. The bioreactor culture is actively maintained by the perfusion and fresh uninfected cell replacement procedure until the cell viability could not be maintained above 50%.

Collected virus-containing permeate samples are pooled and virus purified by a process previously established for GMP manufacture of EnAd virus (outlined below) such that analytical assay data could be compared to appropriate standard virus preparations that had been manufactured without the elements of continuous manufacturing described herein.

### EnAd Virus Purification

Virus is purified from the cell-free permeate samples collected from the bioreactor at different time points. These permeate samples are first treated with Benzonase® to digest host cell DNA and then concentrated and buffer exchanged by tangential flow filtration (TFF) in preparation for the first of a two-step purification process in the downstream which involves the selective capture and elution of EnAd using two different anion exchange chromatography resins. The first primary capture step (e.g. Sartobind resin) is followed by a second “polishing” purification step (e.g. CIM-Q) to reduce host cell residuals further. The purified virus is then buffer exchanged into the final formulation buffer using a second TFF step prior to the material being stored frozen at −80° C. prior to analyses.

### Example 10

An experimental protocol as described in example 9 is employed with an MOI of 100 used for the EnAd infection of the HEK293 cells.

### Example 11

For this experiment, a Design of Experiment (DOE) approach was followed and customised using JMP software in order to evaluate effects of different culture parameters on virus yields and distribution into supernatant or cells. The different variables, range of each variable and responses used for the design of this study are shown in Table 1.

TABLE 1
Variables and responses for design of experiment

| Factors/Variables | Range | Responses |
|---|---|---|
| Multiplicity of | 12.5-50 ppc | Yield: Virus particles/cell |
| Infection (MOI) | | |
| Seeding Density | 1 × 10⁶-4 × 10⁶ cells/ml | Virus distribution: % SN, |
| | | % CVL |
| Media change | Yes/No | % Viability |
| Duration of | 40-96 hrs | Host cell proteins |
| Infection (DOI) | | |

One vial of HEK293 cells from a working cell bank (WCB) was thawed at 37° C. and expanded in 75 cm² cell culture flasks using Ex-Cell 293 medium supplemented with 6 mM L-glutamine (growth medium). After 4 days of incubation the cells were further passaged (Passage 1) and once a viable cell count of 1.2×10⁷ cells at a density of >1.0×10⁶ viable cells/mL was achieved (approximately 3-5 days after passage 1), the cells were transferred into sterile shaker flasks (Passage 2). The cultures were monitored, and when the cell number had doubled to ≧1.2×10⁶ viable cells/mL, the cells were further passaged approximately every 3 or 4 days. Cell numbers were monitored throughout the cell expansion phase by counting to ensure cell density was maintained at a minimum cell density of 0.5-0.6×10⁶ viable cells/mL.

Cell counting was performed using automatic cell counter (Invitrogen) and Trypan Blue staining (Invitrogen). Cell numbers were expanded further in 1 L sterile shaker flasks until the required cells for the experiment were generated.

The cell suspension was centrifuged at 300 g for 5 minutes and the cell pellet resuspended in fresh growth medium and the cell suspension transferred to shaker flasks at a working volume of 40 ml in each flask with the seeding cell density adjusted according to Table-2. Each shaker flask was labelled appropriately.

TABLE 2
Experiment design set up

| | | | | | | Duration |
|---|---|---|---|---|---|---|
| | | | | | | of |
| Shaker | | | | | | Infection |
| flask | MOI | Seed Cell | | Total | Media | [DOI] |
| ID | (ppc) | Density | Volume | Cells | Change | (hrs) |
| A | 12.5 | 1.00E+06 | 40 | 4.00E+07 | Yes | 40, 48, 60, |
| | | | | | | 72, 96 |
| B | 12.5 | 1.00E+06 | 40 | 4.00E+07 | Yes | 40, 48, 60, |
| | | | | | | 72, 96 |
| C | 12.5 | 4.00E+06 | 40 | 1.60E+08 | No | 40, 48, 60, |
| | | | | | | 72, 96 |
| D | 12.5 | 4.00E+06 | 40 | 1.60E+08 | No | 40, 48, 60, |
| | | | | | | 72, 96 |
| E | 31.25 | 2.50E+06 | 40 | 1.00E+08 | No | 40, 48, 60 |
| | | | | | | 72, 96 |
| F | 31.25 | 2.50E+06 | 40 | 1.00E+08 | No | 40, 48, 60, |
| | | | | | | 72, 96 |
| G | 31.25 | 2.50E+06 | 40 | 1.00E+08 | Yes | 40, 48, 60, |
| | | | | | | 72, 96 |
| H | 50 | 1.00E+06 | 40 | 4.00E+07 | No | 40, 48, 60, |
| | | | | | | 72, 96 |
| I | 50 | 4.00E+06 | 40 | 1.60E+08 | Yes | 40, 48, 60, |
| | | | | | | 72, 96 |
| Negative | 0 | 1.00E+06 | 40 | 4.00E+07 | NA | NA |
| control | | | | | | |

Note:
1.00E+06, 1e⁶ and 1 × 10⁶; 4.00E+07, 4e⁷ and 4 × 10⁷ etc are equivalent cell number descriptors

One vial of EnAd working virus seed stock (WVSS) was removed from −70° C. storage and thawed at room temperature. Infection of the shaker flasks A to I was performed using a multiplicity of infection (MOI or ppc) according to Table-2. A negative control flask was not infected but maintained throughout the duration of infection. All shaker flasks were placed in a shaking incubator at +37° C., 5% CO₂ and 120 rpm. Media change was performed on shake flasks A, B, G and I at 24 hrs post infection by removing the supernatant after centrifugation at 300 g for 5 min and resuspending the cell pellet in 40 ml fresh growth medium in each flask.

TABLE 3
Virus calculations for infections

| | | | | | | | Volume |
|---|---|---|---|---|---|---|---|
| Shaker | | | | | AEX titer | Total vp | of Virus |
| flask ID | Cells/ml | Volume | Total cells | ppc | of virus | needed | added (μl) |
| A | 1.00E+06 | 40 | 4.00E+07 | 12.5 | 1.35E+11 | 5E+08 | 3.7 |
| B | 1.00E+06 | 40 | 4.00E+07 | 12.5 | 1.35E+11 | 5E+08 | 3.7 |
| C | 4.00E+06 | 40 | 1.60E+08 | 12.5 | 1.35E+11 | 2E+09 | 14.8 |
| D | 4.00E+06 | 40 | 1.60E+08 | 12.5 | 1.35E+11 | 2E+09 | 14.8 |
| E | 2.50E+06 | 40 | 1.00E+08 | 31.25 | 1.35E+11 | 3.125E+09 | 23.1 |
| F | 2.50E+06 | 40 | 1.00E+08 | 31.25 | 1.35E+11 | 3.125E+09 | 23.1 |
| G | 2.50E+06 | 40 | 1.00E+08 | 31.25 | 1.35E+11 | 3.125E+09 | 23.1 |
| H | 1.00E+06 | 40 | 4.00E+07 | 50 | 1.35E+11 | 2E+09 | 14.8 |
| I | 4.00E+06 | 40 | 1.60E+08 | 50 | 1.35E+11 | 8E+09 | 59.2 |

At 40, 48, 60 and 72 hours post infection, 4.1 ml samples were taken from each flask for analyses and at 96 hours post-infection all the remaining cell suspensions were harvested. 2×500 μl of the samples at each time point were used for cell count and viability analysis. The remaining 4.0 ml was used for analysis of virus distribution between the supernatant and cell pellet. This was determined by centrifugation of the infected cell culture suspension and the supernatant stored for analysis and the cell pellet lysed in fresh medium (3× freeze-thaw) which was then clarified by centrifugation prior to storing for analysis.

Total viral particle concentrations (vp) in the Crude Viral Lysate (CVL) and supernatant (SN) samples were measured by AEX-HPLC assay. During AEX-HPLC analysis, it was known that host cell proteins (HCP) elute at the beginning of the elution run and thus HCP content can also be determined by analysis of the chromatogram and the size of the HCP peak area.

Cell percentage viability and percentage trypan blue stained cells (which represent both dead cells and “leaky” cells that are not yet functionally dead but have their membrane integrity compromised such that the trypan blue dye can enter the cell) is represented in Table 4. Total number of virus particles per shaker flask culture and the percentage of viral particles in the SN and CVL for each sample time point are represented in Table 5.

Results from this DOE experiment were fitted using the ‘least square fit model’ using JMP software analysis to assess the relationship between different variables and the effects of the variable on responses in relation to viral yield (vp/cell). Three interaction relationships were observed as significant in the yield per cell model, which were 1) seeding density to DOI; 2) MOI to DOI and 3) media change to DOI.

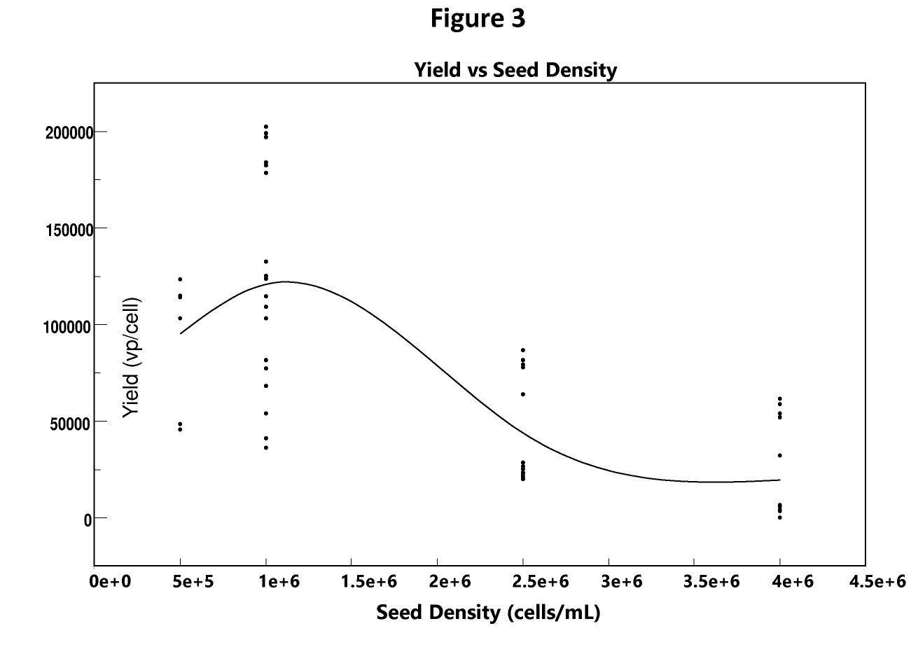

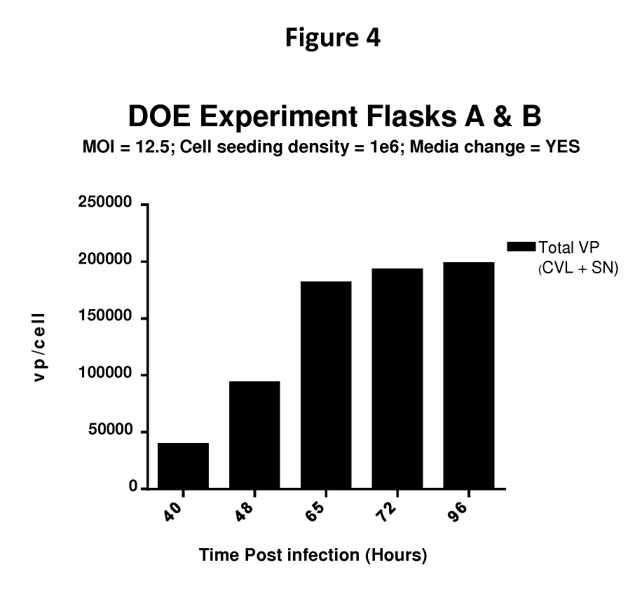

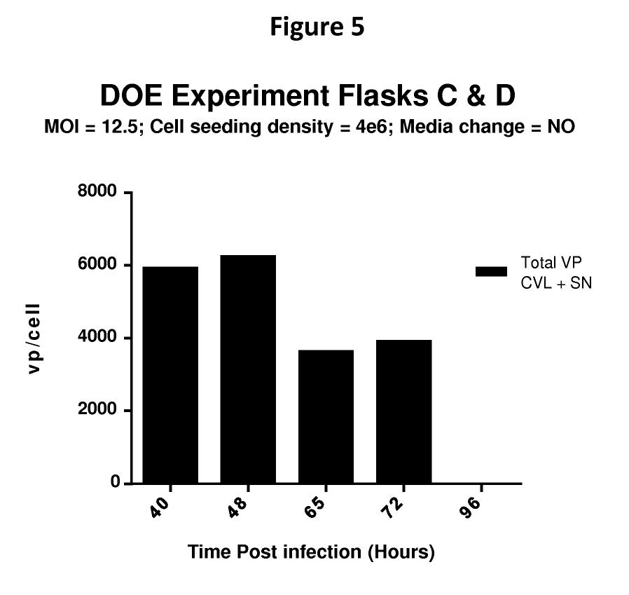

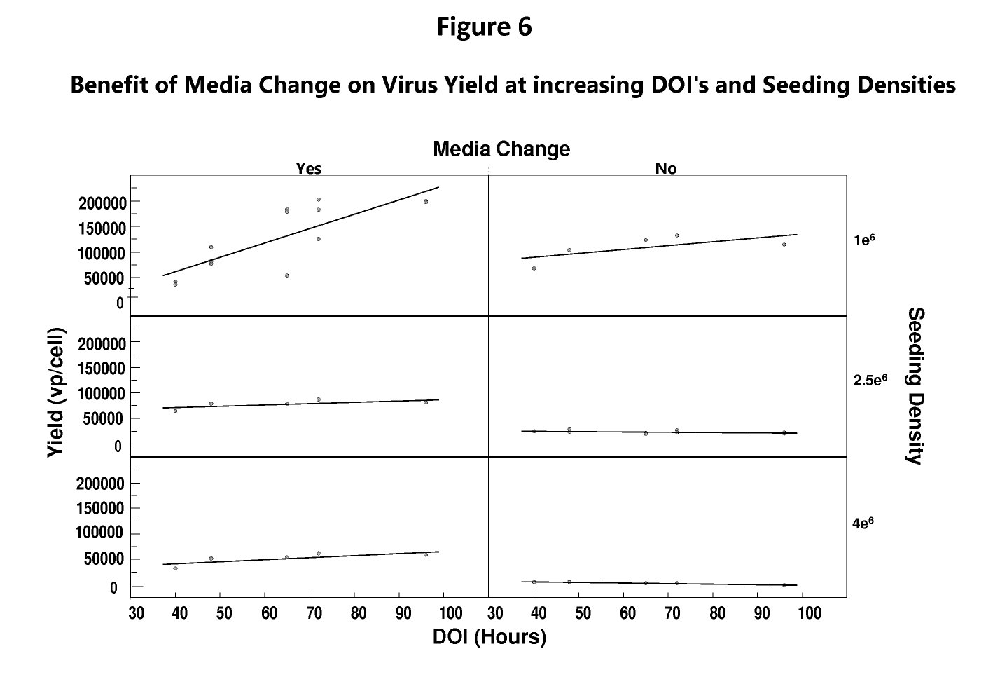

Seeding density had the largest statistical effect on total virus production. Higher yields per cell were observed at lower cell seeding density (FIG. 3). Highest yield of 198,143 vp/cell (FIGS. 3 & 4) was observed at the lowest cell density of 1e⁶ cells/ml (infected with 12.5 ppc) compared to 4e⁶ cells/ml (infected with 12.5 ppc) where the viral yield per cell was significantly less at 5922 vp/cell (FIGS. 3 & 5). Total virus yields per flask were highest at the higher cell density and MOI conditions (flask I). Media change also had a significant statistical effect on yield (FIG. 6). Shaker flasks which had media changes post 24 hrs infection (Shaker flask A, B, G and I) had higher virus yield compared to the shaker flask which had no media change. Results are shown in FIGS. 4, 5 and 6.

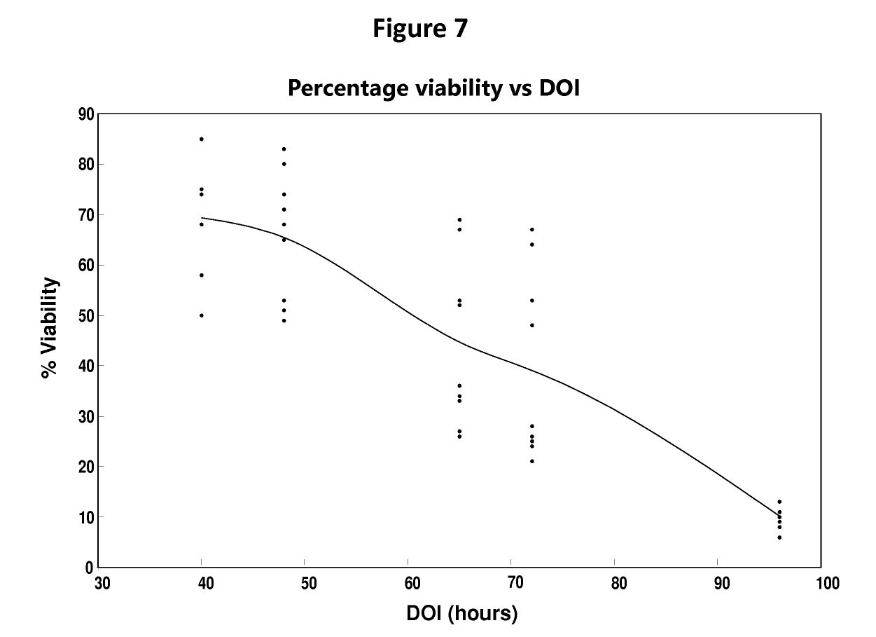

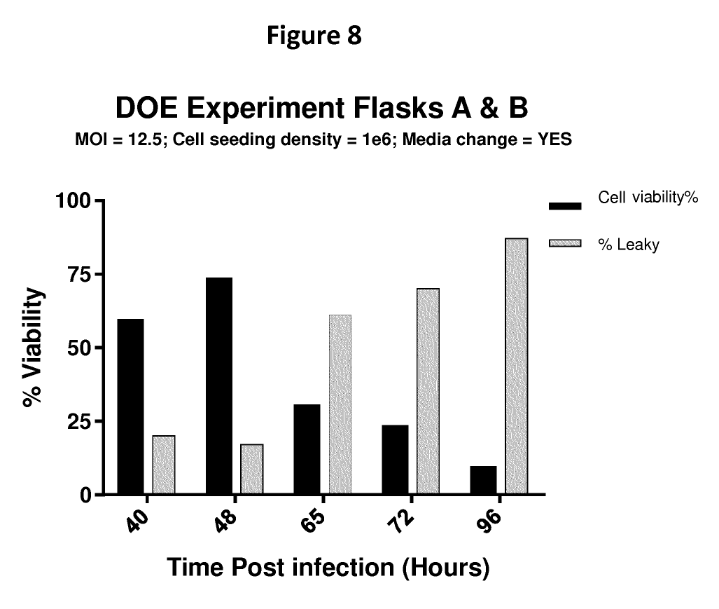

At higher duration of infection (DOI) cell viability decreased and the percentage of leaky and dead cells increased. Viability and % leaky/dead data is represented in Table 4 and shown in FIGS. 7 and 8. Leaky cells are defined as cells which appear to have an intact cell membrane but the membrane is permeable to trypan blue stain. Dead cells are trypan blue stained but the cell membrane is no longer intact.

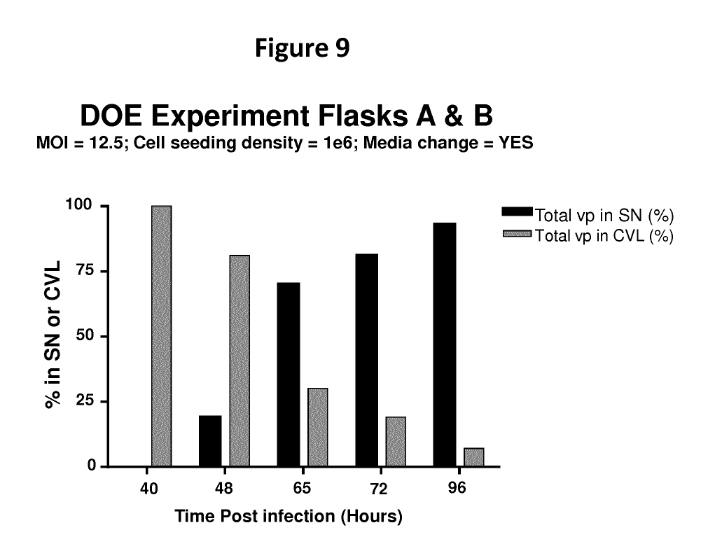

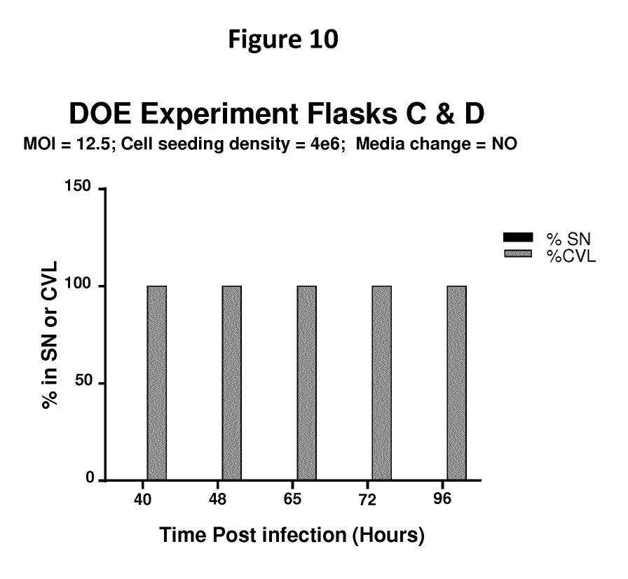

At 1e⁶ cells/ml seeding density infected with 12.5 ppc with a media change, more than 93% of virus was observed in supernatant at 96 hrs post infection (FIG. 9). At a higher cell density of 4e⁶ cells/ml, infected with the same 12.5 ppc with no media change, there was no virus observed in supernatant (FIG. 10).

TABLE 4
Viability data of shaker flasks

| time | | | | | | | |
|---|---|---|---|---|---|---|---|
| point | | Avg viable | Avg leaky | Average dead | | | |
| (h) | Flask ID | cells/ml | cells/ml | cells/ml | Total | Viability % | Leaky % |
| 40 | Neg | 9.6E+05 | 5.0E+04 | 0.0E+00 | 1.0E+06 | 95 | 5 |
| | control | | | | | | |
| | A | 4.9E+05 | 7.0E+04 | 1.7E+05 | 7.3E+05 | 68 | 10 |
| | B | 3.2E+05 | 1.9E+05 | 1.3E+05 | 6.4E+05 | 50 | 30 |
| | C | 4.3E+06 | 1.1E+06 | 8.4E+05 | 6.3E+06 | 68 | 18 |
| | D | 5.2E+06 | 4.1E+05 | 5.0E+05 | 6.1E+06 | 85 | 7 |
| | E | 5.6E+05 | 2.4E+06 | 7.4E+05 | 3.7E+06 | 15 | 64 |
| | F | 2.7E+06 | 5.8E+05 | 3.4E+05 | 3.6E+06 | 75 | 16 |
| | G | 3.7E+06 | 7.8E+05 | 4.6E+05 | 4.9E+06 | 75 | 16 |
| | H | 4.3E+05 | 2.0E+05 | 1.2E+05 | 7.5E+05 | 58 | 26 |
| | I | 2.4E+06 | 5.3E+05 | 3.0E+05 | 3.2E+06 | 74 | 16 |
| 48 | A | 3.0E+05 | 7.0E+04 | 5.0E+04 | 4.2E+05 | 71 | 17 |
| | B | 1.2E+06 | 2.7E+05 | 1.6E+05 | 1.6E+06 | 74 | 17 |
| | C | 5.2E+06 | 7.4E+05 | 3.1E+05 | 6.2E+06 | 83 | 12 |
| | D | 5.2E+06 | 8.9E+05 | 4.0E+05 | 6.5E+06 | 80 | 14 |
| | E | 2.8E+06 | 9.8E+05 | 3.6E+05 | 4.1E+06 | 68 | 24 |
| | F | 2.3E+06 | 9.1E+05 | 3.0E+05 | 3.5E+06 | 65 | 26 |
| | G | 2.9E+06 | 2.3E+06 | 4.5E+05 | 5.6E+06 | 51 | 41 |
| | H | 7.6E+05 | 6.2E+05 | 1.9E+05 | 1.6E+06 | 49 | 39 |
| | I | 1.7E+06 | 1.1E+06 | 4.3E+05 | 3.3E+06 | 53 | 34 |
| 65 | FA | 4.5E+05 | 7.7E+05 | 1.5E+05 | 1.4E+06 | 33 | 56 |
| | B | 3.4E+05 | 8.5E+05 | 9.5E+04 | 1.3E+06 | 27 | 66 |
| | C | 4.0E+06 | 1.9E+06 | 8.8E+04 | 6.0E+06 | 67 | 31 |
| | D | 4.6E+06 | 1.9E+06 | 1.0E+05 | 6.6E+06 | 69 | 29 |
| | E | 1.9E+06 | 1.6E+06 | 1.5E+05 | 3.6E+06 | 52 | 44 |
| | F | 1.9E+06 | 1.6E+06 | 7.5E+04 | 3.5E+06 | 53 | 44 |
| | G | 2.3E+06 | 3.8E+06 | 2.6E+05 | 6.3E+06 | 36 | 60 |
| | H | 3.8E+05 | 1.0E+06 | 5.5E+04 | 1.5E+06 | 26 | 70 |
| | I | 1.1E+06 | 1.8E+06 | 2.4E+05 | 3.1E+06 | 34 | 58 |
| 72 | A | 3.9E+05 | 1.1E+06 | 1.0E+05 | 1.6E+06 | 25 | 69 |
| | B | 2.8E+05 | 9.3E+05 | 1.1E+05 | 1.3E+06 | 21 | 70 |
| | C | 4.7E+06 | 2.1E+06 | 1.8E+05 | 7.0E+06 | 67 | 30 |
| | D | 4.5E+06 | 2.3E+06 | 1.9E+05 | 7.0E+06 | 64 | 33 |
| | E | 4.9E+05 | 4.0E+05 | 2.5E+04 | 9.1E+05 | 53 | 44 |
| | F | 2.0E+06 | 2.2E+06 | 3.8E+04 | 4.2E+06 | 48 | 51 |
| | G | 1.5E+06 | 3.6E+06 | 2.5E+05 | 5.4E+06 | 28 | 68 |
| | H | 3.2E+05 | 9.3E+05 | 6.5E+04 | 1.3E+06 | 24 | 71 |
| | I | 9.1E+05 | 2.5E+06 | 1.5E+05 | 3.5E+06 | 26 | 70 |
| 96 | A | 8.8E+04 | 9.1E+05 | 5.8E+04 | 1.1E+06 | 8 | 86 |
| | B | 1.2E+05 | 1.1E+06 | 4.0E+04 | 1.3E+06 | 9 | 87 |
| | C | 6.1E+05 | 5.4E+06 | 1.1E+05 | 6.1E+06 | 10 | 88 |
| | D | 6.5E+05 | 5.1E+06 | 1.0E+05 | 5.8E+06 | 11 | 87 |
| | E | 3.8E+05 | 2.6E+06 | 5.0E+04 | 3.0E+06 | 13 | 86 |
| | F | 4.6E+05 | 3.0E+06 | 6.3E+04 | 3.5E+06 | 13 | 85 |
| | G | 3.3E+05 | 4.4E+06 | 3.5E+05 | 5.1E+06 | 6 | 87 |
| | H | 1.5E+05 | 9.5E+05 | 4.0E+04 | 1.1E+06 | 13 | 83 |
| | I | 2.3E+05 | 2.4E+06 | 1.3E+05 | 2.8E+06 | 8 | 87 |

| | | | | | | |
|---|---|---|---|---|---|---|
| Neg control | 3.1E+06 | 1.7E+05 | 0.0E+00 | 3.3E+06 | 95 | 5 |

TABLE 5
AEX-HPLC assay results of ColoAd1 in Supernatant (SN) and CVL (intracellular)

| | | | Produced | Total vp | Produced | Produced | |
|---|---|---|---|---|---|---|---|
| Infection | Sample | Total vp | vp/cell | (% in | vp/cell | vp/cell | Total vp |
| time (h) | Detail | (% in SN) | (SN) | CVL) | (CVL) | (SN + CVL) | yield/flask |
| 40 | Flask A | 0 | 0 | 100 | 41222 | 41222 | 1.65 × 10¹² |
| 48 | Flask A | 20 | 15509 | 80 | 61724 | 77232 | 3.09 × 10¹² |
| 65 | Flask A | 69 | 127823 | 31 | 56114 | 183937 | 7.36 × 10¹² |
| 72 | Flask A | 98 | 163705 | 2 | 3274 | 166980 | 6.68 × 10¹² |
| 96 | Flask A | 93 | 185298 | 7 | 13893 | 199192 | 7.97 × 10¹² |
| 40 | Flask B | 0 | 0 | 100 | 36293 | 36293 | 1.45 × 10¹² |
| 48 | Flask B | 18 | 19867 | 82 | 89433 | 109301 | 4.37 × 10¹² |
| 65 | Flask B | 71 | 126002 | 29 | 52553 | 178555 | 7.14 × 10¹² |
| 72 | Flask B | 98 | 147558 | 2 | 2951 | 150510 | 6.02 × 10¹² |
| 96 | Flask B | 93 | 184024 | 7 | 13070 | 197094 | 7.88 × 10¹² |
| 40 | Flask C | 0 | 0 | 100 | 5843 | 5843 | 9.35 × 10¹¹ |
| 48 | Flask C | 0 | 0 | 100 | 5706 | 5706 | 9.13 × 10¹¹ |
| 65 | Flask C | 0 | 0 | 100 | 3767 | 3767 | 6.03 × 10¹¹ |
| 72 | Flask C | 0 | 0 | 100 | 3631 | 3631 | 5.81 × 10¹¹ |
| 96 | Flask C | 0 | 0 | 0 | 0 | 0 | 0 |
| 40 | Flask D | 0 | 0 | 100 | 6001 | 6001 | 9.60 × 10¹¹ |
| 48 | Flask D | 0 | 0 | 100 | 6772 | 6772 | 1.08 × 10¹² |
| 65 | Flask D | 0 | 0 | 100 | 3488 | 3488 | 5.58 × 10¹¹ |
| 72 | Flask D | 0 | 0 | 100 | 4178 | 4178 | 6.68 × 10¹¹ |
| 96 | Flask D | 0 | 0 | 0 | 0 | 0 | 0 |
| 40 | Flask E | 0 | 0 | 100 | 20149 | 20149 | 2.01 × 10¹² |
| 48 | Flask E | 30 | 7142 | 70 | 16276 | 23418 | 2.34 × 10¹² |
| 65 | Flask E | 56 | 11305 | 44 | 8813 | 20117 | 2.01 × 10¹² |
| 72 | Flask E | 64 | 14559 | 36 | 8302 | 22861 | 2.29 × 10¹² |
| 96 | Flask E | 85 | 17386 | 15 | 3172 | 20558 | 2.06 × 10¹² |
| 40 | Flask F | 0 | 0 | 100 | 25088 | 25088 | 2.51 × 10¹² |
| 48 | Flask F | 26 | 7574 | 74 | 21086 | 28660 | 2.87 × 10¹² |
| 65 | Flask F | 57 | 12324 | 43 | 9413 | 21737 | 2.17 × 10¹² |
| 72 | Flask F | 69 | 18240 | 31 | 8220 | 26460 | 2.65 × 10¹² |
| 96 | Flask F | 88 | 20295 | 12 | 2725 | 23020 | 2.30 × 10¹² |
| 40 | Flask G | 11 | 7003 | 89 | 57008 | 64010 | 6.40 × 10¹² |
| 48 | Flask G | 38 | 30548 | 62 | 48833 | 79382 | 7.94 × 10¹² |
| 65 | Flask G | 59 | 46073 | 41 | 31699 | 77772 | 7.78 × 10¹² |
| 72 | Flask G | 66 | 57663 | 34 | 29199 | 86862 | 8.69 × 10¹² |
| 96 | Flask G | 81 | 65738 | 19 | 15773 | 81510 | 8.15 × 10¹² |
| 40 | Flask H | 28 | 19075 | 72 | 49192 | 68267 | 2.73 × 10¹² |
| 48 | Flask H | 53 | 55309 | 47 | 48091 | 103400 | 4.14 × 10¹² |
| 65 | Flask H | 79 | 98126 | 21 | 25714 | 123841 | 4.95 × 10¹² |
| 72 | Flask H | 81 | 107729 | 19 | 25036 | 132765 | 5.31 × 10¹² |
| 96 | Flask H | 92 | 105893 | 8 | 8775 | 114668 | 4.59 × 10¹² |
| 40 | Flask I | 19 | 6169 | 81 | 26235 | 32404 | 5.18 × 10¹² |
| 48 | Flask I | 39 | 20457 | 61 | 31430 | 51887 | 8.30 × 10¹² |
| 65 | Flask I | 71 | 38093 | 29 | 15738 | 53831 | 8.61 × 10¹² |
| 72 | Flask I | 72 | 44409 | 28 | 17336 | 61746 | 9.88 × 10¹² |
| 96 | Flask I | 81 | 47634 | 19 | 11180 | 58814 | 9.41 × 10¹² |

### Example 12

Key DOE parameters indicated from the experiment described in Example 11 in shake flasks were assessed in a 5 L bioreactor. HEK293 cell expansion and bioreactor preparation was performed as described in Example 9. Cell counting was performed using automatic cell counter and trypan blue staining. Once viable cells totaling 7.5e⁸ cells were attained in shake flasks, the cells were used to inoculate the 5 L bioreactor. The bioreactor was infected with EnAd at an MOI of 12.5 ppc when the target cell density of 1e⁶ cell/mL was achieved.

At 24, 40, 48, 65 and 70 hrs post infection samples (20 ml) were taken for cell count, viability and total virus concentration. The supernatant was separated from the cells by centrifugation and stored at −80° C. until viral particle concentrations analysis by AEX-HPLC was performed. The cell pellet samples were prepared as outlined in Example 11 and stored at −80° C. until HPLC analysis. The results are shown in Tables 6.

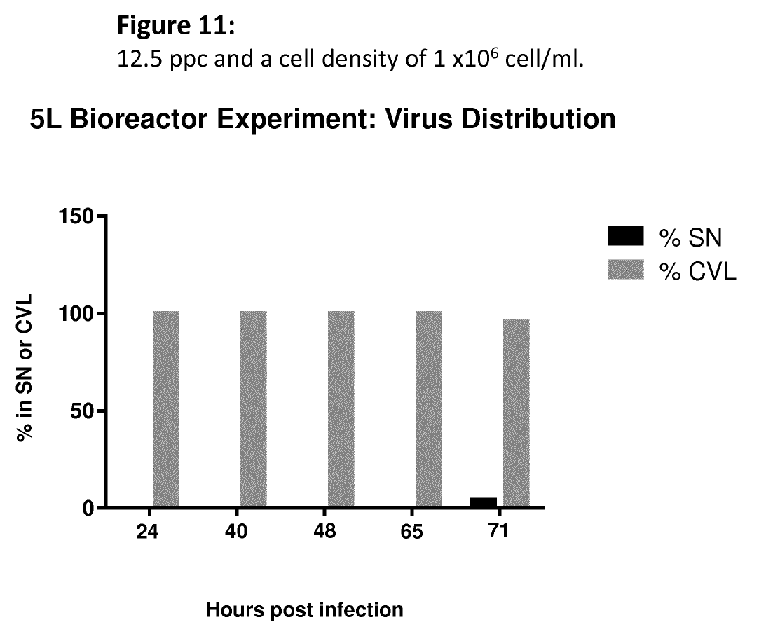

Under these conditions, the majority of the virus was present in the CVL for all time points (FIG. 11) with no significant virus present in the supernatant until 71 h post infection. At 71 hrs post-infection, 96% of EnAd virus was observed in the cell pellet (Table 6 and FIG. 11) with only 4% present in supernatant (Table 6). A total of 428,868 virus particles were extracted from the cells by chemical lysis and cell debris removed by clarification (refer to post lysis numbers in table 6).

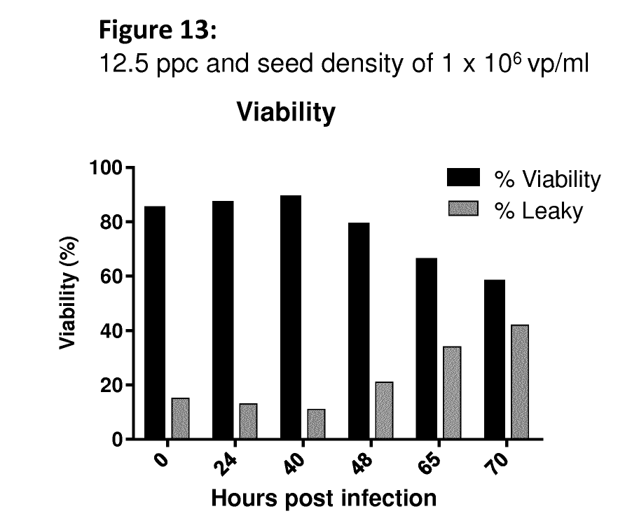

Cell viability at 71 hour post infection with 12.5 ppc was 58% compared to 85% at TO (FIG. 13). At 71 hour post infection with 50 ppc (see Example 13), the cell viability was typically below 30%.

TABLE 6
AEX-HPLC assay results of EnAd

| Infec- | | | | | | | |
|---|---|---|---|---|---|---|---|
| tion | | | | | | | Total vp |
| time | total vp | % | total vp | % | vp/cell | vp/cell | yield from |
| point | SN | SN | CVL | CVL | (SN) | (CVL) | bioreactor |
| 24 | 0.00E+00 | 0 | 2.37E+13 | 100 | 0 | 7914 | 2.40E+13 |
| 40 | 0.00E+00 | 0 | 4.74E+13 | 100 | 0 | 15796 | 4.70E+13 |
| 48 | 0.00E+00 | 0 | 6.18E+13 | 100 | 0 | 20605 | 6.20E+13 |
| 65 | 0.00E+00 | 0 | 3.79E+14 | 100 | 0 | 126367 | 3.80E+14 |
| 71 | 2.81E+13 | 4 | 6.06E+14 | 96 | 9383 | 202069 | 6.30E+14 |
| Post | 1.28E+15 | 99 | 8.67E+12 | 1 | 425979 | 2889 | 1.30E+15 |
| lysis | | | | | | | |

### Example 13

An experimental protocol as described in Example 12 was employed with a target cell density at infection of 1.9e⁶ cells/ml and the culture was infected with EnAd at an MOI of 50 ppc. Samples were taken at 24, 48, 60 and 70 hrs post-infection. The viral particle concentrations of the samples were analysed with AEX-HPLC and the results are shown in Tables 7.

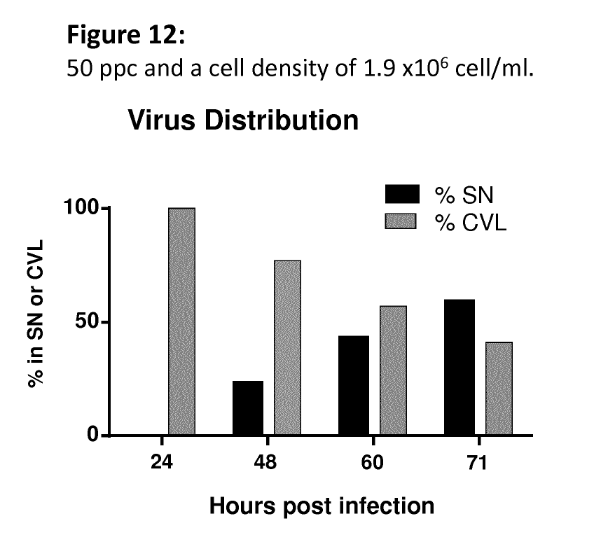

At 71 hrs post-infection, 59% (89, 485 vp) of EnAd virus was observed in the supernatant (Table 8) with 41% (63, 081 vp) present in the cell viral lysate (Table 7 and FIG. 12). A total of 213, 981 virus particles were extracted from the cells by chemical lysis and cell debris removed by clarification (Refer to post lysis numbers in Table 7).

In this experiment, the bioreactor culture parameters of 1.9e⁶ cell/mL infected with 50 ppc produced half the yield (213981 vp/cell) compared to the parameters outlined in Example 12 where 428,868 vp/cell were produced at 1e6 cell/mL infected with 12.5 ppc.

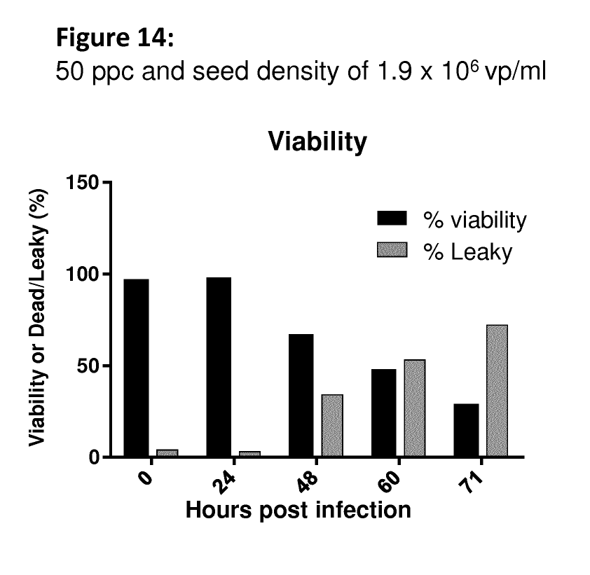

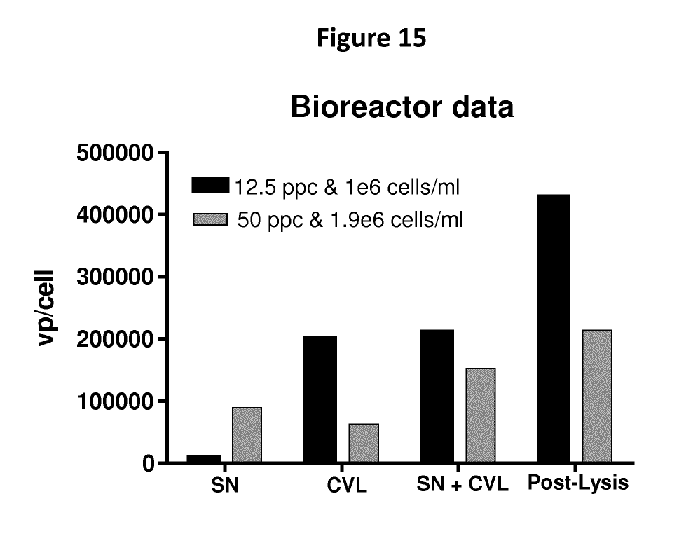

Cell viability at 71 hour post infection with 50 ppc was 26% compared to 96% at TO (FIG. 14). Cell viability at 71 hour post infection with 12.5 ppc (in Example 12) was 58% compared to 85% at TO (FIG. 13). The low MOI (12.5 ppc) at infection may account for the higher (post infection) cell viability (58%) at 71 h which potentially contributed to the 2 fold higher viral production post lysis in Example 12 compared to this study (FIG. 15).

TABLE 7
AEX-HPLC assay results of EnAd

| Infection | | | | | | | Total vp |
|---|---|---|---|---|---|---|---|
| time | total vp | | total vp | | vp/cell | vp/cell | yield form |
| point | SN | % SN | CVL | % CVL | (SN) | (CVL) | bioreactor |
| 24 | 0.00E+00 | 0 | 1.57E+14 | 100 | 0 | 27550 | 1.60E+14 |
| 48 | 1.95E+14 | 23 | 6.49E+14 | 77 | 34141 | 113941 | 8.40E+14 |
| 60 | 3.59E+14 | 43 | 4.80E+14 | 57 | 62966 | 84245 | 8.40E+14 |
| 71 | 5.10E+14 | 59 | 3.60E+14 | 41 | 89485 | 63081 | 8.70E+14 |
| Post lysis | 1.11E+15 | 100 | 0.00E+00 | 0 | 213981 | 0 | 1.20E+15 |

### Example 14

An experimental protocol was employed as described in Example 1 but with a shaker flask working volume of 25 ml, cell density of 2.2×10⁶ viable cells/mL and 50 ppc infection. To explore principles of a continuous manufacturing approach, at various time points 20 mL of the 25 mL cell suspension (80%) was removed and replaced with 20 mL non-infected cells at the same cell density as at the start (2.2×10⁶ viable cells/mL) in fresh medium. The experiment was continued for 7 days and on each day (Day 1, Day 2, Day 3, Day 4, Day 5, Day 6 and Day 7) the 20 mL post infection samples were taken for cell count, viability and total virus concentrations in supernatant and CVL. Post infection cell counting was performed using Hemocytometer and Trypan Blue stain (Invitrogen). The supernatant was separated from the cells by centrifugation and stored at −80° C. for viral particle concentration analysis by AEX-HPLC. The cell pellet samples were prepared as outlined in Example 11 and stored at −80° C. until HPLC analysis. The viability results are shown in Table 8 and the HPLC results for supernatant and CVL are shown in Table 9.

Control shaker flasks as the experimental control for this experiment were set up with a working volume of 25 ml, cell density of 2.2×10⁶ viable cells/mL and infected with 50 ppc. No cell suspension removal or addition of fresh cells and medium were undertaken with these control flasks post infection. These controls were terminated at Day 3 post infection. Cell count and viability were assessed daily and day 3 post-infection samples were taken for cell count, viability and total virus concentrations. Samples were processed as described in Example 11 for supernatant and CVL analysis by HPLC. The results are shown in Table 9.

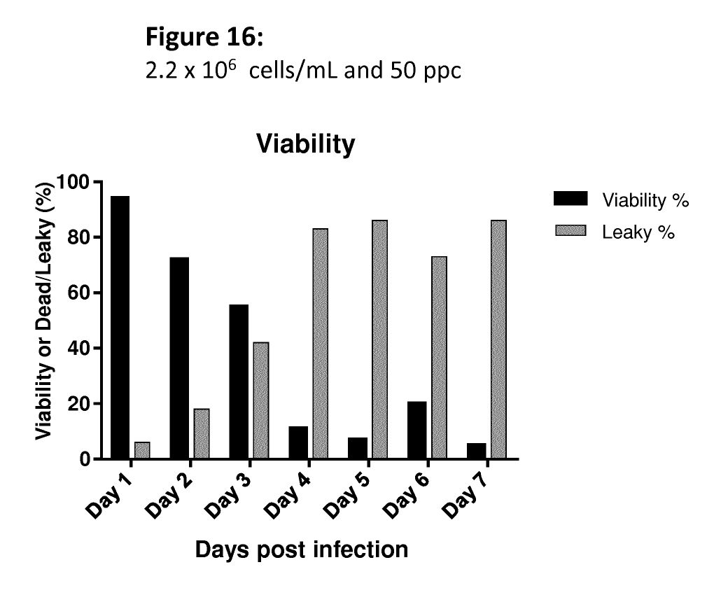

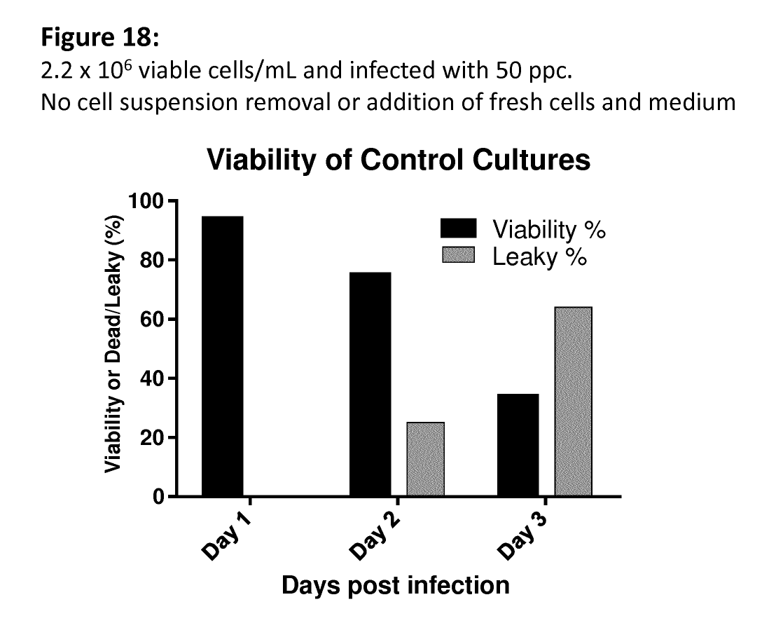

The cell viability on Day 1 was 94% which decreased overtime with the lowest viability at 5% on Day 7. The percentage of leaky cells increased over time with the highest amount present on Day 7(86%) (Refer to Table 8 and FIG. 16). The control cell viability at Day 1 was also 94% which decreased with the lowest viability on Day 3 (34%). The percentage leaky cells increased over time with the highest on Day 3 (64%) (Table 8 and FIG. 18).

TABLE 8
Viability data in shaker flasks

| | Avg | Avg | Avg | | | | | | |
|---|---|---|---|---|---|---|---|---|---|
| | viable | leaky | dead | Avg | Avg | Avg | | | |
| Timepoint | cells 8 | cells 8 | cells 8 | viable | (leaky) | dead | | | |
| (h) | squares | squares | squares | cells | cells | cells | Total | Viability % | Leaky % |
| Day 1 | 55 | 0 | 3.5 | 2.20E+06 | 0.00E+00 | 0.00E+00 | 2.00E+06 | 94 | 6 |
| Day 2 | 47.75 | 10.5 | 0 | 1.91E+06 | 4.20E+05 | 0.00E+00 | 2.33E+06 | 72 | 18 |
| Day 3 | 32.5 | 22.5 | 2.25 | 6.50E+05 | 4.95E+05 | 4.50E+04 | 1.19E+06 | 55 | 42 |
| Day 4 | 12 | 85.5 | 7.25 | 2.40E+05 | 1.86E+06 | 1.45E+05 | 2.24E+06 | 11 | 83 |
| Day 5 | 7 | 75.25 | 6.5 | 1.40E+05 | 1.64E+06 | 1.30E+05 | 1.91E+06 | 7 | 86 |
| Day 6 | 21 | 69.75 | 8 | 4.20E+05 | 1.56E+06 | 1.60E+05 | 2.14E+06 | 20 | 73 |
| Day 7 | 5.75 | 83.25 | 8.75 | 1.15E+05 | 1.84E+06 | 1.75E+05 | 2.13E+06 | 5 | 86 |
| Control | 55 | 0 | 3.5 | 2.20E+06 | 0.00E+00 | 0.00E+00 | 2.00E+06 | 94 | 6 |
| Day 1 | | | | | | | | | |
| Control | 20 | 6.5 | 0 | 8.00E+05 | 2.60E+05 | 0.00E+00 | 1.06E+06 | 75 | 25 |
| Day 2 | | | | | | | | | |
| Control | 64.75 | 120 | 4 | 1.30E+06 | 2.40E+06 | 8.00E+04 | 3.78E+06 | 34 | 64 |
| Day 3 | | | | | | | | | |

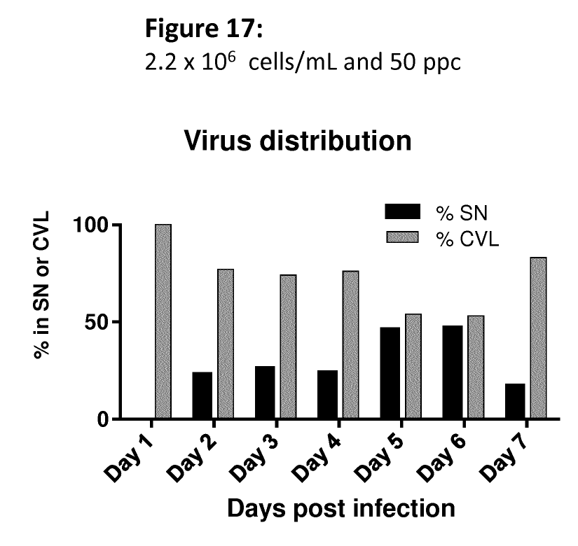

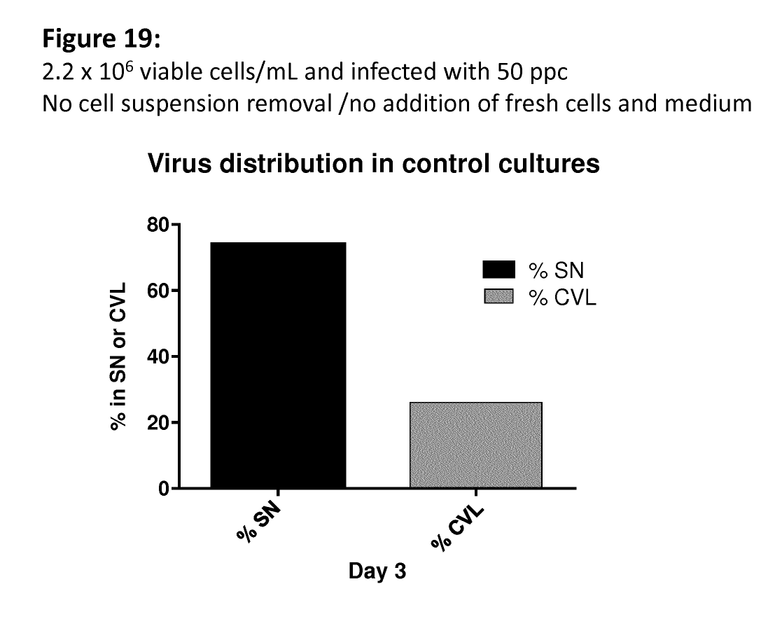

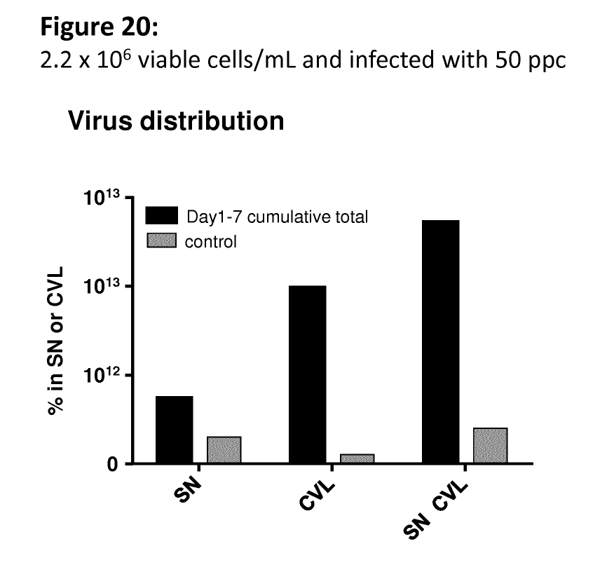

The percentage of virus in the supernatant varied from day 3 to day 7 with maximum on day 5 and 6 (46 & 47%, respectively). By day 7 only 17% was present in the supernatant (Table 9 and FIG. 17). The control at day 3 had 74% of the virus present in the supernatant (FIG. 19). For the control CVL, at day 3 only 26% virus remained in the cell, where as in the continuous manufacturing test flasks 53% remained in the cell from day 1 to 7 with 83% present in the cell at day 7 (Table 9, FIG. 19) For this study, the total cumulative viral particles generated by the daily addition of fresh non-infected cell to the infected cell cultures over 7 days was 1.36e¹³ vp which was 7-fold higher than the amount generated in the control flasks (2.0e¹² vp). The total amount in the supernatant over 7 days was 3.7e¹² vp which represented 27% of the total vp (1.36e¹³ vp), with 73% present in the CVL during the 7 days. In comparison, the viral distribution of the control was 74% (1.5e¹² vp) present in the supernatant with 26% (5.2e¹¹ vp) present in the CVL (Table 9 and 10, FIG. 20).

TABLE 9
AEX-HPLC assay results of EnAd

| Days | | | | | | | | |
|---|---|---|---|---|---|---|---|---|
| post | Total vp | | Total vp | | vp/cell | vp/cell | vp/cell | |
| infection | SN | % SN | CVL | % CVL | (SN) | (CVL) | (SN + CVL) | Total vp |
| Day1 | 0.0E+00 | 0% | 4.4E+11 | 100% | 0 | 10104 | 10104 | 4.45E+11 |
| Day2 | 3.3E+11 | 23% | 1.1E+12 | 77% | 7560 | 24973 | 32533 | 1.43E+12 |
| Day3 | 6.8E+11 | 26% | 1.9E+12 | 74% | 15367 | 43736 | 59103 | 2.60E+12 |
| Day4 | 7.5E+11 | 24% | 2.4E+12 | 76% | 17055 | 54515 | 71570 | 3.15E+12 |
| Day5 | 9.6E+11 | 46% | 1.1E+12 | 54% | 21859 | 25821 | 47680 | 2.10E+12 |
| Day6 | 5.3E+11 | 47% | 6.0E+11 | 53% | 12002 | 13677 | 25679 | 1.13E+12 |
| Day7 | 4.6E+11 | 17% | 2.3E+12 | 83% | 10497 | 52621 | 63118 | 2.78E+12 |
| Control | 1.5E+12 | 74% | 5.2E+11 | 26% | 27435 | 9522 | 36957 | 2.0E+12 |

TABLE 10
Total virus particles

| Experiment | Total VP SN | Total VP CVL | Total VP |
|---|---|---|---|
| Cumulative Total | 3.71E+12 (27%) | 9.84E+12 (73%) | 1.36E+13 |
| VP (Day 1-7) | | | |
| Control | 1.5E+12 (74%) | 5.2E+11 (26%) | 2.0E+12 |

### Example 15

An experimental protocol was employed as described in Example 14 but with a shaker flask working volume of 50 ml, cell density of 1.0×10⁶ viable cells/mL and 12.5 ppc infection. At various time points 40 mL of the 50 mL suspension (80%) was removed and replaced with 40 mL uninfected cells at 1.0×10⁶ viable cells/mL in fresh medium. The experiment was continued for 5 days and on each day (Day 1, Day 2, Day 3, Day 4 and Day 5) post infection samples were taken for cell count, viability and total virus concentration. Post infection cell counting was performed using Hemocytometer and Trypan Blue stain (Invitrogen). The supernatant was separated from the cells by centrifugation and stored at −80° C. until viral particle concentrations analysis by AEX-HPLC was performed. The cell pellet samples was prepared as outlined in Example 11 and stored at −80° C. until HPLC analysis. The viability results are shown in Tables 11. The HPLC results for supernatant and CVL are in Table 12.

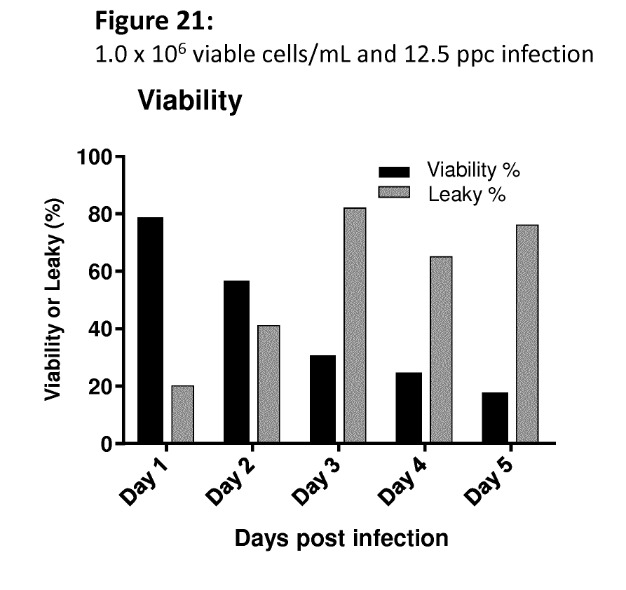

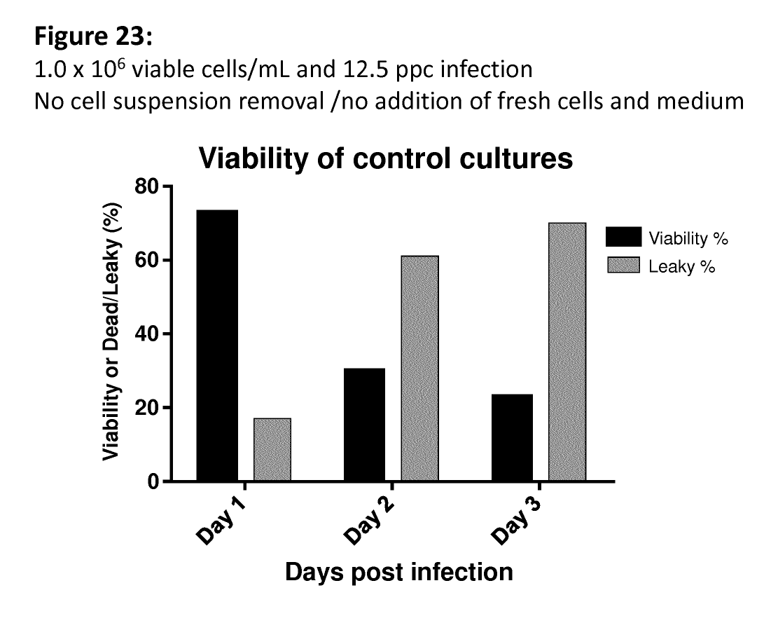

The cell viability on Day 1 was 78% which decreased overtime with the lowest viability at Day 7(16%). The percentage leaky cells increased over time with highest amount present on Day 3 (82%) (Table 11 and FIG. 21). The control cell viability at Day 1 was 73% which decreased with the lowest viability recorded on Day 3(23%). The percentage leaky cells increased over time and highest on Day 3(70%) (FIG. 23).

TABLE 11
Viability data of shaker flasks

| | Avg | Avg | Avg | | | | | | |
|---|---|---|---|---|---|---|---|---|---|
| | viable | leaky | dead | Avg | | | | | |
| Timepoint | cells 8 | cells 8 | cells 8 | viable | Avg leaky | Avg dead | | | |
| (h) | squares | squares | squares | cells | cells | cells | Total | Viability % | Leaky % |
| Day 1 | 50.3 | 12.8 | 1.3 | 1.01E+06 | 2.55E+05 | 2.50E+04 | 1.29E+06 | 10.5 | 20 |
| Day 2 | 53.3 | 22.5 | 2.3 | 1.07E+06 | 4.50E+05 | 4.50E+04 | 1.56E+06 | 56 | 41 |
| Day 3 | 3.8 | 27.5 | 6.3 | 7.50E+04 | 5.50E+05 | 1.25E+05 | 7.50E+05 | 30 | 82 |
| Day 4 | 6.8 | 28.5 | 6.0 | 1.35E+05 | 5.70E+05 | 1.20E+05 | 8.25E+05 | 24 | 65 |
| Day 5 | 8.0 | 34.3 | 2.3 | 1.60E+05 | 6.85E+05 | 4.50E+04 | 8.90E+05 | 17 | 76 |
| Control | 18 | 4 | 3 | 7.35E+05 | 1.68E+05 | 1.03E+05 | 1.01E+06 | 73 | 17 |
| Day-1 | | | | | | | | | |
| Control | 20 | 40 | 6 | 3.93E+05 | 8.06E+05 | 1.20E+05 | 1.32E+06 | 30 | 61 |
| Day-2 | | | | | | | | | |
| Control | 17 | 50 | 5 | 3.34E+05 | 1.00E+06 | 1.06E+05 | 1.44E+06 | 23 | 70 |
| Day-3 | | | | | | | | | |

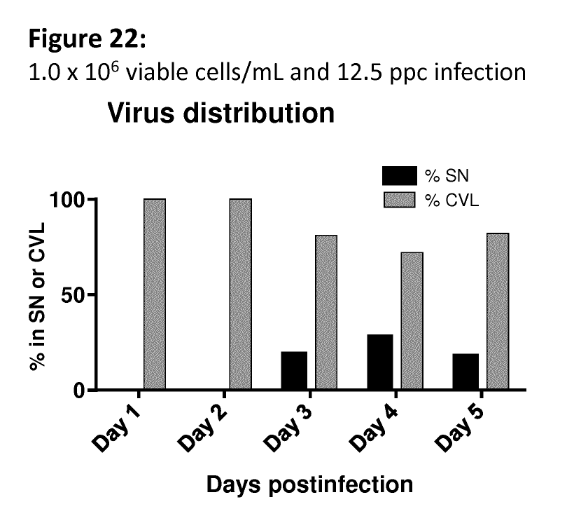

The percentage of virus in the supernatant varied from day 1 to day 5 with maximum on day 4 (28%, respectively). By day 5 only 18% was present in the supernatant (Table 12 and FIG. 22). The control (flasks which did not have removal and replacement of cells and medium) at day 3 had 81% of the virus present in the supernatant and 19% in CVL.

For this control CVL, at day 3 only 19% virus remained in the cell, where as in the flasks having daily cell and media replacements ≧72% remained cell associated from day 1 to 5 with 82% present in the cell pellets at day 5 (Table 12).

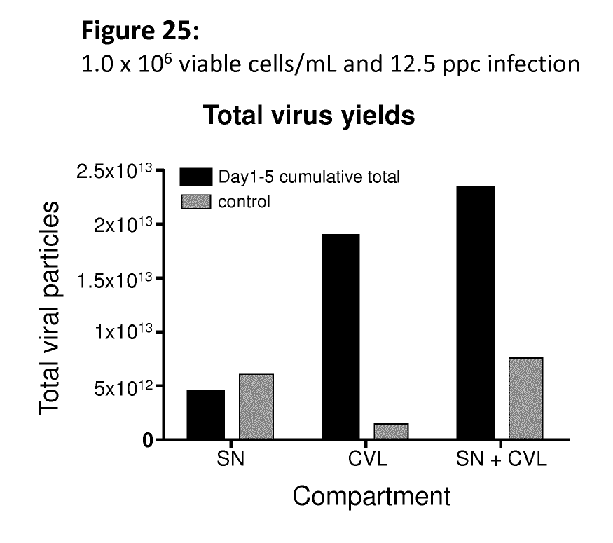

In this study, the total viral particles generated by the addition of fresh non-infected cells to infected cells over 5 days was 2.33e¹³ vp which was 3-fold higher than the amount generated in the control (7.6e¹²). The total amount in the supernatant over 5 days was 4.4e¹² vp which represented 19% of the total vp (2.33e¹³), with 81% present in the CVL during the 5 days. In comparison, the viral distribution of the control cultures was 81% (6.10e¹²) present in the supernatant with 19% (1.48e¹²) present in the CVL (Table 12 and 13, FIG. 25).

TABLE 12
AEX-HPLC assay results of EnAd

| | Total VP | | Total VP | | vp/cell | vp/cell | vp/cell | |
|---|---|---|---|---|---|---|---|---|
| Sample ID | SN | % SN | CVL | % CVL | (SN) | (CVL) | (SN + CVL) | Total VP |
| Day 1 | 0.0E+00 | 0% | 6.1E+11 | 100% | 0 | 15357 | 15357 | 6.1E+11 |
| Day 2 | 0.0E+00 | 0% | 2.6E+12 | 100% | 0 | 63820 | 63820 | 2.6E+12 |
| Day 3 | 1.2E+12 | 19% | 5.1E+12 | 81% | 29834 | 128393 | 158227 | 6.3E+12 |
| Day 4 | 2.0E+12 | 28% | 5.2E+12 | 72% | 50298 | 129956 | 180254 | 7.2E+12 |
| Day 5 | 1.2E+12 | 18% | 5.4E+12 | 82% | 30008 | 134004 | 164012 | 6.6E+12 |
| control | 6.1E+12 | 81% | 1.48E+12 | 19% | 153565 | 36964 | 190529 | 7.6E+12 |

TABLE 13
Total virus particles

| Experiment | Total VP SN | Total VP CVL | Total VP |
|---|---|---|---|
| Cumulative Total | 4.40E+12 | 1.89E+13 | 2.33E+13 |
| VP (Day 1-5) | | | |
| control | 6.10E+12 | 1.48E+12 | 7.60E+12 |

### Example 16

An experimental protocol was employed as described in Example 15 but at various time points 47.5 mL of the 50 mL suspension (95%) was removed and replaced with 47.5 mL uninfected cells in fresh medium at the same cell density of 1.0×10⁶ viable cells/mL. This study was run in parallel with that in Example 15 and used the same control flasks. The viability results are shown in Tables 14. The HPLC results for supernatant and CVL are shown in Table 15.

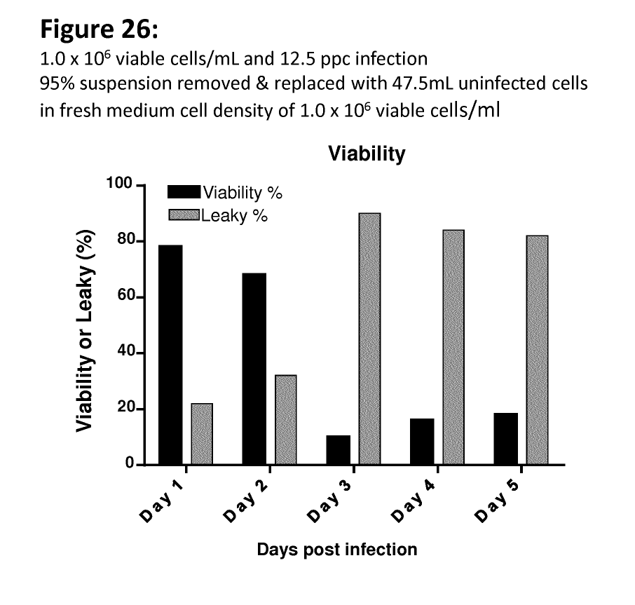

The cell viability on Day 1 was 78% which decreased overtime with the lowest viability at Day 5 (16%). The percentage leaky cells increased over time with highest amount present on Day 5 (77%) (Table 14 and FIG. 26). The control cell viability at Day 1 was 73% which decreased with the lowest viability recorded on Day 3 (23%). The percentage leaky cells increased over time and highest on Day 3 (70%) (FIG. 23).

TABLE 14
Viability data of shaker flasks

| | Avg viable | Avg leaky | Avg dead | | | |
|---|---|---|---|---|---|---|
| Timepoint (h) | cells | cells | cells | Total | Viability % | Leaky % |
| Day 1 | 1.01E+06 | 2.55E+05 | 2.50E+04 | 1.29E+06 | 78 | 20 |
| Day 2 | 1.07E+06 | 4.50E+05 | 4.50E+04 | 1.56E+06 | 68 | 29 |
| Day 3 | 7.50E+04 | 5.50E+05 | 1.25E+05 | 7.50E+05 | 30 | 73 |
| Day 4 | 1.35E+05 | 5.70E+05 | 1.20E+05 | 8.25E+05 | 16 | 69 |
| Day 5 | 1.60E+05 | 6.85E+05 | 4.50E+04 | 8.90E+05 | 18 | 77 |
| Control Day-1 | 7.35E+05 | 1.68E+05 | 1.03E+05 | 1.01E+06 | 73 | 17 |
| Control Day-2 | 3.93E+05 | 8.06E+05 | 1.20E+05 | 1.32E+06 | 30 | 61 |
| Control Day-3 | 3.34E+05 | 1.00E+06 | 1.06E+05 | 1.44E+06 | 23 | 70 |

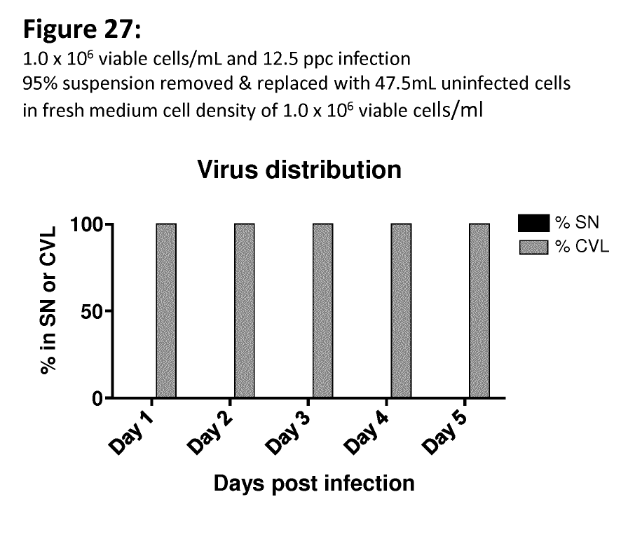

From Day 1 to 5 no virus present in the supernatant, all viruses remained in CVL (Table 15 and FIG. 27).

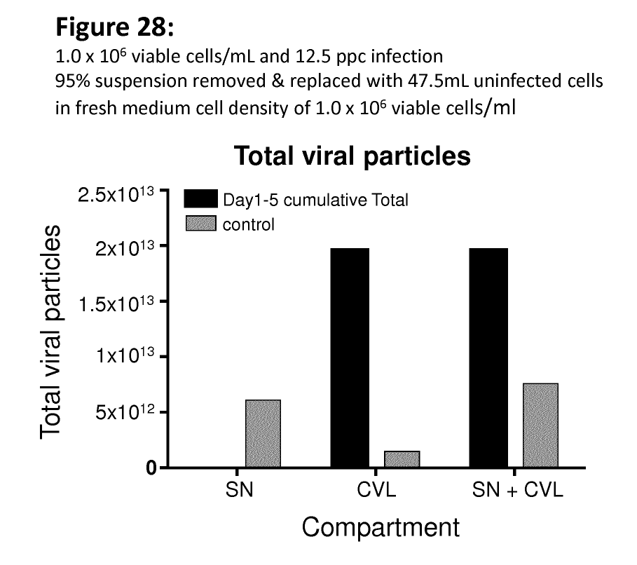

For this experiment, the total viral particles generated by the daily removal of suspension and addition of fresh non-infected cell to infected cells over 5 days was 1.96e¹³ vp which was 3 fold higher than the amount generated in the control (7.6e¹²). The total amount of virus in the CVL over 5 days was 1.96e¹³ vp which represented 100% of the total vp (1.96e¹³). In comparison, the viral distribution of the control was 81% (6.10e¹²) present in the supernatant with 19% (1.48e¹²) present in the CVL (Table 16 and FIG. 28).

TABLE 15
AEX-HPLC assay results of ColoAd1

| | Total VP | | Total VP | | vp/cell | vp/cell | vp/cell | |
|---|---|---|---|---|---|---|---|---|
| Sample ID | SN | % SN | CVL | % CVL | (SN) | (CVL) | (SN + CVL) | Total VP |
| Day 1 | 0.00E+00 | 0% | 9.7E+11 | 100% | 0 | 20513 | 20513 | 9.7E+11 |
| Day 2 | 0.00E+00 | 0% | 1.5E+12 | 100% | 0 | 31209 | 31209 | 1.5E+12 |
| Day 3 | 0.00E+00 | 0% | 5.0E+12 | 100% | 0 | 105575 | 105575 | 5.0E+12 |
| Day 4 | 0.00E+00 | 0% | 5.8E+12 | 100% | 0 | 122263 | 122263 | 5.8E+12 |
| Day 5 | 0.00E+00 | 0% | 6.3E+12 | 100% | 0 | 133257 | 133257 | 6.3E+12 |
| control | 6.1E+12 | 81% | 1.48E+12 | 19% | 153565 | 36964 | 190529 | 7.6E+12 |

TABLE 16
Total virus particles

| Experiment | Total VP SN | Total VP CVL | Total VP |
|---|---|---|---|
| Cumulative Total | 0.00E+00 | 1.96E+13 | 1.96E+13 |
| VP (Day 1-5) | | | |
| control | 6.10E+12 | 1.48E+12 | 7.60E+12 |

1. A continuous process for the manufacture of an adenovirus wherein the process comprises the steps:
A) continuously culturing, in a vessel, mammalian cells infected with the adenovirus in the presence of media suitable for supporting the cells such that the virus replicates, wherein the cells are capable of supporting viral replication, and
B) isolating from the media the virus produced from step A) wherein the isolation of virus is not subsequent to a cell lysis step,
wherein viable cells for virus infection and production are maintained in the vessel at a level suitable for replicating the virus for the period of continuous manufacture,
wherein the process comprises at least one media change or addition and at at least one time point post infection at least some of the cells are changed or cells are added.

2. A process according to claim 1, wherein the virus has a hexon and fibre from a group B adenovirus.

3. A process according to claim 1, wherein the virus is replication competent.

4. A process according to claim 1, wherein the continuous manufacturing period comprises at least two virus replication cycles.

5. A process according to claim 1, wherein each virus replication cycle is in the range of from 30 to 300 hours.

6. A process according to claim 1, wherein the process produces at least 50,000 virus particles per cell at one or more time points post infection.

7. A process according to claim 1, wherein viable cells for virus infection and production are maintained in the vessel at a level suitable for replicating the virus by the addition of cells to the culture.

8. A process according to claim 1, wherein cells are removed from the cultures at one or more time points post infection.

9. A process according to claim 1, wherein the mammalian cells are selected from the group comprising HEK, CHO, Hela, Vero, A549, PerC6 and GMK, in particular HEK293.

10. A process according to claim 1, wherein the multiplicity of infection is 5 to 50 vp/cell.

11. A process according to claim 1, wherein the cells are infected with a starting concentration of virus of 1-4×10⁶ vp/ml.

12. A process according to claim 1, wherein a perfusion culture is employed.

13. A process according to claim 1, wherein a suspension culture is employed.

14. A process according to claim 1, wherein an adhesion culture is employed.

15. A process according to claim 1, wherein the process further comprises a purification step, selected from a CsCl gradient, chromatography step such as ion-exchange chromatography in particular anion-exchange chromatography, and a combination thereof.

16. A process according to claim 1 which comprises at least one media change or addition.

17. A process according to claim 1, which further comprises formulating the virus in a buffer suitable for storage.

18. A process for the manufacture of an adenovirus comprises the steps:
a. culturing, in a vessel, mammalian cells infected with the adenovirus in the presence of media suitable for supporting the cells such that the virus replicates, wherein the cells are capable of supporting viral replication, wherein the starting seed density of the virus is in the range 1 to 2×10⁶ vp/ml (such as 1×10⁶ vp/ml) and the multiplicity of infection is in the range 5 to 20; and
b. performing a lysis step in the period 24 to 75 hours post virus infection to harvest the virus from the cells.

19. A process according to claim 18, wherein the virus is a group B virus.

20. A process according to claim 18, wherein the process comprises at least one media change or addition.

21. A process according to claim 18, wherein at at least one time point post infection at least some of the cells are changed or cells are added.

22. A virus or formulation obtained or obtainable from this process described in claim 1.
